(19) United States
(12) Patent Application Publication
(10) Pub. No.: US 2024/0075123 A1
(43) Pub. Date: Mar. 7, 2024

(54) TREATMENT OF CANCERS AND INFECTIOUS DISEASES WITH KILLED INTACT BACTERIA

(71) Applicant: Indaptus Therapeutics, Inc., New York, NY (US)

(72) Inventors: Michael James Newman, Del Mar, CA (US); Boyan Veselinov Litchev, San Diego, CA (US)

(21) Appl. No.: 18/497,286

(22) Filed: Oct. 30, 2023

Related U.S. Application Data
(60) Provisional application No. 63/426,245, filed on Nov. 17, 2022.

(51) Int. Cl. A61K 39/108; A61K 9/19; A61K 31/405; A61K 31/675; A61K 38/20; A61K 39/00; A61K 39/39; A61K 39/395; A61K 47/26; A61P 35/00; A61P 35/04; C12N 1/20; C12N 1/36

(52) U.S. Cl. CPC A61K 39/0258; A61K 9/19; A61K 31/405; A61K 31/675; A61K 38/2013; A61K 39/0011; A61K 39/39; A61K 39/3955; A61K 47/26; A61P 35/00; A61P 35/04; C12N 1/20; C12N 1/36; A61K 2039/505; A61K 2039/52; A61K 2039/521; A61K 2039/55594; A61K 2039/585

(57) ABSTRACT

The present disclosure relates generally to compositions, dosage forms, and methods for preventing and treating cancers and infections. An example method entails administering to the patient an effective amount of a composition comprising 1×10⁷ to 500×10⁷ intact, stabilized and substantially non-viable E. coli cells which have been treated in such a way as to result in about 70% to 99% reduction of lipopolysaccharide (LPS)-associated endotoxin activity when measured by the Limulus Amebocyte Lysate (LAL) assay as compared to untreated, wild-type E. coli cells, and wherein the composition contains 124 to 62000 endotoxin units (EU) of LPS.

### CROSS REFERENCE TO RELATED APPLICATIONS

This application claims the benefit under 35 U.S.C. § 119(e) of the U.S. Provisional Application Ser. No. 63/426,245, filed Nov. 17, 2022, the content of which is hereby incorporated by reference in its entirety.

### BACKGROUND

Cancer is the second leading cause of death in the United States. In the past 10 years, the advancement of immunotherapies in cancer has transformed cancer therapy and led to many new treatments. The inhibition of immune checkpoints with blocking antibodies against programmed cell death 1 (PD1) or programmed cell death ligand 1 (PD-L1) and cytotoxic T-lymphocyte antigen 4 (CTLA-4) has produced long-term disease-free survival in several advanced malignancies. However, many patients do not respond to immunotherapies and many who benefit from current immunotherapy eventually progress.

Tumors evade immune surveillance by dampening innate and adaptive immune effectors, restricting neoantigen presentation, and impairing infiltrating immune effector cells. Failure of PD-1/PD-L1 inhibitors may be due to insufficient generation of anti-tumor T cells, exclusion of T cells from tumors, inadequate function of tumor-specific T cells, and/or impaired formation of T cell memory.

Systemic innate and adaptive immunity is required for efficient and durable anti-tumor immune responses. Many of the steps involved in innate and adaptive anti-tumor immune responses, including immune cell production, mobilization, migration, activation, and antigen presentation, take place outside of the tumor environment. These steps are controlled, to a significant extent, by pattern recognition receptors (PRRs), which recognize a wide variety of intrinsic and extrinsic danger, pathogen, and xenobiotic-associated molecular patterns. Toll-like receptors (TLRs) represent the most prominent PRR family, comprising 9 functional TLRs in mammals, with a subset expressed on essentially all immune cells, including monocytes, macrophages, neutrophils, natural killer (NK) cells, γδT-cells, NKT-cells, dendritic cells, CD4+ T cells and CD8+ T cells.

Although TLR agonists (TLRa) can be released from dying normal or malignant human cells, most naturally occurring TLRa are found in bacteria, viruses, and other microorganisms, alerting the immune system to the presence of a pathogen, and activating appropriate defense responses. Activation of TLR signaling results in direct activation of immune cell function as well as indirect activation through induction of secretion of cytokines and chemokines, which act via both autocrine and paracrine mechanisms.

Due to the role of TLRs in host-mediated anti-pathogen and anti-tumor immune responses, significant efforts have been made to produce TLR agonist adjuvants and therapeutics for infection and anti-tumor immunotherapy. A wide variety of mono-specific, purified or synthetic TLR agonists have been produced and tested in pre-clinical and clinical settings. TLR agonists are used as adjuvants in preventative vaccines. However, although anti-pathogen and anti-tumor activity has been observed in the therapeutic vaccine setting, these efforts have encountered significant challenges. Issues encountered have included both lack of potency and excessive toxicity, suggesting that further improvements in preventing and treating existing infections and cancers with TLR agonists are needed.

LPS-endotoxin constitutes about 75% of the gram-negative outer cell membrane and is a potent TLR4 agonist that triggers direct and indirect innate and adaptive immune cell activation in a dose-dependent manner. LPS-endotoxin has been postulated as a major contributor to both the anti-tumor activity and IV toxicity of Gram-negative bacteria.

### SUMMARY

The instant disclosure demonstrates that attenuated, killed, intact and stabilized bacteria produced from non-pathogenic Gram-negative bacterial cells, such as E. coli, exhibited potent efficacy in inhibiting tumor growth and viral replication and activities. At the same time, these treated bacterial cells were well tolerated in in vivo studies, suggesting that they can have a high therapeutic index, suitable for clinical development and use.

Accordingly, one embodiment of the present disclosure provides a method for treating or preventing cancer or an infectious disease in a patient in need thereof, comprising administering to the patient an effective amount of a composition comprising 1×10⁷ to 500×10⁷ intact, stabilized and substantially non-viable E. coli cells which have been treated in such a way as to result in about 70% to 99% reduction of lipopolysaccharide (LPS)-associated endotoxin activity when measured by the Limulus Amebocyte Lysate (LAL) assay as compared to untreated, wild-type E. coli cells, and wherein the composition contains 124 to 62000 endotoxin units (EU) of LPS.

In accordance with one embodiment of the present disclosure, provided is a method for treating or preventing cancer or an infectious disease in a patient in need thereof, comprising administering to the patient an effective amount of a composition comprising 1×10⁷ to 500×10⁷ intact and substantially non-viable E. coli cells which have been treated in such a way as to result in about 70% to 99% reduction of lipopolysaccharide (LPS)-associated endotoxin activity when measured by the Limulus Amebocyte Lysate (LAL) assay as compared to untreated, wild-type E. coli cells, and wherein the composition contains 124 to 62000 endotoxin units (EU) of LPS.

In some embodiments, the composition comprises 2×10⁷ to 200×10⁷ of the intact and substantially non-viable E. coli cells. In some embodiments, the composition comprises 3×10⁷ to 100×10⁷ of the intact and substantially non-viable E. coli cells. In some embodiments, wherein the composition comprises 5×10⁷ to 50×10⁷ of the intact and substantially non-viable E. coli cells. In some embodiments, the composition comprises 3×10⁷, 7×10⁷, 10×10⁷, 20×10⁷, or 70×10⁷ of the intact and substantially non-viable E. coli cells.

In some embodiments, the composition contains 372 EU to 24800 EU of LPS. In some embodiments, the composition contains 372 EU to 8680 EU of LPS. In some embodiments, the composition contains 868 EU to 2480 EU of LPS.

In some embodiments, the intact and substantially non-viable E. coli cells have been treated in such a way as to result in about 85% to 98% reduction of LPS-associated endotoxin. In some embodiments, the intact and substantially non-viable E. coli cells have been treated in such a way as to result in about 90% to 98% reduction of LPS-associated endotoxin.

In some embodiments, the administration is once every day, every other day, every 3 days, every 5 days, every six days, every week, twice per week, three times per week, four times per week, five times per week, six times per week, every 2 weeks, every 3 weeks, every month, every 2 months, every 3 months, every 4 months, every 6 months, every 9 months or every year.

In some embodiments, the treatment of E. coli cells is with polymyxin, preferably polymyxin B or polymyxin E. In some embodiments, the treatment of E. coli cells is at a temperature from about 2° C. to about 10° C., preferably at about 4° C. In some embodiments, the treatment of E. coli cells is with polymyxin and glutaraldehyde. In some embodiments, the treatment is with polymyxin B at a dose range from about 3 mg/mL to about 1,000 mg/mL and with glutaraldehyde at a dose range from about 0.1% to about 1.0%.

In some embodiments, the composition further comprising a phosphate buffer, Mg²⁺, and trehalose. In some embodiments, the composition comprises 0.3×10⁹/mL to 5×10⁹/mL of the intact and substantially non-viable E. coli cells, 0.5 mg/mL to 2 mg/mL of disodium phosphate dihydrate, 0.1 mg/mL to 0.4 mg/mL of monopotassium phosphate, 3 mg/mL to 12 mg/mL of sodium chloride, 0.05 mg/mL to 0.3 mg/mL of potassium chloride, 0.15 mg/mL to 0.6 mg/mL of magnesium chloride hexahydrate, and 50 mg/mL to 200 mg/mL of trehalose dihydrate, and at a pH of 7.0 to 7.7.

In some embodiments, the administration is intravenous, intra-tumoral, subcutaneous, intramuscular, intra-vesical, intra-hepatic, intra-nasal, or peritoneal.

In some embodiments, the patient has a solid tumor. In some embodiments, the solid tumor is a metastatic solid tumor. In some embodiments, the cancer is selected from the group consisting of bladder cancer, gastrointestinal cancer (esophageal, gastric, liver, colorectal, pancreatic) cervical, ovarian, endometrial cancer, leukemia, lymphoma, small cell lung cancer, non-small cell lung cancer, breast cancer, urethral cancer, head and neck cancer, renal cancer, melanoma, prostate cancer and thyroid cancer.

In some embodiments, the method further comprises administering to the patient a second agent selected from the group consisting of cyclophosphamide, IL-2, a non-steroidal anti-inflammatory drug (NSAID), an anti-PD-1 or anti-PD-L1 antibody, an anti-CTLA-4 antibody, and an anti-CD20 antibody.

In some embodiments, the patient has an infection. In some embodiments, the infection is by hepatitis B virus (HBV) or human immunodeficiency virus (HIV).

Also provided, in one embodiment, is a method for providing a therapeutically acceptable composition, comprising: lyophilizing a solution comprising at least 1×10⁶ intact and substantially non-viable E. coli cells which have been treated in such a way as to result in about 70% to 99% reduction of lipopolysaccharide (LPS)-associated endotoxin activity when measured by the Limulus Amebocyte Lysate (LAL) assay as compared to untreated, wild-type E. coli cells, to prepare a lyophilized composition; and storing the lyophilized composition (a) at a temperature of 1° C. to 10° C. for at least 2 months or (b) at a temperature of −15° C. or below for at least 2 years, thereby providing a therapeutically acceptable composition suitable for therapeutic use.

In some embodiments, the solution further comprises a phosphate buffer, Mg²⁺, and trehalose. In some embodiments, the solution comprises 0.3×10⁹/mL to 5×10⁹/mL of the intact and substantially non-viable E. coli cells, 0.5 mg/mL to 2 mg/mL of disodium phosphate dihydrate, 0.1 mg/mL to 0.4 mg/mL of monopotassium phosphate, 3 mg/mL to 12 mg/mL of sodium chloride, 0.05 mg/mL to 0.3 mg/mL of potassium chloride, 0.15 mg/mL to 0.6 mg/mL of magnesium chloride hexahydrate, and 50 mg/mL to 200 mg/mL of trehalose dihydrate, and at a pH of 7.3 to 7.7.

Also provided, in one embodiment, is a method for treating or preventing cancer or an infectious disease in a patient in need thereof, comprising administering to the patient (a) an effective amount of a composition comprising intact and substantially non-viable E. coli cells which have been treated in such a way as to result in about 70% to 99% reduction of lipopolysaccharide (LPS)-associated endotoxin activity when measured by the Limulus Amebocyte Lysate (LAL) assay as compared to untreated, wild-type E. coli cells, and (b) an exogenous antigen associated with the cancer or the infectious disease.

In some embodiments, the antigen is a tumor associated antigen. In some embodiments, the antigen is a viral or bacterial antigen. In some embodiments, the composition comprises 1×10⁷ to 500×10⁷ of the intact and substantially non-viable E. coli cells and contains 124 to 62000 endotoxin units (EU) of LPS.

### BRIEF DESCRIPTION OF THE DRAWINGS

FIG. 1 shows that the manufacturing process of Decoy bacteria stabilizes the bacterial cells.

FIG. 2 shows that Decoy bacteria induced human PBMCs to secrete up to ˜3,500 times higher levels of the same cytokines compared to mouse PBMCs based on 48 hour in vitro (full) dose responses carried out in triplicate.

FIG. 3 shows that Decoy bacteria synergized with human PBMCs to kill human MDA-MB-231 breast carcinoma cells in vitro.

FIG. 4 shows that single agent Decoy inhibited metastasis and extends survival of mice with orthotopic mouse CT26 colorectal carcinoma.

FIG. 5 shows that single agent Decoy bacteria inhibited the in vivo growth of murine colorectal carcinoma in a dose-dependent fashion without significant toxicity.

FIG. 6 shows that Decoy synergized with low dose cyclophosphamide (LDC) to inhibit growth of subcutaneous CT26 colorectal tumors.

FIG. 7 shows that Decoy synergized with low-dose interleukin-2 (IL-2) and/or low-dose indomethacin to inhibit subcutaneous CT26 colorectal carcinoma: treatment was initiated on day 10 with 5 mice per group when tumors were 75 mm³.

FIG. 8 shows that Decoy inhibited the growth of subcutaneous mouse CT26 colorectal carcinoma.

FIG. 9 shows that single agent Decoy extended survival of mice with metastatic mouse Pan02 pancreatic carcinoma.

FIG. 10 shows that Decoy had single agent activity and synergized with oral, low-dose, NSAID indomethacin in a metastatic mouse Pan02 pancreatic carcinoma model.

FIG. 11 shows that Decoy had single agent activity and synergized with NSAID and anti-PD-1 to regress 10/12 of 194 mm³ mouse subcutaneous H22 hepatocellular carcinoma (HCC) tumors.

FIG. 12 shows that cured mice re-challenged with fresh HCC tumor cells rejected the tumors (immunological memory).

FIG. 13 shows that Decoy synergized with NSAID to regress 4/6 183 mm³ mouse subcutaneous H22 hepatocellular carcinoma (HCC) tumors.

FIG. 14 shows that Decoy (1× per week), NSAID and anti-PD-1 synergized to regress 100% of 183 mm³ mouse subcutaneous H22 hepatocellular carcinoma (HCC) tumors.

FIG. 15 shows that Decoy synergized with oral, low-Dose NSAID indomethacin to regress established mouse subcutaneous H22 hepatocellular carcinoma (HCC).

FIG. 16 shows that Decoy and NSAID synergized with anti-PD-1 to regress 200 mm³ mouse H22 hepatocellular carcinoma with a therapeutic index>33 (Decoy groups also received anti-PD-1+ oral NSAID).

FIG. 17 shows that cured mice re-challenged with fresh HCC tumor cells rejected the tumors (immunological memory).

FIG. 18 shows the results of NanoString gene expression analysis of RNA from tumors revealing treatment-related increases in HCC tumor inflammation score (cold to hot tumor).

FIG. 19 shows that Decoy bacteria synergize with low-dose cyclophosphamide (LDC) to induce 100% regressions of established, mouse A20 non-Hodgkin's lymphoma (NHL) after only two weeks of treatment.

FIG. 20 shows synergistic regression of A20 non-Hodgkin's lymphoma (NHL) tumors by Decoy and low-dose cyclophosphamide (LDC) was durable and induced immunological memory.

FIG. 21 shows that A20 Non-Hodgkin's Lymphoma (NHL) tumors that regrow after 1 or 2 weeks of sub-optimal decoy and low-dose cyclophosphamide (LDC) treatment were sensitive to optimal retreatment and that Decoy+LDC treatment can regress very large tumors.

FIG. 22 shows Decoy's synergy with low-dose cyclophosphamide (LDC) to eradicate 200 mm³ subcutaneous mouse A20 non-Hodgkin's lymphoma (NHL) with immunological memory is reproducible.

FIG. 23 shows that high percentage eradication of subcutaneous A20 non-Hodgkin's lymphoma (NHL) by Decoy and low-dose cyclophosphamide (LDC) required CD4+ and CD8+ T cells (adaptive immunity) and NK cells (innate immunity) (but transient regressions and some eradications with single depletions).

FIG. 24 shows that two different Decoy bacterial strains produced similar anti-tumor activity against mouse A20 non-Hodgkin's lymphoma (NHL).

FIG. 25 shows that Decoy and low-dose cyclophosphamide (LDC) synergized with rituximab to induce regression of subcutaneous human Ramos non-Hodgkin's lymphoma (NHL) in SCID mice.

FIG. 26 shows that Decoy and low-dose cyclophosphamide (LDC) can synergize with rituximab to induce immunological memory via the innate immune system.

FIG. 27 shows that expression of a foreign antigen (e.g., HER2, lower panel) significantly improved the anti-tumor effect of single agent Decoy (upper panel). Following single agent Decoy treatment, two of the five animals had complete response (lower panel).

FIG. 28 shows that Decoy inhibited human hepatitis B virus (HBV) replication in a mouse (AAV-HBV) model of chronic HBV infection.

FIG. 29 shows that Decoy inhibited human hepatitis B virus (HBV) HBeAg levels in a mouse (AAV-HBV) model of chronic HBV infection.

FIG. 30 shows that Decoy inhibited human hepatitis B virus (HBV) HBsAg levels in a mouse (AAV-HBV) model of chronic HBV infection.

FIG. 31 shows that Decoy inhibited expression of human hepatitis B Virus (HBV) DNA in livers of mice infected with HBV (AAV-HBV Model).

FIG. 32 shows that Decoy inhibited expression of human hepatitis B virus (HBV) HBeAg in livers of mice (AAV-HBV Model) infected with HBV.

FIG. 33 shows that Decoy and ETV inhibited expression of human hepatitis B virus (HBV) cccDNA-like molecule in livers of mice (AAV-HBV Model) infected with HBV.

FIG. 34 shows that Entecavir and Decoy, as well as the combination, inhibited human hepatitis B virus (HBV) replication in a mouse (AAV-HBV) model of chronic human HBV infection.

FIG. 35 shows that Decoy inhibited human hepatitis B virus (HBV) HBeAg levels in a mouse model of chronic HBV infection.

FIG. 36 shows that Decoy inhibited human hepatitis B virus (HBV) HBsAg levels in a mouse model of chronic HBV infection.

FIG. 37 shows that Decoy reduced human HIV virus levels in a (humanized) mouse model of chronic human HIV infection.

FIG. 38 shows that Decoy induced transient plasma cytokine, chemokine and biomarker expression in human subjects. Reference ranges (healthy volunteers, pg/mL or Units/mL) are provided in the figure. Most baseline measurements reflect lower limits of assay quantitation.

FIG. 39 shows that pharmacokinetic analysis confirmed rapid clearance of systemically-administered Decoy in human subjects.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

### Therapeutic Uses of Treated Bacteria

The experimental examples of the present disclosure demonstrate that attenuated, intact, stabilized and non-viable Gram-negative bacterial cells (e.g., E. coli) treated to significantly reduce LPS-associated endotoxin activity (Decoy bacteria or Decoy) contained agonists of all functional human TLR receptors and receptor heterodimers (TLRs 2, 2/1, 2/6, 3, 4, 5, 7, 8 and 9), as wells as agonists of NOD-like (NLR) and stimulator of interferon (IFN) genes (STING) receptors. Surprisingly, the Decoy bacteria had reduced in vivo pyrogenicity and acute toxicity, but increased ability to induce the secretion of many cytokines and chemokines from immune cells, relative to untreated, parental bacteria. Such treated bacterial cells, therefore, are suitable for providing a safe and effective means to stimulate a subject's immune response, which is useful for treating tumors and bacterial, fungal, parasite or viral infections.

These treated bacterial cells, also referred to as “Decoy bacteria” or simply “Decoy,” are attenuated then 100% killed, stabilized, intact bacteria produced from non-pathogenic, Gram-negative bacterial cells (e.g., E.coli), resulting in ˜90% reduction in LPS-endotoxin activity and pyrogenicity. A comprehensive non-clinical pharmacology program has been developed to support the first in human (FIH) study for Decoy. Primary pharmacodynamic (PD) studies with Decoy included in vitro assessment of induction of cytokine and chemokine secretion by murine and human peripheral blood mononuclear cells and in vivo assessment of IV anti-tumor activity against established, subcutaneous (s.c.) murine colorectal carcinoma, metastatic murine pancreatic carcinoma, established s.c. murine hepatocellular carcinoma (HCC) and established s.c. murine and human non-Hodgkin's lymphoma (NHL) models. Decoy was also tested against established, murine breast carcinoma tumors without and with expression of a foreign antigen. Decoy was tested as a single agent and in combination with certain other therapeutic agents. Significant single agent anti-tumor activity was observed in several models, including regression of established murine breast carcinoma tumors that expressed a foreign antigen. Synergism was observed with low-dose cyclophosphamide (LDC), indomethacin, rituximab, and anti-PD-1 checkpoint therapy, but not with agents such as anti-GITR antibody, INF-g, phenformin, gemcitabine or 5-FU. In addition, the tumor-regressing treatments were associated with induction of immunological memory, as evidenced by rejection of tumor re-challenges in the absence of additional therapy.

Likewise, in animal models, Decoy demonstrated potent activity in inhibiting the replication and activity of hepatitis B viruses (HBV) and human immunodeficiency viruses (HIV), as a single agent or in combination with others, such as entecavir (ETV).

The safety profile of Decoy was determined after 1-hour IV infusion in single-dose, two-week repeat-dose range-finding, and four-week repeat-dose toxicology studies in New Zealand White (NZW) rabbits, which is the non-human laboratory species considered most similar to humans with respect to sensitivity to the adverse effects of LPS. Additional safety information was obtained with Decoy in studies carried out with mice.

The single-dose Decoy maximum tolerated dose (MTD) in the rabbit, with 4 dose levels tested, was determined to be 1.5×10⁹ killed bacteria [KB]/kg. Twice per week Decoy dosing for two weeks, with 4 dose levels tested, produced a no-observed-adverse-effect-level (NOAEL) of 6×10⁷ KB/kg/dose. In the pivotal rabbit 4-week repeat-dose toxicology study (4 dose levels), the NOAEL of Decoy was determined to be 4×10⁷ KB/kg/dose. Decoy was also found to be 97% less pyrogenic in the rabbit (rectal temperature test) than parental (untreated) bacteria and also 3-fold less toxic (acute LD₁₀₀) than parental (untreated) bacteria.

Based on such non-clinical findings, human clinical trials have been designed and conducted, and formulations suitable for clinical use have been developed. An example formulation is shown in Table 4. Example doses are shown in Table 5. The starting dose for the first part of the clinical study is 7×10⁷ KB/patient, which is about 1/10 of the human equivalent dose (HED) determined from the no-observed-adverse-effect-level (NOAEL) observed in the twice per week 4-week rabbit toxicology study (4×10⁷ KB/kg dose). Based on the 4-week Good Laboratory Practice (GLP) study data, including a 3.1-fold allometric scaling factor (dose decrease) to the HED plus a 10-fold dose decrease safety adjustment, the starting human dose would be 1.29×10⁶ KB per kg or approximately 16 Decoy-associated endotoxin units (EU)/kg, which is 7.74×10⁷ KB per 60 kg subject; equivalent to approximately 960 EU per 60 kg subject (not the conventional 70 kg, in order to account for a lower patient weight). The starting dose in the study will be slightly lower, at 7.0×10¹⁰ KB: equivalent to 868 EU per 60 kg subject or 1.8 ng/kg LPS. Since rapid clearance of Decoy by the liver and spleen is anticipated (within minutes to about 1 hour) based on published results of systemic clearance of live and killed bacteria in mice, rabbits and humans, dose adjustment based on body weight is not considered to be necessary. The LPS in the starting dose in the study is lower than the highest dose (4 ng/kg) determined to be well-tolerated after IV administration of purified LPS to over 1,000 healthy human volunteers. In later parts of the clinical study, repeated weekly doses will be administered. If there is no safety concern, the subject will receive continuous weekly dosing of Decoy for up to 2 years.

As shown in the preliminary results from the Phase I clinical trial (Example 4), a one-hour intravenous infusion of 7×10⁷ killed Decoy bacteria resulted in stable disease in all four cancer patients, including three in which the tumor was progressing prior to the treatment. Also important, the Decoy bacteria were cleared from blood within 30-120 minutes after the end of the infusion and produced transient induction in plasma of over 50 cytokines, chemokines and biomarkers, many of which are known to participate directly in stimulation of innate and/or adaptive immune responses, including anti-tumor responses. Transient induction of the cytokines and chemokines is an important and novel feature of the response to Decoy bacteria, that helps to reduce the possibility of systemic toxicities that are known to result from continuous or long-term systemic exposure to these potent immune-activating molecules.

In accordance with one embodiment of the present disclosure, therefore, provided is a method for treating or preventing cancer in a patient in need thereof, which method entails administering to the patient an effective amount of a composition of treated bacteria.

In another embodiment, provided is a method of stimulating an immune response in a subject in need thereof. In another embodiment, provided is a method for preventing or treating an infection in a patient in need thereof. In another embodiment, provided is a method for treating immunodeficiency in a patient in need thereof. In another embodiment, a method of vaccinating a subject at risk of infection or cancer is provided.

The treated bacterial cells, in some embodiments, are intact, stabilized and substantially non-viable Gram-negative bacterial cells which have been treated to reduce lipopolysaccharide (LPS)-associated endotoxin activity and/or pyrogenicity. In some embodiments, the intact and substantially non-viable Gram-negative bacterial cells have been treated in such a way as to result in about 70% to 99% reduction of lipopolysaccharide (LPS)-associated endotoxin activity when measured by the Limulus Amebocyte Lysate (LAL) assay as compared to untreated, wild-type Gram-negative bacterial.

Candidate bacterial organisms that may be employed by the methods herein are Gram-negative and which include those that have LPS-associated endotoxin activity as wild-type organisms. The term “Gram-negative bacteria” refers to bacteria that do not retain the initial basic dye stain (e.g., crystal violet) that is part of the procedure known as the Gram stain. In an exemplary Gram stain, cells are first fixed to a slide by heat and stained with a basic dye (e.g., crystal violet), which is taken up by both Gram-negative and Gram-positive bacteria. The slides are then treated with a mordant (e.g., Gram's iodine), which binds to basic dye (e.g. crystal violet) and traps it in the cell. The cells are then washed with acetone or alcohol, and then counterstained with a second dye of different color (e.g., safranin). Gram-positive organisms retain the initial violet stain, while Gram-negative organisms are decolorized by the wash solvent organic and hence show the counterstain. Exemplary Gram-negative bacteria include, but are not limited to, Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Corynebacteria spp., Citrobacter spp., Chlamydia spp., Brucella spp., Pseudomonas spp., Helicobacter spp. and Vibrio spp.

Within gram-negative organisms are the Enterobacteriaceae, a large family that includes, along with many harmless symbionts, many well-known pathogens, such as Salmonella, E. coli, Yersinia pestis, Klebsiella and Shigella, Proteus, Enterobacter, Serratia, and Citrobacter. Members of the Enterobacteriaceae have been referred to as enterobacteria, as several members live in the intestines of animals.

In one embodiment, E. coli is selected as the organism. One particular strain contemplated is E. coli strain 2617-143-312, (Migula) Castellani and Chalmers (ATCC® 13070™). An additional E. coli strain which may be used includes MG1655 (ATCC® 47076).

The term “Lipopolysaccharide” (LPS) refers to large molecules consisting of a lipid and a polysaccharide (glycophospholipid) joined by a covalent bond. LPS comprises three parts: 1) O antigen; 2) Core oligosaccharide, and 3) Lipid A. The O-antigen is a repetitive glycan polymer attached to the core oligosaccharide and comprises the outermost domain of the LPS molecule. Core oligosaccharide attaches directly to lipid A and commonly contains sugars such as heptose and 3-deoxy-D-mannooctulosonic acid (also known as KDO, keto-deoxyoctulosonate). Lipid A is a phosphorylated glucosamine disaccharide linked to multiple fatty acids. The fatty acids anchor the LPS into the bacterial outer membrane, and the rest of the LPS projects from the cell surface.

Endotoxin activity resides in the lipid A domain portion of LPS, and thus is also referred to as “LPS-associated endotoxin activity or LPS-endotoxin activity.” Gram-negative bacteria contain additional TLRa, including agonists of TLR2/1, 2/6, 2, 3, 5, 7, 8 and 9. as well as other immune stimulating molecules, such as STING (stimulator of interferon genes) and NOD (nucleotide-binding oligomerization domain-containing protein) agonists. Intact bacteria that enter the circulatory system are rapidly engulfed by immune cells in the liver and spleen, leading to direct immune cell/pathway activation and indirect immune cell/pathway activation via induction of cytokine and chemokine secretion. Rapid clearance of circulating bacterial cells by immune cells in the liver and spleen helps to localize immune activation to key immune organs. Significant amounts of LPS-endotoxin are released or shed by live, proliferating bacteria. If live bacterial cells proliferate, invade normal tissues and cells and/or break down in the circulation, large amounts of immune activators can be released systemically. This can produce inappropriate and excessive inflammatory responses throughout the body, leading to potentially fatal shock (called endotoxic or septic shock). Thus, the invention and use of killed and stabilized bacteria with reduced LPS-endotoxin activity should allow for short-lived or transient immune activation in the liver and spleen after systemic administration, while significantly reducing the potential for inappropriate systemic inflammation that can be produced by live bacteria able to invade and proliferate in normal cells/tissues, proliferate and break down in the systemic circulation and shed or release many different types of immune activators throughout the body. The most potent bacteria-associated immune stimulator, which can contribute to both anti-tumor and anti-viral efficacy as well as systemic toxicity is LPS-endotoxin. LPS-associated endotoxin activity can be measured by methods well known in the art, including, for example, the Limulus Amebocyte Lysate (LAL) assay, which utilizes blood from the horseshoe crab, and can detect very low levels of LPS. The presence of endotoxin activity will result in coagulation of the limulus blood lysate due to amplification via an enzymatic cascade. Gel clotting, turbidometric, and chromogenic forms of the LAL assay are commercially available.

Enzyme linked immunoadsorbent assay (ELISA)-based endotoxin activity assays are also known such as the EndoLISA® from Hyglos, Munich area of Germany. This assay employs an LPS specific phage protein attached to the solid phase to capture LPS, and following a wash step, the presence of LPS is determined by addition of recombinant Factor C, which when activated by LPS, cleaves a compound that then emits fluorescence. Factor C, present in the Limulus amebocyte lysate, normally exists as a zymogen, and is the primer of the coagulation cascade that occurs in the LAL test.

Pyrogenicity refers to the ability of an agent to cause fever in a subject. Pyrogenicity can be measured as rectal temperature increase in rabbits in response to intravenously administered TLR agonists, organisms or derivatives thereof.

Various methods are available to reduce the endotoxin activity and/or pyrogenicity of Gram-negative organisms. The methods include treatment of the organisms with an agent that binds to LPS or disrupts its formation.

In one embodiment, reduction in endotoxin activity or pyrogenicity is achieved by treating the bacterial organisms with an antibiotic that inactivates endotoxin. A suitable such antibiotic is polymyxin, including polymyxin B or polymyxin E. It is within the skill of one in the art to determine the amount of antibiotic and conditions for treatment. In one embodiment, the polymyxin, either polymyxin B or E, may be employed at a concentration of approximately 3 micrograms to 5,000 micrograms per milliliter. In another embodiment, the concentration of polymyxin may be from about 200 micrograms to 5,000 micrograms per milliliter. In one embodiment, the antibiotic is applied to the bacteria for 10 minutes to 4 hours or from about 30 minutes to about 3 hours.

In one embodiment, the bacteria are grown in the presence of magnesium (Mg) in the form of MgCl₂. In one embodiment, the bacteria are treated with polymyxin in the presence of MgCl₂, as well as at a temperature suitable to maintain the bacteria's integrity. In one embodiment, the concentration of MgCl₂ in the growth medium is from about 0.5 mM to about 5.0 mM, or about 2 mM, and the concentration of MgCl₂ in the treatment medium is from about 5.0 mM to about 30 mM, or about 20 mM. In one embodiment, the temperature of the treatment medium is from about 2° C. to about 10° C., or about 4° C. Bacterial integrity is determined by efficiency of recovery in a well-defined pellet after centrifugation at 3,000×g for 10 minutes, and by electron microscopy or optical microscopy with Gram staining. In a preferred embodiment, bacterial recovery after treatment and wash is greater than about 80% and the bacteria appear intact by optical or electron microscopy.

In another embodiment, reduction in endotoxin activity is achieved by treating the bacterial organisms with an antibiotic known to disrupt the biosynthesis of KDO2-Lipid IV_{A}. For example, Goldman et al., J Bacteriol. 170(5):2185-91, 1988 describe antibacterial agents, including antibacterial agent III, which specifically inhibit CTP:CMP-3-deoxy-D-manno-octulosonate cytidylyltransferase activity and which are useful to block the incorporation of 3-deoxy-D-manno-octulosonate (KDO) into LPS of Gram-negative organisms. As LPS synthesis ceased, bacterial growth ceased. The addition of KDO to LPS precursor species lipid IV_{A} is the major pathway of lipid A-KDO formation in both S. typhimurium and E. coli. In one embodiment, the antibiotic is antibacterial agent III and Gram-negative bacteria are treated with a suitable amount, such as, for example 5 micrograms per milliliter to 500 micrograms per milliliter for a suitable time, for example 2 to 8 hours.

Likewise, the compound alpha-C-(1,5-anhydro-7-amino-2,7-dideoxy-D-manno-heptopyranosyl)-carboxylate is known to inhibit 3-deoxy-D-manno-octulosonate cytidylytransferase (CMP-KDO synthetase), a cytoplasmic enzyme which activates 3-deoxy-D-manno-octulosonate (KDO) for incorporation into LPS (Nature. 1987 10-16;329(6135):162-4). Therefore, treatment of the organisms with the compound can reduce LPS-associated endotoxin activity as well.

In another embodiment, reduction in endotoxin activity is achieved by treating the organisms with an LPS inhibitor. For instance, a bacterial cyclic lipopeptide, surfactin, was shown to bind to lipid A, suppressing its activity (J Antibiot 2006 59(1):35-43).

In addition to LPS-associated endotoxin, various other constituents of Gram-negative organisms can induce or contribute to pyrogenicity and septic shock, including outer membrane proteins, fimbriae, pili, lipopeptides, and lipoproteins (reviewed by Jones, M., Int. J. Pharm. Compd., 5(4):259-263, 2001). Pyrogenicity can be measured by a rabbit method, well known in the art, involving assessment of rectal temperature after intravenous administration of putative pyrogens.

It has been found that treatment of a Gram-negative organism with a combination of polymyxin B and glutaraldehyde produced a 30-fold reduction in pyrogenicity, as measured in rabbits. In one embodiment, 1,000 micrograms per milliliter (μg/mL) of polymyxin B and 1% glutaraldehyde was employed to produce a 30-fold reduction in pyrogenicity, as measured in rabbits. The pyrogenicity is reduced by a combination of polymyxin B reaction with LPS and glutaraldehyde reactivity with LPS and other bacterial constituents. The bi-functional chemical cross-linking activity of glutaraldehyde also serves to kill and stabilize the bacterial cells, thus serving a triple role in this setting (reduction of pyrogenic activity, cell killing and cell stabilization).

Thus, in one embodiment is provided a method of reducing endotoxin activity and pyrogenicity of and killing and stabilizing a Gram-negative bacterial microorganism by treating said bacteria with a combination of 1,000 μg/mL polymyxin B and 1% glutaraldehyde. In another embodiment, the Gram-negative bacteria are treated with a combination of polymyxin B at a dose range between about 3 μg/mL to about 1,000 μg/mL and glutaraldehyde at a dose range between about 0.1% to about 1.0%. In a further embodiment, the dose range of polymyxin B is between about 100 μg/mL to about 1,000 μg/mL and glutaraldehyde is at a dose range between about 0.25% to about 1.0%. Additionally, Gram-negative bacteria may be treated, for example with a dose range of polymyxin B between about 1,000 μg/mL to about 3,000 μg/mL and glutaraldehyde is at a dose range between about 0.25% to about 1.0%. In another aspect, Gram-negative bacteria maybe treated, for example with a dose range of polymyxin B between about 3,000 μg/mL to about 5,000 μg/mL and glutaraldehyde is at a dose range between about 0.25% to about 2.0%.

In some embodiments, the intact and substantially non-viable Gram-negative bacterial cells have at least about 70% reduction of LPS-associated endotoxin activity (e.g., as measured by the LAL assay) as compared to untreated, wild-type bacteria. In some embodiments, the reduction is at least about 70%, 75%, 80%, 85%, 90%, 91%, 92%, 93%, 94%, 95%, 96%, 97%, 98%, 99%, 99.5%, 99.9%, 99.95% or 99.98%. In some embodiments, the reduction is not greater than about 95%, 96%, 97%, 98%, 99%, 99.5%, 99.9%, 99.95%, 99.98% or 99.99%. In some embodiments, the reduction is from about 70% to about 99.99%, from about 80% to about 99.99%, from about 90% to about 99.5% or 99%, from about 91% to about 99%, from about 92% to about 98%, from about 93% to about 97%, from about 94% to about 96%, from about 94.5% to about 95.5%, from about 94% to about 97%, from about 95% to about 98%, from about 96% to about 99%, from about 97% to about 99.5%, or from about 98% to about 99.9%, without limitation.

In some embodiments, certain residual active LPS levels are preferred. For instance, in some embodiments, in a composition of the present disclosure, there is about 1 to 200 ng active LPS per 1×10⁸ cells. In some embodiments, there is about 2 to 200 ng, about 5 to 150 ng, about 5 to 120 ng, about 10 to 120 ng, about 20 to 100 ng, about 20 to 50 ng, about 10 to 50 ng active LPS per 1×10⁸ cells.

In some embodiments, the intact and substantially non-viable Gram-negative bacterial cells have at least about 70% reduction of pyrogenicity (e.g., as measured by in vivo rabbit assay) as compared to untreated wild-type bacteria. In some embodiments, the reduction is at least about 70%, 75%, 80%, 85%, 90%, 91%, 92%, 93%, 94%, 95%, 96%, 97%, 98%, 99%, 99.5%, 99.9%, 99.95% or 99.98%. In some embodiments, the reduction is not greater than about 95%, 96%, 97%, 98%, 99%, 99.5%, 99.9%, 99.95%, 99.98% or 99.99%. In some embodiments, the reduction is from about 70% to about 99.99%, from about 80% to about 99.99%, from about 90% to about 99.5% or 99%, from about 91% to about 99%, from about 92% to about 98%, from about 93% to about 97%, from about 94% to about 96%, from about 94.5% to about 95.5%, from about 94% to about 97%, from about 95% to about 98%, from about 96% to about 99%, from about 97% to about 99.5%, or from about 98% to about 99.9%, without limitation.

As provided above, in addition to LPS-associated endotoxin, various other constituents of Gram-negative organisms can also induce or contribute to pyrogenicity, such as outer membrane proteins, fimbriae, pili, lipopeptides, and lipoproteins. In some embodiments, the intact and substantially non-viable Gram-negative bacterial cells are treated in a manner such that the reduction of pyrogenicity is achieved by both reduction of LPS-associated endotoxin activity and reduction of non-LPS-associated pyrogenicity, such as inactivation, removal or blocking of outer membrane proteins, fimbriae, pili, lipopeptides, or lipoproteins. In some embodiments, the reduction of non-LPS-associated pyrogenicity is at least about 50%, 60%, 70%, 75%, 80%, 85%, 90%, 91%, 92%, 93%, 94%, 95%, 96%, 97%, 98%, 99%, 99.5%, 99.9%, 99.95% or 99.98%. In some embodiments, the reduction is not greater than about 60%, 70%, 75%, 80%, 85%, 90%, 91%, 92%, 93%, 94%, 95%, 96%, 97%, 98%, 99%, 99.5%, 99.9%, 99.95%, 99.98% or 99.99%.

Bacteria for administration according to the methods of the disclosure are rendered non-viable or substantially non-viable either prior to administration or become so upon administration. What is meant by “non-viable” is that the organisms are killed by treatment with an exogenous agent, and/or contain a mutation that results in an inability of the organisms to survive in a mammalian host. Substantially non-viable bacteria are strains that have had their viability reduced by at least 80%, 85%, 90%, 95%, 99%, or more.

Bacteria can be made non-viable by treating with a compound such as polymyxin. Polymyxin binds to LPS and interferences with membrane integrity as the bacteria divide, with viability being reduced as a result of permeabilization of the cell envelope. If viability is reduced by this method, steps need be taken to prevent cell lysis and keep the cells intact. Another approach is to grow bacterial strains with conditional mutations in the LPS biosynthesis pathway that are suppressed during growth and then transfer to a non-permissive condition which activates the mutation and disrupts LPS biosynthesis. In each instance, the procedure applied is one that renders the bacteria non-viable by, determining in each setting, the optimal time of treatment or dose of compound, such that viability has been substantially lost with retention of significant bacterial cell integrity. In the case where non-viability is less than 100%, bacteria can be used which contain a mutation preventing further proliferation of viable bacteria in a mammalian host (e.g. a diaminopimelic acid auxotroph, as described by Bukhari and Taylor, J. Bacteriol. 105(3):844-854, 1971 and Curtiss et al., Immunol. Invest. 18(1-4):583-596, 1989).

### Dosage and Dosing Schedule

Suitable effective amounts (doses) and dosing schedules are also determined for the therapeutic and prophylactic uses. In this context, a preferred Gram-negative bacterial species is E. coli. In some embodiments, an effective amount of the treated E. coli cells includes 1×10⁷ to 500×10⁷ of the intact and substantially non-viable E. coli cells.

As demonstrated in the accompanying examples, the Decoy product is highly potent against tumors and viral infections, in particular when used in the presence of immune cells, or in combination with certain other therapeutic agents, such as cyclophosphamide, IL-2, a non-steroidal anti-inflammatory drug (NSAID) such as indomethacin, an anti-PD-1 or anti-PD-L1 antibody, an anti-CTLA-4 antibody, and an anti-CD20 antibody (e.g., rituximab). Accordingly, the effective amount can be as low as (or greater than) 1×10⁷ treated cells, or alternatively as low as (or greater than) 2×10⁷, 3×10⁷, 4×10⁷, 5×10⁷, 7×10⁷, 7.74×10⁷, 10×10⁷, 15×10⁷, 20×10⁷, 30×10⁷, 40×10⁷, 50×10⁷, 60×10⁷, 70×10⁷, 80×10⁷, 90×10⁷, 100×10⁷, 150×10⁷, 200×10⁷, 250×10⁷, 300×10⁷, or 400×10⁷ treated cells.

Also as demonstrated, the Decoy product is sufficiently safe to allow the clinical use of high doses. Accordingly, the effective amount can be as high as (or lower than) 500×10⁷ treated cells, or alternatively as high as (or lower than) 2×10⁷, 3×10⁷, 4×10⁷, 5×10⁷, 7×10⁷, 7.74×10⁷, 10×10⁷, 15×10⁷, 20×10⁷, 30×10⁷, 40×10⁷, 50×10⁷, 60×10⁷, 70×10⁷, 80×10⁷, 90×10⁷, 100×10⁷, 150×10⁷, 200×10⁷, 250×10⁷, 300×10⁷, or 400×10⁷ treated cells.

In some embodiments, the effective amount is from 1×10⁷ to 500×10⁷ treated cells. In some embodiments, the effective amount is from 1×10⁷ to 400×10⁷, from 1×10⁷ to 300×10⁷, from 1×10⁷ to 200×10⁷, from 1×10⁷ to 150×10⁷, from 1×10⁷ to 100×10⁷, from 1×10⁷ to 70×10⁷, from 1×10⁷ to 50×10⁷, from 1×10⁷ to 20×10⁷, or from 1×10⁷ to 10×10⁷ treated cells. In some embodiments, the effective amount is from 3×10⁷ to 400×10⁷, from 3×10⁷ to 300×10⁷, from 3×10⁷ to 200×10⁷, from 3×10⁷ to 150×10⁷, from 3×10⁷ to 100×10⁷, from 3×10⁷ to 70×10⁷, from 3×10⁷ to 50×10⁷, from 3×10⁷ to 20×10⁷, or from 3×10⁷ to 10×10⁷ treated cells. In some embodiments, the effective amount is from 7×10⁷ to 400×10⁷, from 7×10⁷ to 300×10⁷, from 7×10⁷ to 200×10⁷, from 7×10⁷ to 150×10⁷, from 7×10⁷ to 100×10⁷, from 7×10⁷ to 70×10⁷, from 7×10⁷ to 50×10⁷, from 7×10⁷ to 20×10⁷, or from 7×10⁷ to 10×10⁷ treated cells. In some embodiments, the effective amount is from 7.74×10⁷ to 400×10⁷, from 7.74×10⁷ to 300×10⁷, from 7.74×10⁷ to 200×10⁷, from 7.74×10⁷ to 150×10⁷, from 7.74×10⁷ to 100×10⁷, from 7.74×10⁷ to 70×10⁷, from 7.74×10⁷ to 50×10⁷, from 7.74×10⁷ to 20×10⁷, or from 7.74×10⁷ to 10×10⁷ treated cells.

In some embodiments, the effective amount is from 10×10⁷ to 400×10⁷, from 10×10⁷ to 300×10⁷, from 10×10⁷ to 200×10⁷, from 10×10⁷ to 150×10⁷, from 10×10⁷ to 100×10⁷, from 10×10⁷ to 70×10⁷, from 10×10⁷ to 50×10⁷, or from 10×10⁷ to 20×10⁷ treated cells. In some embodiments, the effective amount is from 15×10⁷ to 400×10⁷, from 15×10⁷ to 300×10⁷, from 15×10⁷ to 200×10⁷, from 15×10⁷ to 150×10⁷, from 15×10⁷ to 100×10⁷, from 15×10⁷ to 70×10⁷, from 15×10⁷ to 50×10⁷, or from 15×10⁷ to 20×10⁷ treated cells. In some embodiments, the effective amount is from 20×10⁷ to 400×10⁷, from 20×10⁷ to 300×10⁷, from 20×10⁷ to 200×10⁷, from 20×10⁷ to 150×10⁷, from 20×10⁷ to 100×10⁷, from 20×10⁷ to 70×10⁷, from or 20×10⁷ to 50×10⁷ treated cells.

In some embodiments, the effective amount is from 30×10⁷ to 400×10⁷, from 30×10⁷ to 300×10⁷, from 30×10⁷ to 200×10⁷, from 30×10⁷ to 150×10⁷, from 30×10⁷ to 100×10⁷, from 30×10⁷ to 70×10⁷, or from 30×10⁷ to 50×10⁷ treated cells. In some embodiments, the effective amount is from 40×10⁷ to 400×10⁷, from 40×10⁷ to 300×10⁷, from 40×10⁷ to 200×10⁷, from 40×10⁷ to 150×10⁷, from 40×10⁷ to 100×10⁷, from 40×10⁷ to 70×10⁷, or from 40×10⁷ to 50×10⁷ treated cells. In some embodiments, the effective amount is from 50×10⁷ to 400×10⁷, from 50×10⁷ to 300×10⁷, from 50×10⁷ to 200×10⁷, from 50×10⁷ to 150×10⁷, from 50×10⁷ to 100×10⁷, or from 50×10⁷ to 70×10⁷ treated cells. In some embodiments, the effective amount is from 70×10⁷ to 400×10⁷, from 70×10⁷ to 300×10⁷, from 70×10⁷ to 200×10⁷, from 70×10⁷ to 150×10⁷, or from 70×10⁷ to 100×10⁷ treated cells. In some embodiments, the effective amount is from 100×10⁷ to 400×10⁷, from 100×10⁷ to 300×10⁷, from 100×10⁷ to 200×10⁷, or from 100×10⁷ to 150×10⁷ treated cells.

In some embodiments, the effective amount is about 1 (or 0.5˜1.5)×10⁷, 2 (or 1.5˜2.5)××10⁷, 3 (or 2˜4)×10⁷, 4 (or 3˜5)×10⁷, 5 (or 4˜6)×10⁷, 7 (or 6˜8)×10⁷, 7.74×10⁷, 10 (or 8˜12)×10⁷, 15 (or 13˜17)×10⁷, 20 (or 15˜25)×10⁷, 30 (or 25˜35)×10⁷, 40 (or 30˜50)×10⁷, 50 (or 40˜60)×10⁷, 60 (or 50˜70)×10⁷, 70 (or 60˜80)×10⁷, 80 (or 70˜90)×10⁷, 90 (or 80˜100)×10⁷, 100 (or 80˜120)×10⁷, 150 (or 130˜170)×10⁷, 200 (or 150˜250)×10⁷, 250 (or 200˜300)×10⁷, 300 (or 200˜400)×10⁷, or 400 (or 300˜500)×10⁷ treated cells.

In some embodiments, the effective amount is at least, for each kilogram (kg) of body weight of the patient, 0.02×10⁷ treated cells. In some embodiments, the effective amount is at least, for each kilogram (kg) of body weight of the patient, 0.05×10⁷, 0.12×10⁷, 0.13×10⁷, 0.17×10⁷, 0.33×10⁷, 0.83×10⁷, 1.17×10⁷, 1.67×10⁷, 2.50×10⁷, 3.33×10⁷, 5.00×10⁷, or 6.67×10⁷ treated cells. In some embodiments, the effective amount is no more than, for each kilogram (kg) of body weight of the patient, 0.05×10⁷, 0.12×10⁷, 0.13×10⁷, 0.17×10⁷, 0.33×10⁷, 0.83×10⁷, 1.17×10⁷, 1.67×10⁷, 2.50×10⁷, 3.33×10⁷, 5.00×10⁷, 6.67×10⁷, or 8.33×10⁷ treated cells.

In some embodiments, the effective amount of the treated cells include a predetermined amount of active LPS, which can be measured with endotoxin units (EU). In some embodiments, the effective amount contains 124 to 62000 endotoxin units (EU) of LPS. In some embodiments, the effective amount contains at least 124, 372, 868, 960, 1240, 2480, 6200, 8680, 12400, 18600, 24800, 37200 or 49600 EU of LPS. In some embodiments, the effective amount contains no more than 62000 endotoxin units (EU) of LPS. In some embodiments, the effective amount contains no more than 372, 868, 960, 1240, 2480, 6200, 8680, 12400, 18600, 24800, 37200 or 49600 EU of LPS.

In some embodiments, the effective amount contains 124 to 62000 endotoxin units (EU) of LPS. In some embodiments, the effective amount contains 372 to 62000 EU of LPS, or alternatively 868 to 62000 EU, 960 to 62000 EU, 1240 to 62000 EU, 2480 to 62000 EU, 6200 to 62000 EU, 8680 to 62000 EU, 12400 to 62000 EU, 18600 to 62000 EU, 24800 to 62000 EU, 37200 to 62000 EU, or 49600 to 62000 EU of LPS. In some embodiments, the effective amount contains 372 to 49600 EU, 868 to 49600 EU, 960 to 49600 EU, 1240 to 49600 EU, 2480 to 49600 EU, 6200 to 49600 EU, 8680 to 49600 EU, 12400 to 49600 EU, 18600 to 49600 EU, 24800 to 49600 EU, or 37200 to 49600 EU of LPS. In some embodiments, the effective amount contains 372 to 37200 EU, 868 to 37200 EU, 960 to 37200 EU, 1240 to 37200 EU, 2480 to 37200 EU, 6200 to 37200 EU, 8680 to 37200 EU, 12400 to 37200 EU, 18600 to 37200 EU, or 24800 to 37200 EU of LPS.

In some embodiments, the effective amount contains 372 to 24800 EU, 868 to 24800 EU, 960 to 24800 EU, 1240 to 24800 EU, 2480 to 24800 EU, 6200 to 24800 EU, 8680 to 24800 EU, 12400 to 24800 EU, or 18600 to 24800 EU of LPS. In some embodiments, the effective amount contains 372 to 18600 EU, 868 to 18600 EU, 960 to 18600 EU, 1240 to 18600 EU, 2480 to 18600 EU, 6200 to 18600 EU, 8680 to 18600 EU, or 12400 to 18600 EU of LPS. In some embodiments, the effective amount contains 372 to 12400 EU, 868 to 12400 EU, 960 to 12400 EU, 1240 to 12400 EU, 2480 to 12400 EU, 6200 to 12400 EU, or 8680 to 12400 EU of LPS. In some embodiments, the effective amount contains 372 to 8680 EU, 868 to 8680 EU, 960 to 8680 EU, 1240 to 8680 EU, 2480 to 8680 EU, or 6200 to 8680 EU of LPS. In some embodiments, the effective amount contains 372 to 6200 EU, 868 to 6200 EU, 960 to 6200 EU, 1240 to 6200 EU, or 2480 to 6200 EU of LPS. In some embodiments, the effective amount contains 372 to 6200 EU, 868 to 2480 EU, 960 to 2480 EU, or 1240 to 2480 EU of LPS. In some embodiments, the effective amount contains 372 to 1240 EU, 868 to 1240 EU, or 960 to 1240 EU of LPS. In some embodiments, the effective amount contains 372 to 960 EU or 868 to 960 EU of LPS. In some embodiments, the effective amount contains 372 to 868 EU.

In some embodiments, the effective amount, for each kilogram (kg) of body weight of the patient, contains at least 2.07 endotoxin units (EU) of LPS. In some embodiments, the effective amount, for each kilogram (kg) of body weight of the patient, contains at least 6.20, 14.47, 16.00, 20.67, 41.33, 103.33, 144.67, 206.67, 310.00, 413.33, 620.00, or 826.67 endotoxin units (EU) of LPS. In some embodiments, the effective amount, for each kilogram (kg) of body weight of the patient, contains no more than 6.20, 14.47, 16.00, 20.67, 41.33, 103.33, 144.67, 206.67, 310.00, 413.33, 620.00, 826.67 or 1033.33 endotoxin units (EU) of LPS.

In some embodiments, the effective amount of the treated cells include a predetermined amount of active LPS, which can be measured with the amount of active LPS. In some embodiments, the effective amount contains 15 ng to 7714 ng active LPS. In some embodiments, the effective amount contains at least 15 ng active LPS, or at least 46, 108, 119, 154, 309, 771, 1080, 1543, 2314, 3086, 4629, or 6171 ng active LPS. In some embodiments, the effective amount contains no more than 46, 108, 119, 154, 309, 771, 1080, 1543, 2314, 3086, 4629, 6171 or 7714 ng active LPS.

The term “active LPS” refers to the LPS in a composition that is able to exhibit LPS-associated endotoxin activity, e.g., as measured by the LAL assay, where 5-9 endotoxin units (EU) are considered to be equivalent to 1 ng of active LPS, based on a standard LPS preparation. The amount of active LPS in a composition can be described as the weight of uninhibited LPS that is able to exhibit the same level of LPS-associated endotoxin activity as the composition.

In some embodiments, the effective amount contains from 15 ng to 7714 ng active LPS. In some embodiments, the effective amount contains from 46 ng to 7714 ng, 108 ng to 7714 ng, 119 ng to 7714 ng, 154 ng to 7714 ng, 309 ng to 7714 ng, 771 ng to 7714 ng, 1080 ng to 7714 ng, 1543 ng to 7714 ng, 2314 ng to 7714 ng, 3086 ng to 7714 ng, 4629 ng to 7714 ng, or 6171 ng to 7714 ng active LPS. In some embodiments, the effective amount contains from 15 ng to 6171 ng, 46 ng to 6171 ng, 108 ng to 6171 ng, 119 ng to 6171 ng, 154 ng to 6171 ng, 309 ng to 6171 ng, 771 ng to 6171 ng, 1080 ng to 6171 ng, 1543 ng to 6171 ng, 2314 ng to 6171 ng, 3086 ng to 6171 ng, or 4629 ng to 6171 ng active LPS. In some embodiments, the effective amount contains from 15 ng to 4629 ng, 46 ng to 4629 ng, 108 ng to 4629 ng, 119 ng to 4629 ng, 154 ng to 4629 ng, 309 ng to 4629 ng, 771 ng to 4629 ng, 1080 ng to 4629 ng, 1543 ng to 4629 ng, 2314 ng to 4629 ng, or 3086 ng to 4629 ng active LPS.

In some embodiments, the effective amount contains from 15 ng to 3086 ng, 46 ng to 3086 ng, 108 ng to 3086 ng, 119 ng to 3086 ng, 154 ng to 3086 ng, 309 ng to 3086 ng, 771 ng to 3086 ng, 1080 ng to 3086 ng, 1543 ng to 3086 ng, or 2314 ng to 3086 ng active LPS. In some embodiments, the effective amount contains from 15 ng to 2314 ng, 46 ng to 2314 ng, 108 ng to 2314 ng, 119 ng to 2314 ng, 154 ng to 2314 ng, 309 ng to 2314 ng, 771 ng to 2314 ng, 1080 ng to 2314 ng, or 1543 ng to 2314 ng active LPS. In some embodiments, the effective amount contains from 15 ng to 1543 ng, 46 ng to 1543 ng, 108 ng to 1543 ng, 119 ng to 1543 ng, 154 ng to 1543 ng, 309 ng to 1543 ng, 771 ng to 1543 ng, or 1080 ng to 1543 ng active LPS.

In some embodiments, the effective amount contains from 15 ng to 1080 ng, 46 ng to 1080 ng, 108 ng to 1080 ng, 119 ng to 1080 ng, 154 ng to 1080 ng, 309 ng to 1080 ng, or 771 ng to 1080 ng active LPS. In some embodiments, the effective amount contains from 15 ng to 771 ng, 46 ng to 771 ng, 108 ng to 771 ng, 119 ng to 771 ng, 154 ng to 771 ng, or 309 ng to 771 ng active LPS. In some embodiments, the effective amount contains from 15 ng to 309 ng, 46 ng to 309 ng, 108 ng to 309 ng, 119 ng to 309 ng, or 154 ng to 309 ng active LPS. In some embodiments, the effective amount contains from 15 ng to 154 ng, 46 ng to 154 ng, 108 ng to 154 ng, or 119 ng to 154 ng active LPS. In some embodiments, the effective amount contains from 15 ng to 119 ng, 46 ng to 119 ng, or 108 ng to 119 ng active LPS. In some embodiments, the effective amount contains from 15 ng to 46 ng active LPS.

In some embodiments, the effective amount, for each kilogram (kg) bodyweight of the patient, contains at least 0.26 ng active LPS. In some embodiments, the effective amount, for each kilogram (kg) bodyweight of the patient, contains at least 0.77, 1.80, 1.99, 2.57, 5.14, 12.86, 18.00, 25.71, 38.57, 51.43, 77.14, or 102.86 ng active LPS. In some embodiments, the effective amount, for each kilogram (kg) bodyweight of the patient, contains no more than 0.77, 1.80, 1.99, 2.57, 5.14, 12.86, 18.00, 25.71, 38.57, 51.43, 77.14, 102.86 or 128.57 ng active LPS.

In some embodiments, the administration of the treated cells is once, once a day, two consecutive days per week, three consecutive days per week, four consecutive days per week, 5 consecutive days per week, six consecutive days per week, or alternatively once every day, every other day, every 3 days, every 5 days, every week, every 2 weeks, every month, every 2 months, every 3 months, every 4 months, every 6 months, or every year. In a preferred embodiment, the administration is once per week.

### Diseases and Conditions

The intact and substantially non-viable Gram-negative bacterial cells as disclosed herein are useful for treating or preventing various types of cancers, as well as infectious diseases and conditions.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. The bacterial cells may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal, such as human, dog, cat, cow, sheep, and the like. In one embodiment, the subject is a human.

In some embodiments, the cancer is solid tumor, including metastatic solid tumor and advanced metastatic solid tumor. In some embodiments, the cancer is leukemia or lymphoma. Non-limiting examples cancers include bladder cancer, non-small cell lung cancer, renal cancer, breast cancer, hepatocellular or liver cancer, pancreatic cancer, urethral cancer, colorectal cancer, head and neck cancer, squamous cell cancer, Merkel cell carcinoma, gastrointestinal cancer, stomach cancer, esophageal cancer, ovarian cancer, renal cancer, and small cell lung cancer.

Additional cancerous diseases or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

In some embodiments, for the treatment of cancer, a secondary agent can be used in combination with the treated cells. The combinatory use may be concurrent (such as at the same time, on the same day, or in the same dosage form), or sequential. Example secondary agents include, without limitation, cyclophosphamide, IL-2, a non-steroidal anti-inflammatory drug (NSAID) such as indomethacin, an anti-PD-1 or anti-PD-L1 antibody, and an anti-CD20 antibody (e.g., rituximab). Each of these agents has shown to be able to synergize with the treated cells in treating cancers.

In one example, the cancer is colorectal cancer and the secondary agent is cyclophosphamide and/or an anti-CTLA-4 antibody. In another example, the cancer is pancreatic cancer and the secondary agent is cyclophosphamide, an NSAID (e.g., indomethacin), an anti PD-1 or anti-PD-L1 antibody, or the combinations thereof. In another example, the cancer is liver cancer and the secondary agent is an NSAID (e.g., indomethacin), an anti PD-1 or anti-PD-L1 antibody, or the combination thereof. In another example, the cancer is non-Hodgkin's lymphoma and the secondary agent is cyclophosphamide or an anti-CD20 antibody (e.g., rituximab).

The intact, stabilized and substantially non-viable Gram-negative bacterial cells as disclosed herein are also useful for strengthening the immune system of a subject, and thus are useful for preventing or treating diseases and conditions via improved immune response. The intact and substantially non-viable Gram-negative bacterial cells as disclosed herein can also be used as vaccines or immunologic adjuvants for subjects at risk of developing such diseases or conditions.

In some embodiments, the diseases or conditions to be treated are infectious diseases. In some embodiments, the infection is caused by bacteria, fungi, parasites or viruses. In particular, the presently disclosed bacterial cells can be uniquely suitable for treating viral infections, optionally with a secondary anti-infectious agent.

In some embodiments, the disease being treated is HBV infection. In some embodiments, the disease being treated in HIV infection.

The administration may also start before an actual infection, or before an infection is diagnosed, as a preventative vaccine or prevention.

In some embodiments, the one or more additional therapeutic agent may be inhibitors of cyclooxygenase (COX) enzymes, such as NSAIDS, including 6MNA, aspirin, carprofen, diclofenac, fenoprofen, flufenamate, flubiprofen, ibuprofen, lndomethacin, ketoprofen, ketorolac, meclofenamate, mefenamic acid, naproxen, niflumic acid, piroxicam, sulindac sulphide, suprofen, tenidap, tolmetin, tomoxiprol, zomepirac, celexocib, etodolac, meloxicam, nimesulide, diisopropyl fluorophosphate, L745,337, NS398, rofecoxib, SC58125, S-aminosalicylic acid, ampyrone, diflunisal, nabumetone, paracetamol, resveratrol, salicin, salicylaldehyde, sodium salicylate, sulfasalazine, sulindac, tamoxifen, ticlopidine, and valeryl salicylate.

In some embodiments, the one or more additional therapeutic agent may be agonists of stimulatory immune checkpoints such as CD27, CD28, CD40, CD122, CD137, OX40, GITR and ICOS, or antagonists of inhibitory immune checkpoints such as A2AR, B7-H3, B7-H4, CTLA-4, IDO, KIR, LAG3, PD-1, PD-L1, TIM-3, and VISTA.

Non-limiting examples of the one or more additional therapeutic agent also include abacavir, acyclovir, adefovir, amantadine, amprenavir, ampligen, arbidol, atazanavir, atripla, balavir, cidofovir, combivir, dolutegravir, darunavir, delavirdine, didanosine, docosanol, edoxudine, efavirenz, emtricitabine, enfuvirtide, entecavir, ecoliever, famciclovir, fomivirsen, fosamprenavir, foscarnet, fosfonet, ganciclovir, ibacitabine, imunovir, idoxuridine, imiquimod, indinavir, inosine, integrase inhibitor, interferon type III, interferon type II, interferon type I, interferon, lamivudine, lopinavir, loviride, maraviroc, moroxydine, methisazone, nelfinavir, nevirapine, nexavir, nitazoxanide, nucleoside analogues, norvir, oseltamivir (Tamiflu®), peginterferon alfa-2a, penciclovir, peramivir, pleconaril, podophyllotoxin, protease inhibitor, raltegravir, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine. In one embodiment, the additional therapeutic agent is interferon alpha.

In some embodiments, the second agent is an exogenous antigen. As demonstrated in Example 2, the regression of established murine breast carcinoma tumors by Decoy was considerably enhanced when the test animals were transfected with a foreign antigen (HER2). It is contemplated that Decoy can function as a “super-adjuvant” agent that promotes presentation of antigen molecules. Tumor cells, however, frequently develop the ability to hide their antigen to evade immune reaction. When an exogenous antigen is provided, Decoy can facilitate presentation of the antigen to the immune system, leading to pronounced immune reaction.

Accordingly, another embodiment of the present disclosure provides a method for treating or preventing cancer in a patient in need thereof, which entails administering to the patient (a) an effective amount of a composition comprising intact, stabilized and substantially non-viable E. coli cells which have been treated in such a way as to result in about 70% to 99% reduction of lipopolysaccharide (LPS)-associated endotoxin activity when measured by the Limulus Amebocyte Lysate (LAL) assay as compared to untreated, wild-type E. coli cells, and (b) an exogenous antigen associated with the cancer.

In some embodiments, the antigen is selected from EGFR, Her2, EpCAM, CD20, CD30, CD33, CD47, CD52, CD133, CD73, CEA, gpA33, mesothelin, Mucins, NY-ESO, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP, Tenascin, and Claudin 18.2.

Yet another embodiment of the present disclosure provides a method for treating or preventing an infectious disease in a patient in need thereof, which entails administering to the patient (a) an effective amount of a composition comprising intact, stabilized and substantially non-viable E. coli cells which have been treated in such a way as to result in about 70% to 99% reduction of lipopolysaccharide (LPS)-associated endotoxin activity when measured by the Limulus Amebocyte Lysate (LAL) assay as compared to untreated, wild-type E. coli cells, and (b) an exogenous antigen associated with the infectious disease. In some embodiments, the antigen is a viral or bacterial antigen.

The exogenous antigen can be encapsulated in, coupled to, or expressed by the E. coli cells, in some embodiments. For instance, if the antigen is a protein, a vector encoding the antigen can be introduced to the E. coli cells enabling them to express and secrete the antigen. In some embodiments, the exogenous antigen is separate from the E. coli cells. In some embodiments, they are admixed before administration to achieve concurrent administration. In some embodiments, they are administered separately, either concurrently or sequentially. If administered separately, their administration schedule and frequency can be determined based on needs.

In some embodiments, the second agent is PBMC, or engineered immune cells. Engineered immune cells are being used in clinic, such as immune cells transduced to express recombinant chimeric antigen receptors (CARs) or T-cell receptors (TCR). In some embodiments, the immune cells are T cells, macrophage cells, monocytes, NK cells, or myeloid cells. In some embodiments, the CAR or TCR recognize a tumor associated antigen.

### Formulations, Dosage Forms and Modes of Administration

Formulations and dosage forms of the treated bacterial cells are also provided. In one embodiment, an aqueous formulation is provided that includes the treated cells, along with phosphate buffer, Mg²⁺, and trehalose.

In some embodiments, the phosphate buffer includes or can be prepared with disodium phosphate dihydrate mixed with monopotassium phosphate. In some embodiments, the magnesium ion can be provided with magnesium chloride hexahydrate.

In some embodiments, the trehalose is trehalose dihydrate. In some embodiments, the concentration of trehalose or trehalose dihydrate is 2% to 30% or 5% to 20%, or 8% to 16%, 10% to 14% or 11% to 13% (w/v). In some embodiments, the concentration of trehalose or trehalose dihydrate is 20 mg/mL to 300 mg/mL, 50 mg/mL to 200 mg/mL, 80 mg/mL to 160 mg/mL, 100 mg/mL to 140 mg/mL, or 110 mg/mL to 130 mg/mL.

An example formulation includes 0.5 mg/mL to 2 mg/mL of disodium phosphate dihydrate, 0.1 mg/mL to 0.4 mg/mL of monopotassium phosphate, 3 mg/mL to 12 mg/mL of sodium chloride, 0.05 mg/mL to 0.3 mg/mL of potassium chloride, 0.15 mg/mL to 0.6 mg/mL of magnesium chloride hexahydrate, and 50 mg/mL to 200 mg/mL of trehalose dihydrate, and at a pH of 7.3 to 7.7.

In another embodiment, the formulation includes 0.8 mg/mL to 1.3 mg/mL of disodium phosphate dihydrate, 0.14 mg/mL to 0.25 mg/mL of monopotassium phosphate, 4 mg/mL to 8 mg/mL of sodium chloride, 0.1 mg/mL to 0.2 mg/mL of potassium chloride, 0.2 mg/mL to 0.4 mg/mL of magnesium chloride hexahydrate, and 80 mg/mL to 160 mg/mL of trehalose dihydrate, and at a pH of 7.3 to 7.7.

In some embodiments, the formulation includes 0.1×10⁹/mL to 20×10⁹/mL of the treated cells. In some embodiments, the formulation includes 0.2×10⁹/mL to 10×10⁹/mL of the treated cells. In some embodiments, the formulation includes 0.5×10⁹/mL to 5×10⁹/mL of the treated cells.

In some embodiments, the intact and substantially non-viable Gram-negative bacterial cells in the dosage form have at least about 70% reduction of LPS-associated endotoxin activity (e.g., as measured by the LAL assay) as compared to untreated wild-type bacteria. In some embodiments, the reduction is at least about 70%, 75%, 80%, 85%, 90%, 91%, 92%, 93%, 94%, 95%, 96%, 97%, 98%, 99%, 99.5%, 99.9%, 99.95% or 99.98%. In some embodiments, the reduction is not greater than about 95%, 96%, 97%, 98%, 99%, 99.5%, 99.9%, 99.95%, 99.98% or 99.99%. In some embodiments, the reduction is from about 70% to about 99.99%, from about 80% to about 99.99%, from about 90% to about 99.5% or 99%, from about 91% to about 99%, from about 92% to about 98%, from about 93% to about 97%, from about 94% to about 96%, from about 94.5% to about 95.5%, from about 94% to about 97%, from about 95% to about 98%, from about 96% to about 99%, from about 97% to about 99.5%, or from about 98% to about 99.9%, without limitation.

In some embodiments, the intact and substantially non-viable Gram-negative bacterial cells in the dosage form have at least about 70% reduction of pyrogenicity (e.g., as measured by in vivo rabbit assay) as compared to untreated wild-type bacteria. In some embodiments, the reduction is at least about 70%, 75%, 80%, 85%, 90%, 91%, 92%, 93%, 94%, 95%, 96%, 97%, 98%, 99%, 99.5%, 99.9%, 99.95% or 99.98%. In some embodiments, the reduction is not greater than about 95%, 96%, 97%, 98%, 99%, 99.5%, 99.9%, 99.95%, 99.98% or 99.99%. In some embodiments, the reduction is from about 70% to about 99.99%, from about 80% to about 99.99%, from about 90% to about 99.5% or 99%, from about 91% to about 99%, from about 92% to about 98%, from about 93% to about 97%, from about 94% to about 96%, from about 94.5% to about 95.5%, from about 94% to about 97%, from about 95% to about 98%, from about 96% to about 99%, from about 97% to about 99.5%, or from about 98% to about 99.9%, without limitation.

In some embodiments, the reduction of non-LPS-associated pyrogenicity is at least about 50%, 60%, 70%, 75%, 80%, 85%, 90%, 91%, 92%, 93%, 94%, 95%, 96%, 97%, 98%, 99%, 99.5%, 99.9%, 99.95% or 99.98%. In some embodiments, the reduction is not greater than about 60%, 70%, 75%, 80%, 85%, 90%, 91%, 92%, 93%, 94%, 95%, 96%, 97%, 98%, 99%, 99.5%, 99.9%, 99.95%, 99.98% or 99.99%. In some embodiments, the substantially non-viable bacteria have their viability reduced by at least 80%, 85%, 90%, 95%, 99%, or more.

As demonstrated in the experimental examples, the manufactured treated bacterial cells are surprisingly stable. The stability is reflected with the strong resistance to disruption by sonication and retaining of integrity for long periods during storage. Accordingly, one embodiment of the present disclosure provides a method for providing a therapeutically acceptable composition. In some embodiments, the method entails lyophilizing a plurality of treated bacterial cells as disclosed herein to form a lyophilized composition and storing the lyophilized composition (a) at a temperature of 1° C. to 10° C. for at least 2 months or (b) at a temperature of −15° C. or below for at least 2 years, thereby providing a therapeutically acceptable composition suitable for therapeutic use.

In some embodiments, the storage is at temperature of 1° C. to 10° C. for at least 3, 4, 5, 6, 7, 8, 9, 10, 11 or 12 months, or for at least 1 day, 2, 3, 4, 5, 6, 7, 8, 9, or 10 days, or at least 1 week, 2, 3, 4, 5, 6, 7, 8 or 9 weeks. In some embodiments, the storage is at temperature of −15° C. or below for at least 6, 7, 8, 9, 10, 11 or 12 months, or at least 1, 3, 4, 5, 6, 7, or 8 years.

In some embodiments, the plurality of treated bacterial cells is provided in a solution that further comprises a phosphate buffer, Mg²⁺, and trehalose, such as any of the formulated compositions as disclosed herein. In one embodiment, the formulated composition includes 0.3×10⁹/mL to 5×10⁹/mL of the intact and substantially non-viable E. coli cells, 0.5 mg/mL to 2 mg/mL of disodium phosphate dihydrate, 0.1 mg/mL to 0.4 mg/mL of monopotassium phosphate, 3 mg/mL to 12 mg/mL of sodium chloride, 0.05 mg/mL to 0.3 mg/mL of potassium chloride, 0.15 mg/mL to 0.6 mg/mL of magnesium chloride hexahydrate, and 50 mg/mL to 200 mg/mL of trehalose dihydrate, and at a pH of 7.0 to 7.7.

The compositions are formulated for pharmaceutical administration to a mammal, preferably a human being. Such pharmaceutical compositions of the invention may be administered in a variety of ways, including intravenous, intra-tumoral, subcutaneous, intra-dermal, intra-muscular, intra-vesical, intra-nasal, or peritoneal.

In one embodiment, the treated cells or the composition (prior to or after storage as disclosed herein) is administered parenterally. The term “parenteral” as used herein includes subcutaneous, intravenous, intramuscular, intra-vesical, intra-articular, intra-synovial, intrasternal, intrathecal, intrahepatic, intralesional and intracranial injection or infusion techniques.

Sterile injectable forms of the compositions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compositions may be formulated for parenteral administration by injection such as by bolus injection or continuous infusion.

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intra-vesically, intraperitoneally, parenterally, intramuscularly, or subcutaneously.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Some examples of suitable excipients include lactose, dextrose, sucrose, sorbitol, mannitol, trehalose, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1. Characterization of the Decoy Treated Bacteria Product

This example summarized the results of some non-clinical studies of the “Decoy” product of treated bacteria. Decoy is composed of attenuated then 100% killed, stabilized and intact bacteria produced from non-pathogenic E. coli, producing ˜90% reduction in LPS endotoxin activity and pyrogenicity (“treated bacteria,” also referred to as “Decoy bacteria” or simply “Decoy”). Specific human immune receptor (TLRs and NOD) activation or agonist activity was assessed with a panel of human embryonic kidney reporter cell lines (HEK293), each transfected with a different immune receptor. STING agonist activity was assessed with a human monocytic cell line carrying a luciferase reporter gene responsive to induction of type 1 interferons. Endotoxin activity and pyrogenicity were quantified using Limulus amebocyte lysate and in vivo rabbit assays. Bacterial integrity was assessed by electron and light microscopy. Stability was assessed by sensitivity to disruption by probe sonication. The E. coli were auxotrophs, requiring diaminopimelic acid (DAP) for growth. Mammals do not make DAP, resulting in an inability of the live strains to proliferate in mammals. This provides a fail-safe mechanism, in addition to 100% killing, which enhances safety of the product.

The viability and endotoxin activity of wild-type E. coli before and after various treatments, under a variety of incubation conditions and times, were evaluated using optical density measurements at 600 nm, plating efficiency, and Endosafe Endochrome K Kinetic LAL assay. Treatments included polymyxin B, an antibiotic that lyses Gram-negative bacteria, but is also known to neutralize endotoxin, as well as agents with potential to kill (phenol) or kill and stabilize the bacteria (formaldehyde, glutaraldehyde). Since the antibiotic action of polymyxin B produces cell lysis, but is unrelated to the neutralization of endotoxin activity, the goal of the exploratory studies was to develop a process with associated conditions that could significantly reduce endotoxin activity and kill 100% of the cells, while still keeping the cells intact.

It was determined that incubation of E. coli cells with polymyxin B in the presence of fermentation medium and magnesium chloride at reduced temperature resulted in the desired reduction in endotoxin activity without cell lysis. A subsequent incubation with glutaraldehyde in Phosphate Buffered Saline (PBS) resulted in 100% killing of the cells. The concentrations and exposure times for polymyxin B and glutaraldehyde were determined by dose-response and time-course experiments. Confirmation that the product was intact was carried out with light microscopy (after Gram-staining) and electron microscopy. All bacterial cultures used to produce Decoy-treated products were routinely evaluated for DAP-dependence and percent viability before, during and after the treatment process. Trehalose (final concentration of 12%) was added to the product as a cryoprotectant, prior to freezing and storage at −70° C.

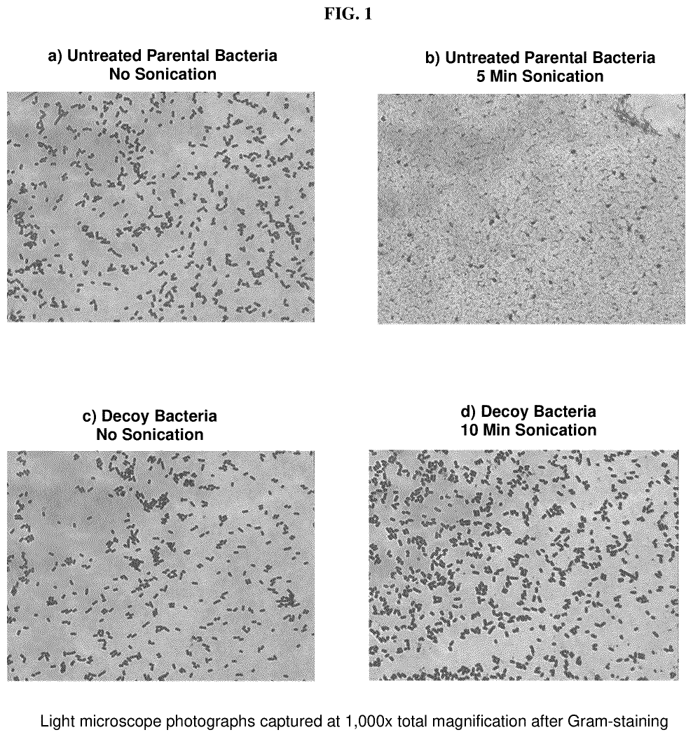

Stabilization of the treated or Decoy bacteria, relative to the untreated parental bacteria, was demonstrated by showing that the untreated parental bacteria could be easily disrupted by sonication. Whereas, the treated or Decoy bacteria were resistant to disruption by sonication, as demonstrated by light microscopy (after Gram-staining) (FIG. 1). Sonication was performed using a Fisher Scientific sonic dismembrator fixed with a microprobe at setting #3 for 5-10 minutes. Sonication was carried out in a round bottom tube with the probe kept just below the surface for efficient sonication of the material.

The stability of the Decoy bacteria was also tested after long-term storage. When frozen and stored at −70° C., the Decoy bacteria were stable for at least 2 years. Under 4° C. storage conditions, the Decoy bacteria were stable for at least 6 months with no detectable degradation.

The pyrogenicity of untreated bacteria (DB100) and Decoy was evaluated in a dose range-finding study using New Zealand White (NZW) rabbits (4 rabbits/group). Untreated bacteria (3×10⁴ bacteria per mL) and Decoy doses (3×10⁵ bacteria per mL and 9×10⁵ bacteria per mL) were administered as a slow i.v. injection via marginal ear vein to NZW rabbits (10 mL per dose). Untreated bacteria increased rectal temperature by 0.7° C. at the dose of 3×10⁴ bacteria per mL, Decoy increased rectal temperatures by 0.1° C. at the dose of 3×10⁵ bacteria per mL and between 0.8° C. and 1.0° C. at the dose of 9×10⁵ bacteria per mL, demonstrating an approximately 30-fold or 97% reduction in pyrogenicity (Table 1).

TABLE 1
Treatment of Live E. coli Cells with Polymyxin B and Glutaraldehyde
(GA) Kills the Bacteria and Significantly Reduces LPS Endotoxin
Activity and In Vivo Pyrogenicity

| | | LPS Endotoxin Activity | Pyrogenicity Threshold |
|---|---|---|---|
| Treatment | Live Bacteria | (LAL Assay) | (Rabbit Assay) |
| No Treatment | 100% | 44.7 Units/10⁶ Bacteria | 3 × 10⁴ Bacteria/mL |
| Decoy | 0% | 3.6 Unit /10⁶ Bacteria | 9 × 10⁵ Bacteria/mL |
| | (Killed all bacteria) | (92% reduction) | 97% reduction |
| | | | (requires more bacteria to |
| | | | increase rabbit temperature) |

LAL = limulus amebocyte lysate;
LPS = lipopolysaccharide.
Acute LD₁₀₀ of Decoy bacteria in mice (3 × 10¹⁰) is 3-fold higher than untreated bacteria (1 × 10¹⁰).

Untreated (DB100) E. coli bacteria and Decoy were administered as single i.v. doses to groups of 3 BALB/c mice in the range from 1×10⁸ to 3×10¹⁰ bacteria per mouse and observed for up to 15 days. A dose of 1×10¹⁰ DB100 was 100% lethal (found dead or moribund), whereas the same dose of Decoy produced no deaths. The 3-fold higher dose of 3×10¹⁰ produced lethality. Clinical observations on mice during the Decoy treatment in this and subsequent pharmacology studies were primarily transient weight loss and transient ruffled fur. Although, antitumor activity was seen with doses of Decoy bacteria that produced no clinical signs (e.g., 3×10⁷ bacteria per animal) in the combination setting.

Specific human immune receptor (TLRs and NOD) activation or agonist activity was assessed with a panel of human embryonic kidney reporter cell lines (HEK293), each transfected with a different immune receptor. Activation of the receptor stimulates NF-kB activity, which induces expression of a reporter gene producing secreted embryonic alkaline phosphatase (SEAP), which can be quantitated. STING agonist activity was assessed with a human monocytic cell line (THP1) carrying a luciferase reporter gene responsive to induction of type 1 interferons. Control cell lines without the transfected immune receptor, but containing the reporter gene system are used to determine specificity of the response. Activity is confirmed if ≥2-fold induction of the reporter gene is produced by the experimental compound. Experimental compound activity is also compared to that of commercially available positive control receptor activators. Treated Decoy bacteria contained agonists or activators of all functional TLR receptors and heterodimers, as well as NOD2 and STING (Table 2).

The pharmacodynamic effect of Decoy on the secretion of cytokines and chemokines was evaluated with human peripheral blood mononuclear cells (PBMCs). TLR ligands such as E. coli LPS, ds E. coli DNA, E. coli peptidoglycan (PGN), Flagellin, R848, Polyinosinic-polycytidylic acid (poly I:C), and CpG oligonucleotide (ODN) were also assessed in these studies. Initial experiments were carried out with incubation for 24, 48 and 72 hours, as well as with and without immune cell activation with anti-CD3. Activation by anti-CD3 was not required for induction of cytokine and chemokine secretion from PBMCs by Decoy bacteria. In addition, maximal induction of secretion of most cytokines and chemokines occurred after 48 hours. Treated Decoy bacteria induced higher levels of most cytokines and chemokines, relative to the same dose of untreated Decoy bacteria (Table 2). Essentially all cytokines and chemokines participate in activation of innate and/or adaptive (including anti-tumor and anti-viral) immune pathways, but can also induce significant toxicity if expressed in the wrong place, at the wrong time, for too long, or at an inappropriately high level.

TABLE 2
Decoy Treatment Does Not Reduce (Most) Cytokine Secretion
by Human PBMCs

| Secretion by | | | Decoy- |
|---|---|---|---|
| Human PBMCs | Untreated | Decoy-Treated | Treated Bacteria |
| In Vitro | Bacteria | Batch #1 | Batch #2 |
| Cytokine | 48 hr pg/mL peak (mean of triplicates) | | |
| | at same bacterial dose for each cytokine | | |
| GM-CSF | 1,094 | 1,197 | 1,695 |
| IFNγ | 175,866 | 47,488 | 55,321 |
| IL-1β | 11,976 | 17,651 | 19,232 |
| IL-6 | 78,422 | 98,534 | 89,332 |
| IL-8 | 126,942 | 166,769 | 145,921 |
| IL-10 | 6,970 | 7,620 | 5,882 |
| IL-12p70 | 176 | 528 | 428 |
| TNFα | 49,782 | 77,919 | 99,247 |

GM-CSF = granulocyte-macrophage colony-stimulating factor;
IL = interleukin;
PBMCs = peripheral blood mononuclear cells;
TNF = tumor necrosis factor.

In addition to the outer membrane Toll-Like Receptor 4 (TLR4) agonist endotoxin or LPS, Gram-negative bacteria, such as E. coli, contain agonists for a variety of other TLRs, including TLR2, TLR2/1, TLR2/6, TLR5, and TLR9. Evidence has also been published demonstrating the presence of TLR3, TLR7, and TLR8 agonist RNA in E. coli. E. coli also contain agonists of the NOD-Like Receptors (NLR) NOD1 and NOD2, as well as agonists of the stimulator of interferon (IFN) genes (STING). Since Decoy manufacturing modifies the surface, but keeps the bacteria intact, it is likely that various other TLR agonists and immune activating danger signals are retained in the product. This was verified by screening Decoy bacteria against a panel of human embryonic kidney (HEK) cell lines, each individually transfected with a single, different human or mouse immune receptor or receptor heterodimer, including TLR2, 2/1, 2/6, 3, 4, 5, 7, 8, 9, NOD1, NOD2 or STING, each coupled to a reporter gene read-out. Decoy bacteria demonstrated agonist activity against or activation of all human TLRs, most mouse TLRs, as well as NOD2 and STING (Table 3).

TABLE 3
Activation of Human Immune Receptors by Decoy Bacteria
or Positive Controls

| | Target-Expressing | | Target-Expressing | |
|---|---|---|---|---|
| | Cell Line | | Cell Line | |
| | Fold Induction of | | Fold Induction of | |
| | Reporter Signal | | Reporter Signal | |
| | Decoy | Decoy | Positive Control | Positive |
| | 2 × 10⁷/mL | 5 × 10⁸/mL | 0.04× | Control 1× |
| TLR2/1 | 21.6 | 24.0 | 1.9 | 19.7 |
| TLR2/6 | 3.2 | 13.0 | 16.3 | 23.0 |
| TLR2 | 38.1 | 39.1 | 3.8 | 34.5 |
| TLR3 | 1.4 | 7.5 | 1.2 | 19.3 |
| TLR4 | 19.3 | 17.2 | 17.0 | 18.5 |
| TLR5 | 1.4 | 8.1 | 4.3 | 30.2 |
| TLR7 | 2.1 | 6.2 | 8.5 | 10.9 |
| TLR8 | 2.3 | 13.1 | 20.6 | 22.5 |
| TLR9 | 2.6 | 13.3 | 6.7 | 13.5 |
| NOD2 | 2.7 | 9.0 | 18.6 | 18.9 |
| STING | 9.7 | 9.1 | 34.4 | 107.4 |

Significant evidence has accumulated demonstrating that TLR/TLRa signaling can enhance or is required for innate and adaptive anti-tumor immune responses. Several single, pure, or synthetic TLR agonists have been shown to have anti-tumor activity in preclinical studies and have been tested in clinical cancer trials. This example compared induction of cytokine and chemokine secretion from PBMCs by Decoy to induction by various single TLR agonists. Table 4 demonstrates that Decoy bacteria induced higher levels of cytokines and chemokines than any of four individual TLR agonists, including purified E. coli LPS. These results demonstrate that Decoy bacteria are better activators of immune cell cytokine and chemokine secretion than many single, pure TLR agonists and support the finding that Decoy bacteria contain multiple TLR agonists and other immune stimulators, in addition to LPS.

TABLE 4
Decoy Bacteria Induce Higher Levels and Unique Pattern of
Cytokine Secretion by Human PBMCs Relative to Pure (Single)
TLR Agonist Therapeutics (Study AB64785)

| | CpG ODN | Poly(I:C) | R848 | LPS | Decoy |
|---|---|---|---|---|---|
| | (TLR9) | (TLR3) | (TLR8) | (TLR4) | (TLR2, 4, 5, 9) |
| Anti-Tumor | | | | | |
| Cytokine | 48 hr pg/mL (full titration peak/mean of triplicates) | | | | |
| GM-CSF | 0 | 0 | 87 | 175 | 1,695 |
| IFNγ | 7 | 103 | 31,324 | 29,416 | 75,530 |
| IL-1β | 1 | 22 | 9,990 | 4,631 | 19,232 |
| IL-6 | 241 | 129 | 40,555 | 54,174 | 89,332 |
| IL-8 | 2,436 | 1,452 | 116,135 | 143,459 | 145,921 |
| IL-10 | 374 | 8 | 940 | 3,542 | 5,882 |
| IL-12p70 | 4 | 18 | 253 | 109 | 428 |
| TNFα | 51 | 208 | 33,393 | 24,944 | 99,247 |

GM-CSF = granulocyte-macrophage colony-stimulating factor;
IFN = interferon;
IL = interleukin;
LPS = lipopolysaccharide;
PBMCs = peripheral blood mononuclear cells;
TLR = toll-like receptor;
TNF = tumor necrosis factor.

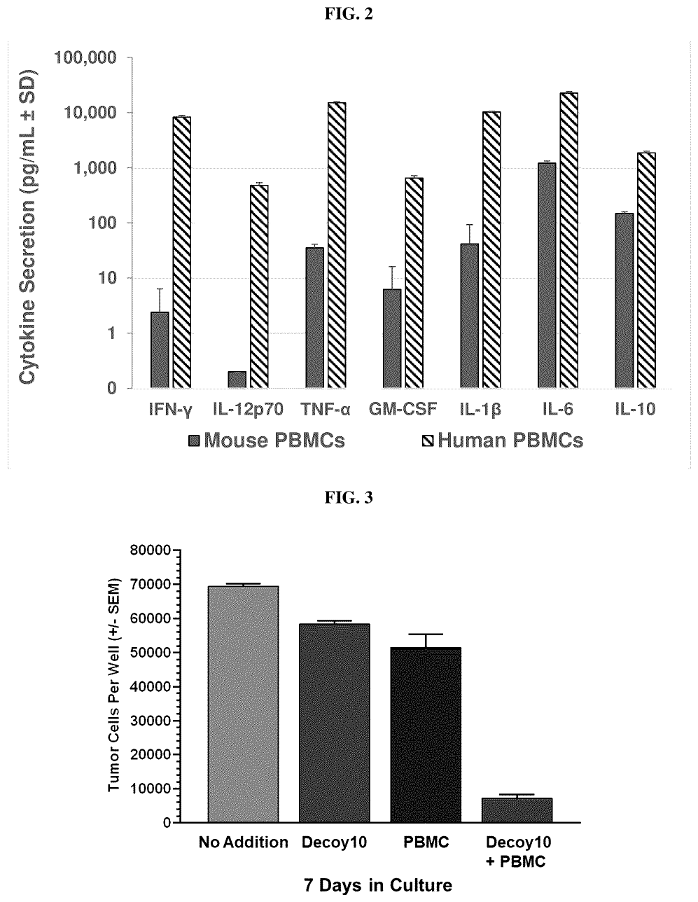

Humans are approximately 500 to 1,000-times more sensitive to i.v. purified LPS--mediated toxicity than mice on a ng/kg basis, and this difference in sensitivity has been found to be correlated with differences in ability of LPS to induce cytokine secretion from human and mouse immune cells. Furthermore, it was found that the difference in sensitivity is mediated by factors in blood or serum. Additional in vitro pharmacology studies were carried out and shows that equivalent numbers of Decoy bacteria induced 10 to 3,500-times more cytokine secretion from equivalent numbers of human PBMCs (in human serum), relative to mouse PBMCs (in mouse serum) (FIG. 2).

Since cytokines mediate both anti-tumor activity and toxicity, this finding that human immune cells are more sensitive than mouse immune cells to induction of cytokines (including anti-tumor cytokines) by Decoy bacteria suggests that if there is an acceptable anti-tumor therapeutic index in mice, there may also be one in humans.

### Example 2. Non-Clinical Studies of Decoy

This example summarized the results of some non-clinical studies of the Decoy product of treated bacteria.

### In Vitro Activity in Human Breast Carcinoma Cells

The growth inhibitory effect and cytotoxicity of Decoy was evaluated in human PBMCs and in an MDA-MB-231 breast tumor cell line co-culture model. PBMCs (1×10⁴ cells/well, 1×10⁵ cells/well, and 1×10⁶ cells/well) alone or in combination with MDA-MB-231 Nuclight red stable cells (2×10⁴) were seeded in 96-well plates and treated with Decoy (3×10⁶ bacteria) for 96 and 168 h. At concentrations of 1×10⁵ cells/well and 1×10⁶ cells/well, PBMCs alone inhibited the growth of MDA-MB-231 tumor cells, but Decoy alone showed minimal inhibition of tumor cell growth. The combination of Decoy and a non-inhibitory concentration of PMBCs (1×10⁴ cells/well) produced an apparent synergistic effect of 90% growth inhibition and cytotoxicity of tumor cells after 7-day incubation (FIG. 3).

### In Vivo Activity in a Mouse Colorectal Carcinoma Model

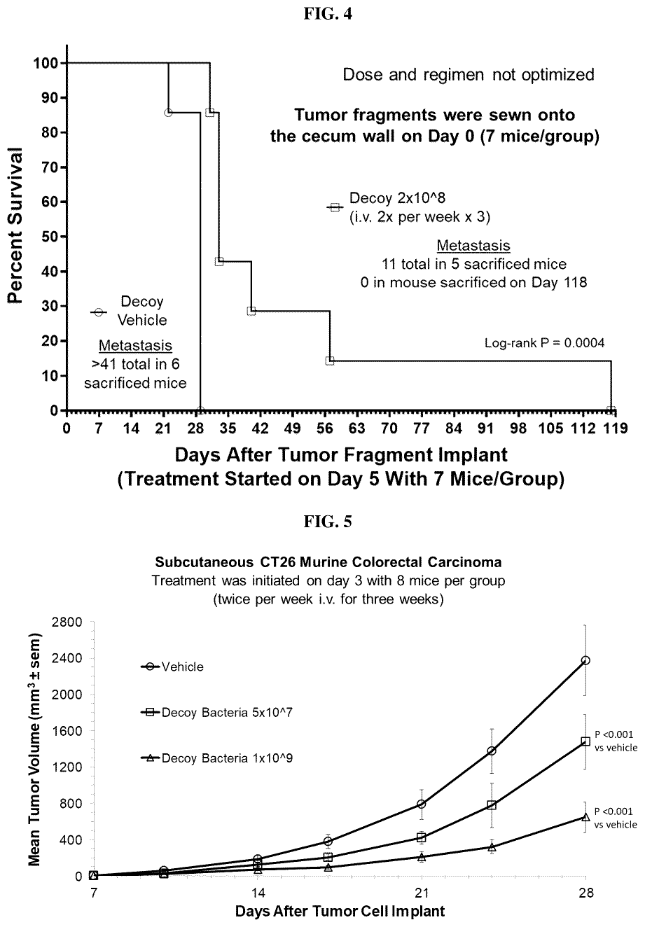

The antitumor activity of Decoy alone was studied in an orthotopic model of human colorectal carcinoma using female BALB/c mice (7 mice per group) bearing green fluorescent protein-labeled CT26 tumor fragments derived from s.c. tumors. At 5 days post-implantation on the cecum (surgery), mice were treated i.v. with Decoy vehicle or with Decoy (2×10⁸ bacteria//animal i.v. twice weekly [QD×2] for 3 weeks). Results are shown in FIG. 4. Tumor growth was measured by palpation in-life and tumor weight post-mortem. Metastasis was measured by green fluorescent protein imaging with “open” animals, post-mortem. In this experiment, Decoy alone was shown to prolong survival and inhibit metastasis. Maximum transient weight loss during the first week of Decoy treatment was 1.3% relative to the start of treatment, with no weight loss in subsequent weeks of treatment.

The antitumor efficacy of Decoy was evaluated in female BALB/c mice (8 animals per group) using the s.c. mouse syngeneic CT26.WT colorectal carcinoma model. Exploratory tests were also carried out with cyclophosphamide administered p.o. in drinking water and an anti-CTLA-4 antibody, alone and in combination with Decoy. Single agent Decoy was administered i.v. at 5×10⁷ or 1×10⁹ bacteria per mouse twice per week (Q4D) for 3 weeks starting on Day 3 after tumor cell implant. Both doses produced statistically significant tumor growth inhibition compared to vehicle control as determined by an unpaired T test on Days 24 and 28 (low dose) and Days 21, 24, and 28 (high dose) (FIG. 5). The high dose produced statistically significant inhibition compared to vehicle control as measured by time to reach 1,000 mm³ (report). No body weight loss was recorded for the low Decoy dose and only one measurement of transient body weight loss (0.56%) was recorded for the high Decoy dose.

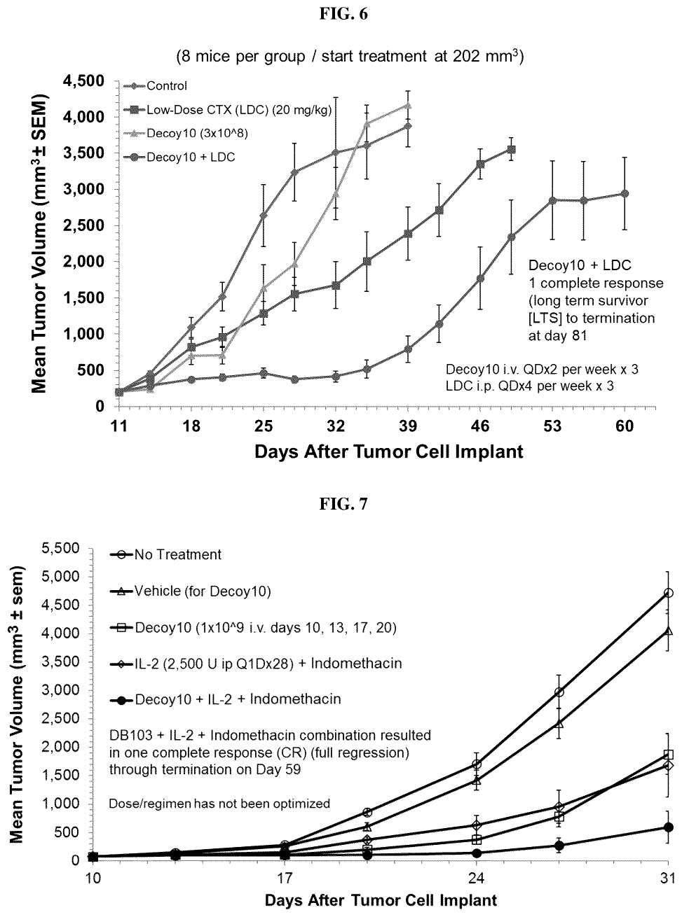

The antitumor activity of Decoy in combination with low dose intraperitoneal (i.p.) cyclophosphamide (LDC) was studied in a s.c. model of colorectal carcinoma using female BALB/c mice (8 mice per group) bearing established CT26 tumors (same as CT26.WT). At 11 days post implantation, when the average tumor size was 202 mm³, mice received no treatment, Decoy alone (1.5, 3.0, or 6.0×10⁸ bacteria/animal i.v. twice weekly [QD×2] for 3 weeks), LDC (20 mg/kg i.p. QD×4 starting one day before the Decoy treatments for 3 weeks), or a combination of 3.0×10⁸ Decoy and LDC and these results are shown in FIG. 6.

In this study, Decoy alone did not produce a statistically significant anti-tumor effect at 1.5 or 3.0×10⁸ bacteria per mouse, but did produce a statistically significant tumor growth delay at 6.0×10⁸ bacteria per mouse of 25% increased lifespan (% ILS). LDC alone produced a 57% ILS, which was statistically significant. However, the combination of 3×10⁸ Decoy and LDC produced a statistically significant ILS of 89% and also resulted in one full, durable tumor regression, with long-term survival to termination at Day 81. Transient maximum weekly weight loss during weekly QD×2 Decoy treatment at doses of 1.5, 3.0, and 6.0×10⁸ was 8.9% to 11.2% in the first week, 2.2% to 5.4% in the second week and 0% to 1.9% in the third week, as expected for LPS tolerance, which has been reported with i.v. LPS administration in mammals, including humans. Decoy +LDC treatment produced transient weight loss in the range of 12-16% for each for the three weeks of treatment, without loss of any animals.

The antitumor efficacy of Decoy as a single agent or in combination with i.p. IL-2+p.o. indomethacin was evaluated in female BALB/c mice (5 animals per group) using the syngeneic s.c. mouse CT26.WT colorectal carcinoma model. CT26.WT cells were injected s.c. in the right flank of BALB/c mice. Starting on Day 10, when the average tumor size was 75 mm³, groups of mice received no treatment or were administered Decoy vehicle, Decoy (1×10⁹ bacteria/mouse, i.v. Q4D×2×2 weeks; IL-2 (2,500 U/animal i.p. QD×28)+indomethacin (14 μg/mL, p.o., ad libitum, QD×28) or Decoy+IL-2+indomethacin. Single agent Decoy and IL-2+indomethacin each produced a statistically significant inhibition of tumor growth (median tumor growth delay of 7.0 and 7.1 days, respectively). The combination of Decoy +IL-2+indomethacin produced a statistically significant median tumor growth delay of 18.9 days and also resulted in one tumor regression or complete response through termination on Day 60 (FIG. 7). Maximum transient body weight loss with Decoy alone was 5% in the first week and 0% in the second week of treatment. Maximum transient body weight loss with Decoy +IL-2+indomethacin was 7% in the first week and 3% in the second week of treatment.

Several additional compounds were tested in this study, including anti-GITR antibody, INF-gamma (accidently used at 10× intended dose, which produced toxicity), and phenformin, individually and in combination with each other and/or Decoy. Some of the groups described above received these compounds after Day 31. Significant additional anti-tumor activity and/or synergism was not seen with these agents or combinations.

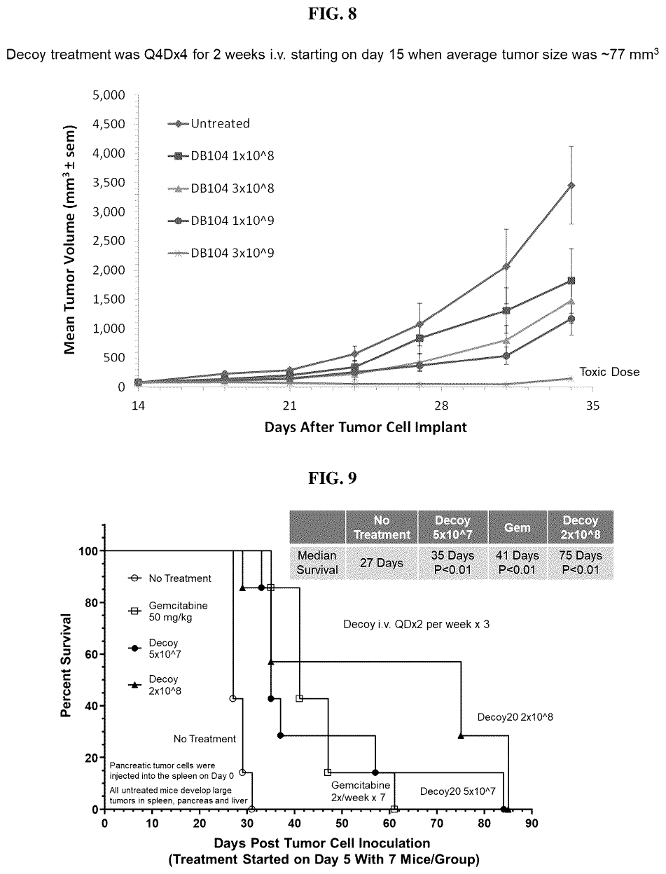

In an in vivo pharmacodynamic study, the anticancer activity of Decoy (DB104) as single agent or in combination with cyclophosphamide (CTX) was evaluated in female BALB/c mice (5 animals per group) using the s.c. mouse CT26.WT colorectal carcinoma model. Mouse CT26.WT colorectal carcinoma cells were implanted (injected) into the right flank of BALB/c mice. Animals were randomized and treatments were initiated (Starting Day=SD) with a mean group tumor weight of 75 to 79 mg. Mice were administered Decoy (3×10⁸ bacteria/animal, i.v. Q4D×4 [SD-F1]; Q8D×4 [SD-F1]), Decoy (1×10⁸, 3×10⁸, 1×10⁹, 3×10⁹ bacteria/animal, i.v., Q4D×4 [SD+1]), and CTX (50 mg/kg, i.p., Q4D×2 [SD]; Q8D×2 [SD]). A group of animals that received no treatment was used as the control. Decoy at a dose of 3×10⁹ bacteria/animal was not tolerated resulting in 4 mortalities, but all other treatments were tolerated. Transient body weight loss in the range of 5-9% was seen for 1-2 days after Decoy administration for all groups except the highest (toxic) Decoy dose, with less body weight loss after repeat dosing, probably due to LPS tolerance. Both schedules of CTX produced significant tumor growth inhibition without producing any tumor regressions. Combination of Decoy with Q4D×2 CTX produced significant tumor growth inhibition and one full tumor regression or complete response through termination at Day 91 suggesting a potential synergistic effect. Treatment with Decoy as a single agent at all doses (FIG. 8), and combined with the corresponding CTX treatment, were effective in the inhibition of the growth of s.c. implanted CT26.WT colorectal carcinoma tumors compared with untreated mice (p<0.05). Only Decoy and CTX combined treatments on the Q4D schedules were more effective against CT26.WT colorectal carcinoma growth than CTX or Decoy treatment alone.

### In Vivo Activity in a Mouse Metastatic Pancreatic Carcinoma Model

In a primary pharmacodynamic study, the in vivo efficacy of Decoy, gemcitabine, low-dose cyclophosphamide (LDC) and Decoy+LDC was evaluated in female C57BL/6 mice (7 animals per group) using the intrasplenic Pan02 pancreatic carcinoma mouse model. Pancreatic tumor cells were surgically implanted (injected) into the spleen of C57BL/6 female mice on Day 0. Mice were administered Decoy (5×10⁷ and 2×10⁸ bacteria/animal i.v. QD×2 per week for 3 weeks), gemcitabine (50 mg/kg i.p. BIW×7), or LDC (20 mg/kg i.p. QD×4×3 weeks). A group of untreated animals was used as the control. All untreated animals developed large tumors in the spleen, liver and pancreas, requiring termination around Day 30. Decoy produced a significant increase in median survival time at doses of 5×10⁷ and 2×10⁸ per mouse. Maximum transient weight loss (relative to start of treatment) in the higher/lower dose Decoy groups was 9.8%/8.2% in the first week of treatment, 2.6%/2.1% in the second week of treatment and no weight loss during the third week of treatment. The positive control drug, gemcitabine, produced a significant increase in median survival in between the results obtained with the two Decoy doses. Combination of Decoy with LDC did not enhance survival beyond that obtained with Decoy alone in this model (FIG. 9).

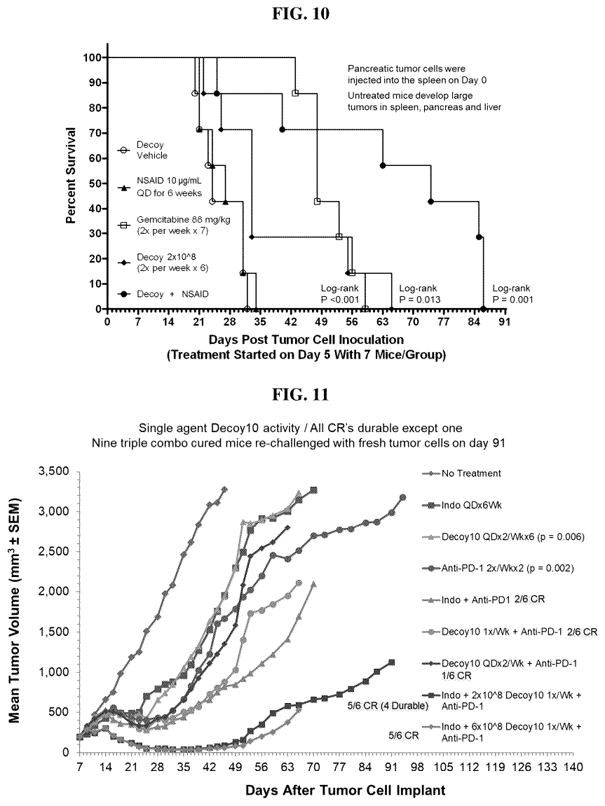

In a second experiment with the same intrasplenic Pan02 pancreatic carcinoma mouse model, the therapeutic effects of Decoy, gemcitabine, and indomethacin, as well as Decoy+gemcitabine and Decoy+indomethacin were evaluated. Mice were administered Decoy (2×10⁸ bacteria/animal i.v. QD×2 per week×3 weeks), gemcitabine (several different doses and schedules), and low-dose oral indomethacin (10 μg/mL p.o. QD ad libitum×31 days). A group of animals administered Decoy vehicle was used as the control. Single agent Decoy and single agent gemcitabine increased survival. Gemcitabine did not appear to produce additive or synergistic activity with Decoy. Single agent indomethacin did not increase survival significantly, but appeared to enhance survival in a potentially synergistic fashion in combination with Decoy (38% ILS increase with Decoy alone extended to 208%). In this combination setting, maximum transient weight loss was 6.4% during the first week of treatment, 1.5% during the second week of treatment and there was no weight loss relative to the start of treatment in all subsequent weeks of treatment (FIG. 10).

### In Vivo Activity in a Subcutaneous, Established, Mouse Hepatocellular Carcinoma Model

The therapeutic efficacy of Decoy alone and in combination with anti-PD-1 or indomethacin and anti-PD-1 was evaluated in female BALB/c mice (6 animal per group) using a s.c. H22 mouse syngeneic hepatocellular carcinoma model. Treatment was initiated when the average tumor size was 194 mm³. Mice were administered single agent Decoy at 2×10⁸ bacteria/animal, i.v., slow push QD×2 per week×6 weeks. Decoy (2×10⁸ bacteria/animal QD×2 and 1× per week for 6 weeks) was tested in combination with anti-PD-1, as well as at various dose levels up to 6×10⁸ bacteria/animal 1× per week for 6 weeks in combination with indomethacin and anti-PD-1. Indomethacin was administered at 10 μg/mL, p.o., ad libitum daily in drinking water×6 weeks (alone and in combinations) and anti-PD-1 was administered at 10 mg/kg, i.p., BIW×2 weeks (alone and in combinations). A group of animals that received no treatment was used as the control in the study. No Decoy-related mortality was observed at any dose of Decoy or combination. Transient body weight loss was observed after each Decoy treatment. The maximum weekly average group body weight loss for QD×2 Decoy over 6 weeks of treatment was −6.91%, −5.34%, −5.48%, −3.28%, −0.94%, and 0%, demonstrating the well-established tolerance phenomenon seen with i.v. LPS administration in mammals, including humans. Combination of Decoy with indomethacin and antiPD-1 produced a high percentage of durable tumor regressions, but did not lead to a significant increase in body weight loss or other signs of toxicity. Administration of 2×10⁸ bacteria/animal with indomethacin and anti-PD-1 for 6 weeks produced weekly average group body weight loss of −6.37%, −3.81%, −3.86%, −0.58%, −1.49%, and 0%. Administration of 6×10⁸ bacteria/animal with indomethacin and anti-PD1 for 6 weeks produced weekly average group body weight loss of −8.30%, −4.99%, −5.35%, −2.94%, −3.70%, and −0.17%.

Single agent Decoy administered QD×2 weekly for 6 weeks produced a small, but statistically significant increase in life-span (27.5% ILS). Similar results were seen with single agent indomethacin or anti-PD-1 (30% ILS and 37.5% ILS, respectively), without any tumor regressions. All combination treatments produced a statistically significant increase in ILS. In addition, combination of 1× per week Decoy with antiPD-1 led to 2/6 durable full tumor regressions, and 1/6 durable full tumor regressions with QD×2 per week Decoy. Treatment with indomethacin+Decoy+anti-PD-1 led to 5/6 tumor regressions in each of the groups receiving 2×10⁸ or 6×10⁸ Decoy. All but 1 of the 10 regressions were durable until the termination of the experiment at Day 143 (FIG. 11).

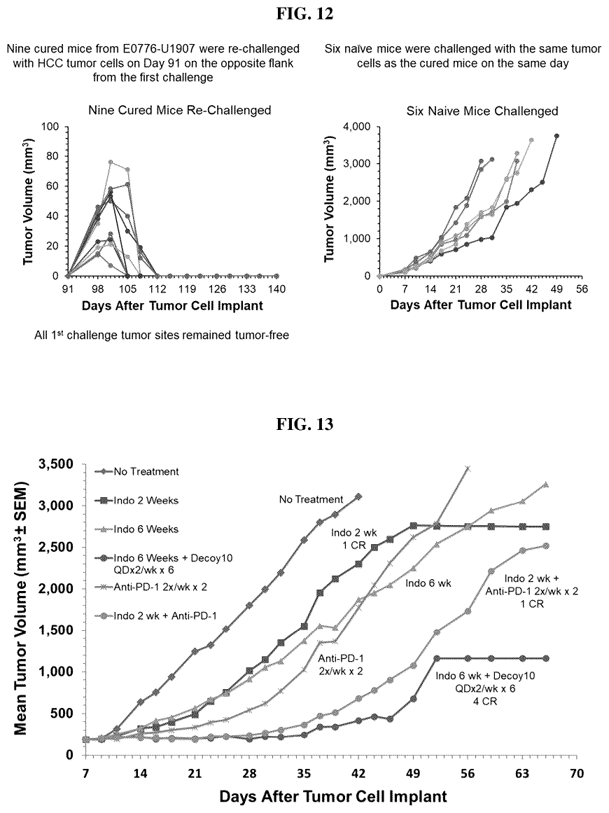

The nine cured animals were re-challenged s.c. with fresh tumor cells on Day 91 on the opposite side of the back from the first tumor challenge (no further drug treatment) and both tumor sites on each animal were followed for an additional 52 days. Tumors started to grow at all of the re-challenge sites, but then spontaneously and fully regressed, demonstrating 100% immunological memory. Naïve animals that received the same tumor cells on the same re-challenge day produced progressively growing tumors that eventually exceeded the 3,000 mm³ human sacrifice limit (FIG. 12).

The therapeutic efficacy of indomethacin, anti-PD-1, Decoy+indomethacin, and Decoy+indomethacin+anti-PD-1 was assessed in a second study with female BALB/c mice (6 animals per group) using the s.c. H22 hepatocellular carcinoma mouse syngeneic model (Study E0776-U1802). Decoy was tested in the combination settings QD×2 per week, 2× per week, and lx per week. Decoy was not tested as a single agent in this study. Decoy was tested at 2×10⁸ bacteria/animal, i.v. slow push for 6 weeks. Indomethacin was tested at 10 μg/mL in drinking water, p.o. ad libitum daily for either 2 or 6 weeks. Anti-PD-1 was tested at 10 mg/kg, i.p. 2× per week for 2 weeks. Dosing was started when the average tumor size was 184 mm³. A group of untreated animals was used as the control. One mortality was observed after three weeks of dosing with Decoy (QD×2 per week)+indomethacin+anti-PD-1. The same combination, as well as all other treatments and combinations, was well-tolerated by all other mice. The maximum transient weight loss for all groups receiving Decoy was 7.5% to 10.1%, occurred in the first week of treatment and was not higher in the triple combination, compared to the double combinations. Transient weight loss after Decoy dosing generally decreased in subsequent weeks of treatment and was less than 1% after the last treatment in Week 6 for all triple combination groups, demonstrating tolerance.

Indomethacin (2 weeks and 6 weeks) and anti-PD-1 produced a statistically significant increase in lifespan (ILS), with one regression after treatment with indomethacin for two weeks. Indomethacin (2 weeks)+anti-PD-1 also produced an increased ILS and 1 regression. Decoy (QD×2 for 6 weeks)+indomethacin (6 weeks) produced an increased ILS and 4 regressions. Since we have never seen regressions with Decoy alone in this model and did not see any regressions with 6week indomethacin treatment, the results strongly support a synergistic interaction. All regressions were durable through the termination of the experiment on Day 91 (FIG. 13).

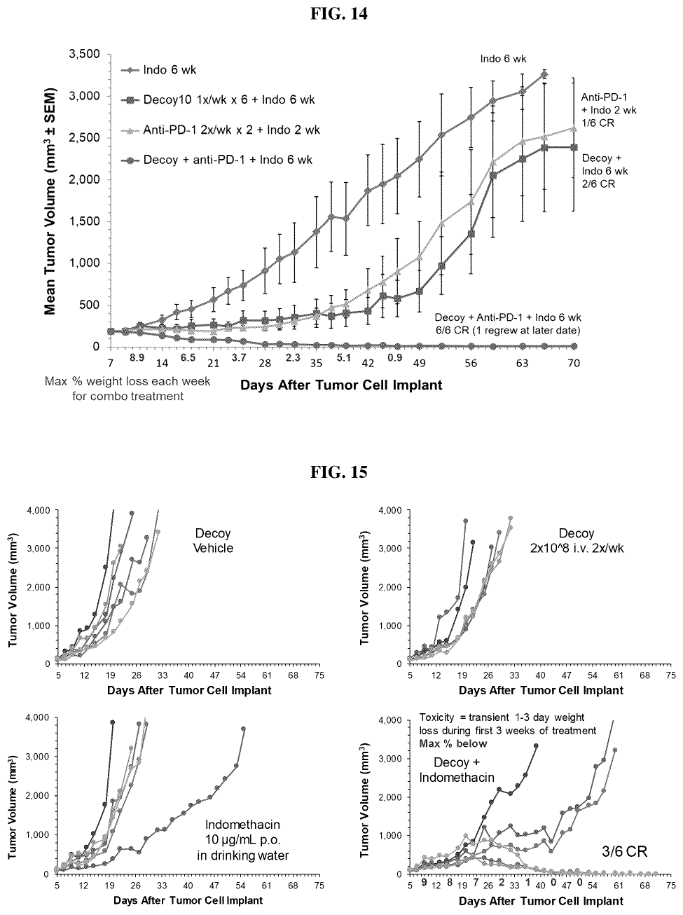

In the same study, 2× per week and 1× per week Decoy was tested in combination with indomethacin, producing 1/6 and 2/6 durable regressions, respectively. Decoy was also tested QD×2 per week, 2× per week, and 1× per week in combination with indomethacin (6 weeks)+anti-PD-1, producing 4, 5, and 6 regressions or partial regressions respectively. The best results were obtained with 1× per week Decoy+anti-PD-1+6 weeks indomethacin, which produced 6 full regressions, 5 of which were durable to termination at Day 91 (FIG. 14).

The therapeutic efficacy of Decoy, low-dose cyclophosphamide (LDC), indomethacin, gemcitabine (GEM), 5-FU and various combinations were tested in an additional study with female BALB/c mice (6 animals per group) using the s.c. H22 hepatocellular carcinoma mouse model. Decoy was tested at 2×10⁸ bacteria/animal, i.v. slow push for 4 weeks (single agent) or 7 weeks (combination with indomethacin). Indomethacin was tested at 10 μg/mL in drinking water, p.o. ad libitum daily for either 4 or 7 weeks. A group of animals treated with Decoy vehicle was used as control. Treatment was started when the average tumor size was 123 mm³. There were no treatment-related mortalities, except with GEM, which produced weight loss of 20% as a single agent and deaths in combination with Decoy. GEM and LDC produced weak, but statistically significant, single agent anti-tumor activity. Single agent anti-tumor activity was not seen with Decoy in this study. Combination activity was seen with LDC+indomethacin (no regressions) and with Decoy+indomethacin, resulting in 3/6 full regressions (complete responses—CR), which were durable until study termination on Day 71. Transient weight loss was not seen with indomethacin administration. Single agent Decoy produced transient weight loss of 8.2% in the first week of treatment, 5.3% in the second week of treatment and no weight loss relative to the start of treatment in the 3^{rd} and 4^{th} weeks of treatment, demonstrating tolerance. Combination with indomethacin produced slightly more weight loss in the first 3 weeks of treatment, with little or no weight loss in Weeks 4-7 of treatment (FIG. 15).

Since Decoy+indomethacin was found to synergize with anti-PD-1 to produce high percentage regressions, the therapeutic efficacy and Decoy therapeutic index of Decoy+indomethacin in combination with anti-PD-1 was evaluated in female BALB/c (6 animals per group) using the s.c. H22 mouse hepatocellular carcinoma model. Treatment was initiated when the average tumor size was 205 mm³. Mice were administered Decoy 1× per week at 3×10⁷, 1×10⁸, 3×10⁸, or 1×10⁹ bacteria/animal i.v. slow push for 6 weeks+indomethacin 10 μg/mL in drinking water p.o. ad libitum daily for 6 weeks+anti-PD-1 2× per week at 10 mg/kg i.p. for 2 weeks. A group of animals that received no treatment was used as the control in the study. A Decoy dose-response was assessed with Decoy treatment prior to anti-PD-1 treatment each week and with the first weekly anti-PD-1 treatment prior to Decoy treatment each week. Decoy and anti-PD-1 were also assessed as single agents against non-established tumors, but not against the established tumors in this study. In other studies, the 3 agents produced minimal or no single agent activity and no regressions as a single agent in this model. Interestingly, single agent anti-PD-1 lead to eventual regression of 2/6 tumors when administered to mice one day after tumor inoculation (non-established tumors), highlighting the well-established activity of antiPD-1 against non--established tumors, but not relatively large, established tumors (>100 mm³) at initiation of treatment.

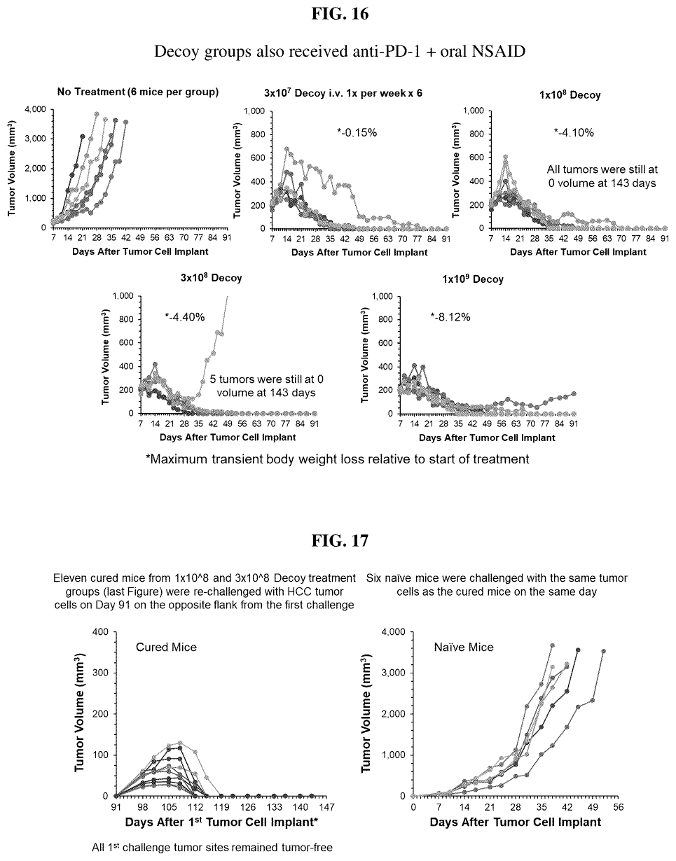

The 3-way combination (initiated with 205 mm³ tumors), regardless of order of treatment with Decoy or anti-PD-1, produced 4 to 6 durable regressions per group of 6 (5-6/6 with anti-PD-1 first and 4-6/6 with Decoy first). Both regimens produced 6/6 regressions at the lowest dose of Decoy (3×10⁷ per animal) with no or minimal transient weight loss (−0.15%, −3.23%) in the first week and less or no weight loss in subsequent weeks of treatment. The 3-way combination with Decoy at 1×10⁸ per animal produced 6/6 or 5/6 regressions (two regimens) with a maximum transient weight loss of −4.10% or −5.55%, decreasing or 0% in subsequent weeks of treatment. Combination with 3×10⁸ Decoy per animal produced 5/6 regressions (both regimens) with a maximum transient weight loss of −4.40% or −5.35%, decreasing or 0 in subsequent weeks of treatment. Combination with 1×10⁹ Decoy per animal produced 5/6 or 4/6 regressions (both regimens) with a maximum transient weight loss of −8.12% or −8.08%, decreasing or 0 in subsequent weeks of treatment. The consistent reduction in weight loss observed upon multiple treatment demonstrates the tolerance phenomenon with Decoy. There were no treatment-related deaths, demonstrating a Decoy therapeutic index in the combination setting of at least 33-fold. In addition, besides transient weight loss, there were no other clinical signs of toxicity of any kind noted in this study. The results for the regimen where the mice received anti-PD-1 first are shown in FIG. 16.

Furthermore, 11 mice with regressed tumors at Day 91 (1×10⁸ and 3×10⁸ Decoy Groups) exhibited 100% immunological memory, as re-challenge of the mice with the same (fresh, viable) tumor cells on the opposite flank, relative to the first tumor challenge (with no further treatment), resulted in initiation of tumor growth, followed by complete rejection (FIG. 17). The initial tumor regressions were true tumor eradications, as evidenced by following all of the mice for 91 days after tumor implantation, and following the original tumor sites on rechallenged mice for a total of 143 days.

The s.c. H22 HCC model was used to evaluate in vivo plasma cytokine and chemokine induction by p.o. indomethacin (10 μg/mL in drinking water QD starting Day-0), i.v. Decoy (2×10⁸ bacteria/animal once per week starting Day-1), i.p. anti-PD-1 (10 mg/kg twice per week starting Day-0) and the various combinations found to induce tumor regression. Female BALB/c mice with ˜200 mm³ H22 tumors were randomized on Day-0 into 8 groups, each containing 3 sub-groups. All of the possible treatment approaches or combinations, including no treatment, were carried out. Mice from each of two subgroups from each group (5 mice per sub-group) were sacrificed 6 and 24 hours after no treatment, single, 2-way, or 3-way combination treatment. In the case of 2way and 3-way combination treatment, mice were sacrificed 6 and 24 hours after the first treatment instance of the second or third component of the combination. Plasma was prepared from each mouse and 32-plex ELISA-based cytokine/chemokine analysis was carried out. A third sub-group from each group (6 mice each) was treated for one week, with tumor volumes measured at randomization, once during the week and at the end of one week of treatment. Mice were sacrificed, then tumors were harvested and RNA was isolated. The 48 RNA samples were analyzed for expression of 770 genes involved in immune response pathways, using NanoString technology.

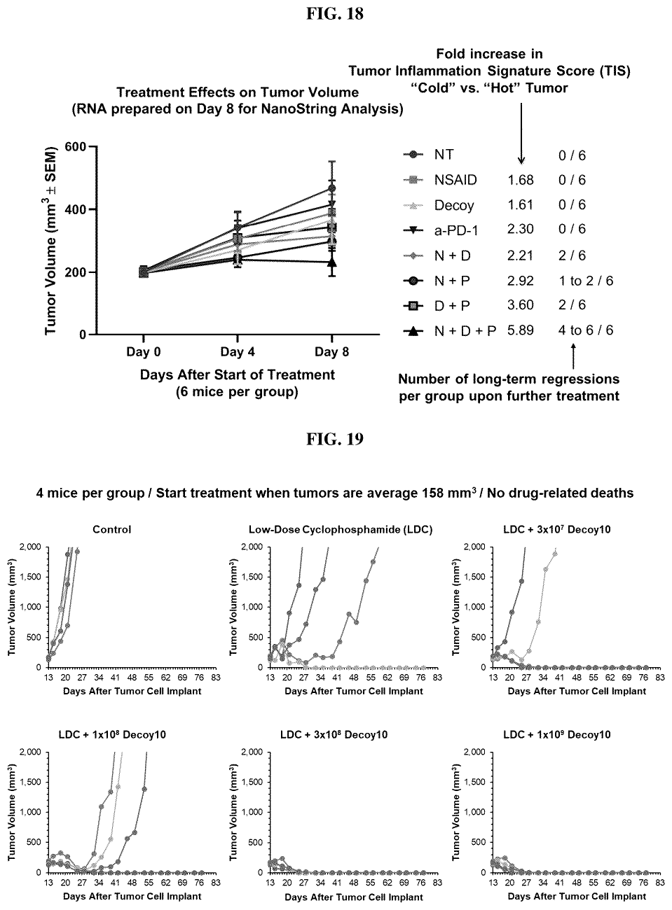

Indomethacin induced expression of only 2/32 cytokines/chemokines, and only at 6 hours after initiation of treatment. Decoy induced expression of 9/32 cytokines/chemokines and, as with indomethacin, levels were only significant compared to no treatment at 6 hours. Surprisingly, no cytokine/chemokine induction was observed 6 or 24 hours after initiation of antiPD-1 treatment. In the combination settings, under conditions where tumor growth inhibition or initiation of regression was observed, statistically significant induction of cytokine/chemokine expression was observed with 18 to 28 out of 32 cytokines/chemokines, including at both 6 and 24 hours for many of the cytokines/chemokines. Transient body weight loss relative to the day of randomization (4 days after randomization) was −1.63% for the no treatment group, −0.65% for indomethacin, −8.63% for Decoy, and −1.74% for anti-PD-1. There was no increase in weight loss in any of the combination groups, relative to single agent treatment. Tumor growth inhibition after one week of treatment, relative to no treatment was 17% for indomethacin, 21% for Decoy, 11% for anti-PD-1, 33% for indomethacin+Decoy, 36% for indomethacin+anti-PD-1, 26% for Decoy+anti-PD-1 and 50% for indomethacin+Decoy+anti-PD-1. At termination, after only one week of treatment (Day 8), 5 out of 6 tumors in the 3-way combination group were smaller in size, compared to the measurements on Day 4 (FIG. 18). Other than transient weight loss, no other clinical signs of toxicity were observed with any mice in any group. It is notable, and surprising, that significant increases in cytokine and chemokine expression (in plasma) were observed in the combination groups without any increase in body weight loss or any other clinical signs of toxicity.

Of the 32 cytokines/chemokines assayed, most (23/32) have been shown to be able to stimulate or contribute to anti-tumor activity in pre-clinical models and some clinical settings. Many of the cytokines/chemokines have also been shown to limit or inhibit anti-tumor activity and/or contribute to toxicity. The result with any particular cytokine/chemokine or combination is highly dependent on many variables, including the animal, model, concentration, location, and timing of cytokine/chemokine expression. It is notable, and surprising, that in the H22 HCC anti-tumor efficacy settings, this example observed high percentage tumor eradication with a therapeutic index of at least 33-fold, decreasing weight loss with no other signs of toxicity upon multiple weeks of treatment, and no apparent increase in clinical signs of toxicity when Decoy bacteria administration was combined with anti-PD-1 therapy (plus or minus indomethacin), despite an apparent synergistic induction of cytokine/chemokine expression, relative to single agent treatment.

Mice with 200 mm³ s.c. HCC tumors (6 mice per group) were not treated or treated as described for efficacy studies and the cytokine analysis with indomethacin (NSAID), Decoy and/or anti-PD-1 for one week, followed by tumor extraction, RNA isolation and analysis of the expression of 770 immune pathway-related and control genes by NanoString gene expression technology. NanoString analysis included evaluation of the well-established Tumor Inflammation Signature (TIS), which provides an indication of the anti-tumor immunological environment in the tumor, with a lower score indicating low activation (“cold tumor”) and a higher score indicating higher activation or potential for an anti-tumor immune response (“hot tumor”). FIG. 18 also demonstrates that the treatments increase the TIS signature, and this is roughly correlated with the progression from single to double to triple combination, which is also correlated with the observed ultimate anti-tumor activity seen in studies where treatment was extended multiple weeks.

Additional NanoString gene expression analysis was carried out evaluating a wide variety of anti-tumor-associated immune system genes, cells, and pathways. The results were validated based on RNA quality and analysis of housekeeping gene expression, with the heatmap results being representative of Log 2based changes in gene expression relative to mean values across the analysis.

Single agent treatment resulted in broad increases in innate and adaptive immune gene/cell/pathway expression in 1 or 2 tumors/mice per group of 6, possibly associated with some tumor growth inhibition. Double agent treatment increased the number of tumors/mice per group exhibiting broad innate and adaptive immune gene/cell/pathway activation, possibly associated with increased tumor growth inhibition and some tumor regressions. The triple agent combination was associated with broad innate and adaptive immune gene/cell/pathway activation in essentially all tumors/mice, highly consistent with the high percentage regression and tumor eradication seen in this setting. Extremely similar results were obtained for general immune gene/cell/pathway activation, cytokine immune genes/pathway activation, chemokine immune genes/pathway activation, innate immune genes/pathway activation and adaptive immune genes/pathway activation.

### In Vivo Activity in a Mouse Model of Non-Hodgkin's Lymphoma

As with the HCC model, Decoy bacteria exhibited low activity as a single agent against the syngeneic murine A20 model of NHL. However, Decoy bacteria were found to synergize with low-dose cyclophosphamide (LDC) to eradicate established tumors in female BALB/C mice. The in vivo therapeutic efficacy of various doses and regimens of Decoy in combination with LDC was evaluated in the s.c. A20 BALB/c Lymphoma Syngeneic model. Decoy (3×10⁷, 1×10⁸, 3×10⁸ and 1×10⁹ bacteria/animal, i.v. slow injection) was administered 2× per week, QD×2, QD×3 or QD4 per week×2 weeks in combination with 20 mg/kg LDC administered i.p. QD×4 per week for two weeks starting 1 day before Decoy to BALB/c mice (4 mice per group). A group of untreated animals was used as the control. LDC alone was also tested. Treatment was initiated on Day 13 when the average tumor size was 158 mm³.

There was one tumor regression with LDC alone, although this was not seen in most studies with LDC alone. The number of partial and full regressions per group of 4 with each regimen and at each Decoy dose (low to high) was 2× per week (1, 2, 1, 2), QD×2 per week (2, 1, 4, 4) (FIG. 19), QD×3 (4, 3, 3, 3) and QD×4 per week (1, 0, 3, 3), with no deaths (related or unrelated to treatment), although some first week and all second week dosing was suspended/missed with QD×4 per week Decoy dosing at the highest two doses, due to excessive transient weight loss (≥20%). QD×2 Decoy per week was considered the optimal regimen, due to the observation of 4/4 full regressions at the two highest doses. The combination of Decoy bacteria with LDC produced higher transient weight loss than with the indomethacin/anti-PD-1 combinations. Transient weight loss reached 17% during the first week and 14% to 17% during the second week of treatment. Decoy tolerance was not generally seen with the Decoy+LDC combination, perhaps because treatment was only carried out for two weeks. Besides transient weight loss, the only other clinical sign of toxicity was ruffled fur. Surprisingly, the combination consistently did not reduce the maximally tolerated dose of Decoy bacteria. Mice routinely tolerated the combination with up to 1×10⁹ Decoy bacteria (QD×2 for two weeks), with no loss of animals.

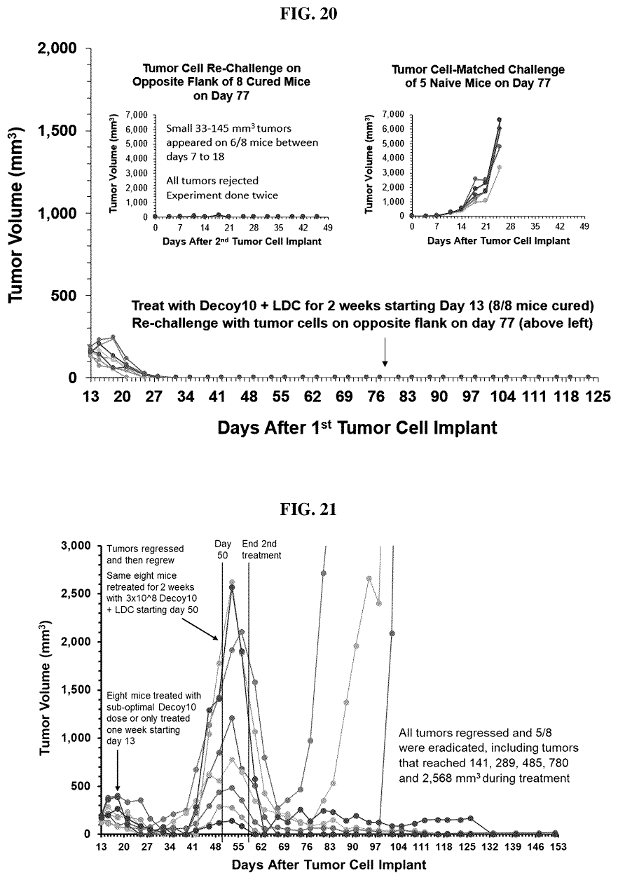

The eight mice with fully regressed tumors from the two highest dose groups in FIG. 19 were rechallenged with fresh tumor cells, on the opposite side of the back from the first tumor challenge, on Day 77 (no additional treatment). All of the new tumor challenges were rejected and the 1^{st} challenge tumor sites remained tumor-free through termination of this part of the experiment on Day 123. Tumors grew normally in non-age-matched naïve mice that received the same fresh tumor cells on the same day as the rechallenge (FIG. 20). Age-matched naïve mice were used for most other tumor re-challenge experiments.

In the same experiment, mice with tumors that initially regressed, but started to regrow due to having received only one week of treatment or a suboptimal dose of Decoy for 2 weeks were administered a two-week round of optimal dose and regimen Decoy (3×10⁸ bacteria/animal, i.v. slow injection QD×2×2 weeks)+LDC, starting at ˜100 to 2,000 mm³ in volume. All of the tumors regressed and 5/8 mice exhibited durable regressions (FIG. 21). These results demonstrate that sensitivity to Decoy technology is not readily lost after initial treatment and that Decoy technology is able to completely regress very large tumors.

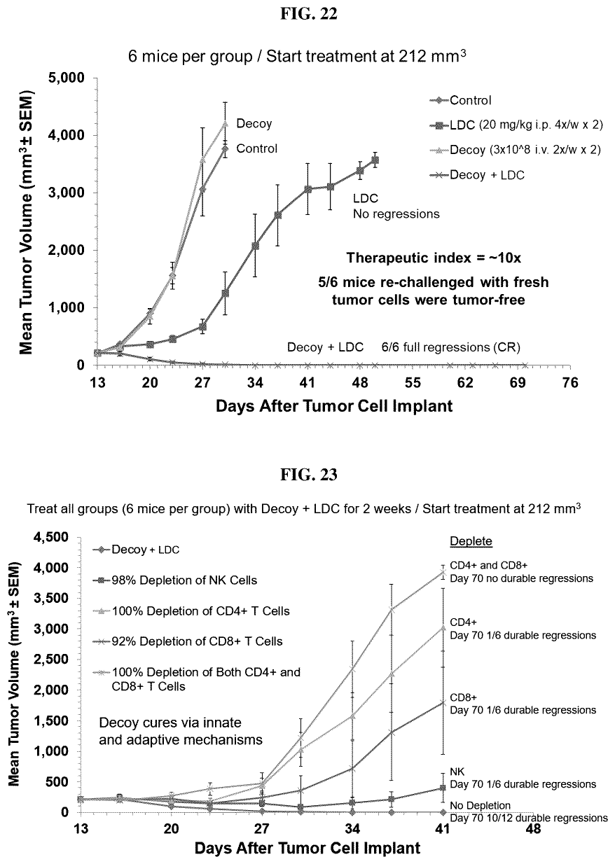

In another experiment, LDC was administered, as above, i.p. at 20 mg/kg four times per week for two weeks 1 day before, during and 1 day after Decoy was administered i.v. slow push QD×2 per week×2 weeks. A group of untreated animals was used as the control. Treatment was started when the average tumor size was 212 mm³. Decoy was tested as a single agent and did not produce anti-tumor activity by itself. As in the previous experiment, tumor eradication was induced by Decoy+LDC (6/6 regressions) and only required Decoy+LDC administration for two weeks (FIG. 22). LDC alone only delayed tumor growth without producing any regressions in this study. Addition of indomethacin with Decoy+LDC was not required and produced 4/6 regressions. Single agent Decoy administered QD×2 for 2 weeks produced maximum transient weight loss of 9.3% in the first week and 3.7% in the second week. Combination with LDC produced 12% transient weight loss in the first week and 13.6% transient weight loss in the second week. However, surprisingly, despite the increased weight loss, the combination consistently did not reduce the maximally tolerated dose of Decoy bacteria. Mice routinely tolerated the combination with up to 1×10⁹ Decoy bacteria (QD×2 for two weeks), with no loss of animals.

In order to investigate the anti-tumor mechanism of action of Decoy treatment, mice were pre-depleted of either natural killer (NK) cells, CD4+ T, CD8+ T or both CD4+ and CD8+ T cells using commercially available reagents. Tumor implantation was carried out during the depletion regimen. Mice were then treated with Decoy (3×10⁸ bacteria/animal i.v. QD×2 per week for two weeks) plus LDC (20 mg/kg i.p. QD×4 per week for two weeks). Extra mice in each group were sacrificed at the time of initiation of treatment in order to verify immune cell depletion. FIG. 23 demonstrates that depletion of any of the immune cell types significantly decreased the anti-tumor activity of the Decoy regimen, reduced the number of CRs/regressions to 1/6 when single immune cells were depleted and abolished all regressions when both CD4+ and CD8+ T cells were depleted. The positive control included 2 groups of 6 mice each (one group received indomethacin), with 10/12 CRs/regressions (6/6 regressions without indomethacin and 4/6 regressions with added indomethacin). The data demonstrate that both the innate and adaptive immune system participate in tumor eradication by Decoy technology, as might be expected with a product that contains multiple TLR agonists and other immune system danger signals. For example, TLR4, which is activated by LPS, has been shown to be important, if not essential, for antigen presentation, processing, and dendritic cell activation. Thus, in addition to immune activation associated with innate and adaptive immune cells, LPS provides a key bridging activity between the innate and adaptive immune systems.

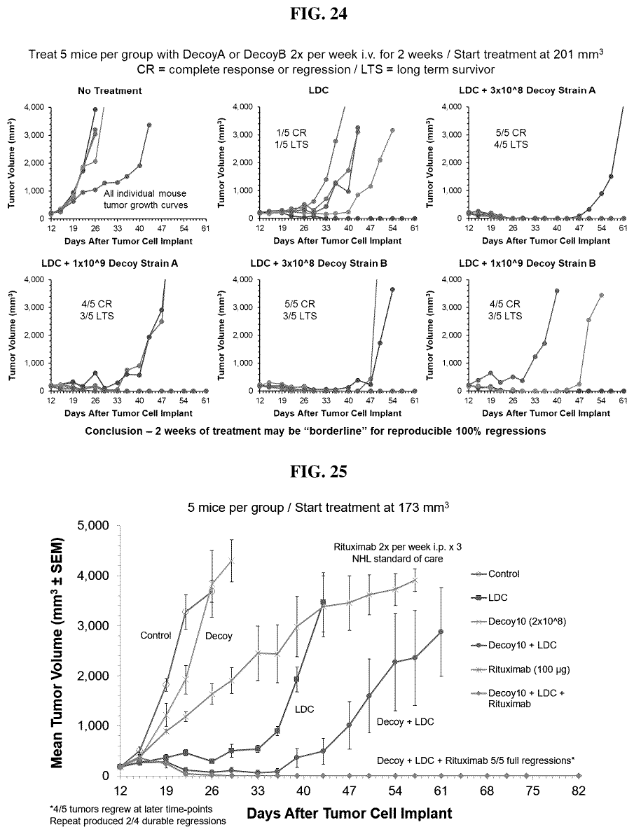

Regression of established A20 mouse syngeneic NHL tumors by Decoy was repeated with two different Decoy products representing two different strains of E. coli. Treatment was started when the average tumor size was 201 mm³. Decoy batch #1 (3×10⁸ and 1×10⁹ bacteria/animal i.v.) or batch #2 (3×10⁸ and 1×10⁹ bacteria/animal i.v.) QD×2 for 2 weeks in combination with LDC (20 mg/kg/animal i.p. QD×4 for 2 weeks) resulted in 4-5/5 regressions and 3-4/5 long-survivors per group (FIG. 24). In this study Decoy was not tolerated as single agents at 1×10⁹ bacteria per animal QD×2 per week for two weeks, with all animals found dead after the third dose. Surprisingly, combination with LDC allowed for successful QD×2 administration of Decoy at 1×10⁹ bacteria per animal, except for the 4^{th} dose for one animal in each Decoy group, due to a weight loss-required dosing holiday. Per above studies, significant transient weight loss was observed when Decoy was administered twice per week on consecutive days, and particularly in combination with LDC. Maximum transient single agent weight loss for the 3×10⁸ Decoy dose over two weeks of treatment was 9.8% and 7.0%, and 10.7% and 7.4% for single agent Decoy at 3×10⁸ bacteria per dose. Combination group maximum transient weight loss for Decoy was 9.7% and 17.6%, and 11% and 16.1% for Decoy. Maximum, transient, weight loss in the 1×10⁹ Decoy single agent dose groups was 12.9% and 15.9% (one week of treatment), and 16.3% to 20.5% in the combination groups over two weeks of treatment.

If Decoy bacteria in combination with LDC are able to activate or enhance innate antitumor immune responses (FIG. 23), then it was contemplated that the bacteria might synergize with innate immune system-mediated therapies in a human tumor xenograft (innate only) setting. This was tested with the human Ramos NHL xenograft model in SCID mice, which lack B and T cells.

The anti-tumor activity of Decoy (2×10⁸ bacteria/animal) as a single agent and in combination with LDC and rituximab was evaluated in a s.c. human Ramos mouse xenograft model of NHL using CB17/SCID mice. Mice were administered Decoy (2×10⁸ bacteria/animal, i.v. slow injection QD×2 per week for 3 weeks, indomethacin (14 μg/mL, p.o. ad libitum QD×21), LDC (20 mg/kg, i.p. QD×4 per week for 3 weeks) and/or rituximab (100 μg/mouse, i.p. BIW for 3 weeks). Treatment was started when the average tumor size was 173 mm³. Indomethacin at 14 μg/mL in drinking water did not produce single agent activity, did not significantly enhance the anti-tumor activity of any of the other treatments, and was found to produce some toxicity with prolonged treatment. All other study compounds, including Decoy, produced statistically significant single agent and combination activity. Maximum transient weight loss for all non-indomethacin groups was generally in the 5% to 10% range. Growth of established Ramos tumors in this model was inhibited or delayed by the standard of care agent rituximab, but regressions or cures were not seen. The combination of Decoy and LDC regressed established Ramos tumors, but was unable to cure mice. Combining Decoy, LDC and rituximab produced relatively durable regressions in 5/5 mice, which lasted through Day 85 (FIG. 25). Four out of five of the fully regressed tumors from FIG. 25 grew back after Day 85. However, cure of at least some percentage of the mice is possible with the 3-way combination as a repeat produced 2/4 durable regressions in mice.

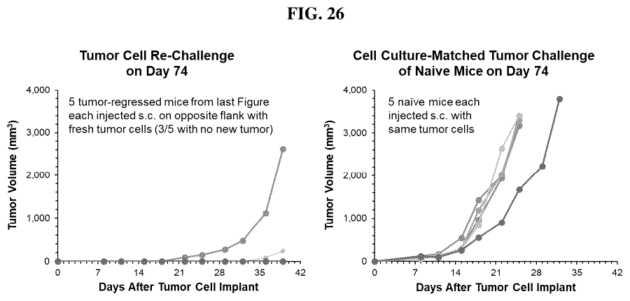

The 5 tumor-regressed mice were re-challenged with fresh Ramos tumor cells (opposite flank from first challenge) on Day 74. Only 2/5 tumors grew out, compared to 5/5 with naïve mice challenged with the same fresh tumor cells (FIG. 26). Thus, this example has demonstrated immunological memory in the innate only setting. This has been reported previously with a small number of other agents, and is considered to be very difficult to achieve. Tumor regression with immunological memory via the innate immune system alone is very rare in preclinical models, but consistent with a multiple TLR agonist mechanism. These results suggest that Decoy technology may synergize with other marketed antibody-dependent cellular cytotoxicity (ADCC or ADCP) mechanism-based, targeted antibody therapeutics, that work, to a large extent, via activation of the innate immune system.

Regression of Established Murine Breast Carcinoma Tumors by Single Agent Decoy after Transfection with a Foreign Antigen

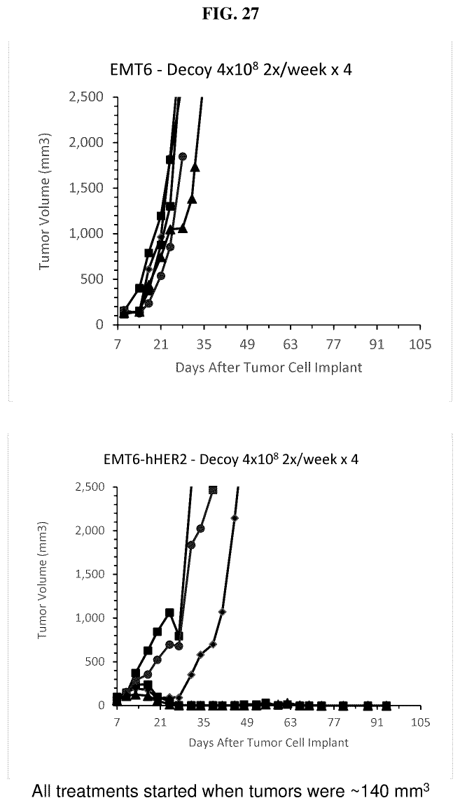

Female BALB/c mice with ˜170 mm³ subcutaneous EMT6 murine breast carcinoma tumors were treated i.v. with 2×10⁸ Decoy bacteria twice per week for 4 weeks. Tumor growth was similar to untreated tumors. Treatment of mice with ˜170 mm³ EMT6 tumors expressing human HER2 receptor led to tumor growth inhibition and complete regression of 2 of 5 established tumors (FIG. 27), demonstrating that, at least in this setting or model, the only aspect of anti-tumor innate/adaptive immunity that Decoy is unable to induce or provide for some mice is the antigen. Thus, provision of an antigen can enhance or synergize with Decoy therapy.

### In Vivo Activity in a Mouse Model of AAV-HBV Chronic HBV Infection

The anti-hepatitis B virus (HBV) efficacy of Decoy was evaluated in two studies. A standard pre-clinical model was used in which the human HBV genome is inserted in an adeno-associated virus (AAV). This hybrid construct is able to infect mouse hepatocytes, producing a chronic HBV-like infection with many of the hallmarks of human HBV infection, including high levels of HBV replication in liver and blood and production of HBe and HBs antigen (HBeAg and HBsAg). AAV-HBV virus is administered to mice and then blood titers of HBV DNA are monitored. Significant and relatively stable blood levels of HBV appear within 28-31 days, at which point treatment is initiated. Indomethacin was administered with Decoy in many groups in the first study, but not in the second study. The inclusion of indomethacin was found not to be required for the anti-viral activity of Decoy.

In the first study, Decoy (two different doses) was tested in combination with indomethacin and also in combination with the clinical standard of care, Entecavir (ETV). ETV and indomethacin were also tested as single agents (first study). In the second study Decoy was tested as a single agent in order to determine if indomethacin was needed for activity. In addition, ETV and mIFN-alpha were tested as single agents and in combination with Decoy (one dose). In the first study, mice were administered Decoy (0.6×10⁸ and 2×10⁸ bacteria/animal, i.v. QD×2 per week×5 weeks), ETV (0.1 mg/kg, p.o. QD per week×5 weeks), and indomethacin (10 μg/mL, p.o. ad libitum×5 weeks). Treatments were started 28 days (indomethacin and ETV) or 29 days (Decoy) after infection. A Decoy vehicle control of PBS (no Ca²⁺ or Mg²⁺) with 2 mM MgCl₂ was used as the control in the study. The best results were obtained with the higher dose of Decoy (presented in the Figures below), although some activity was also observed with the lower dose.

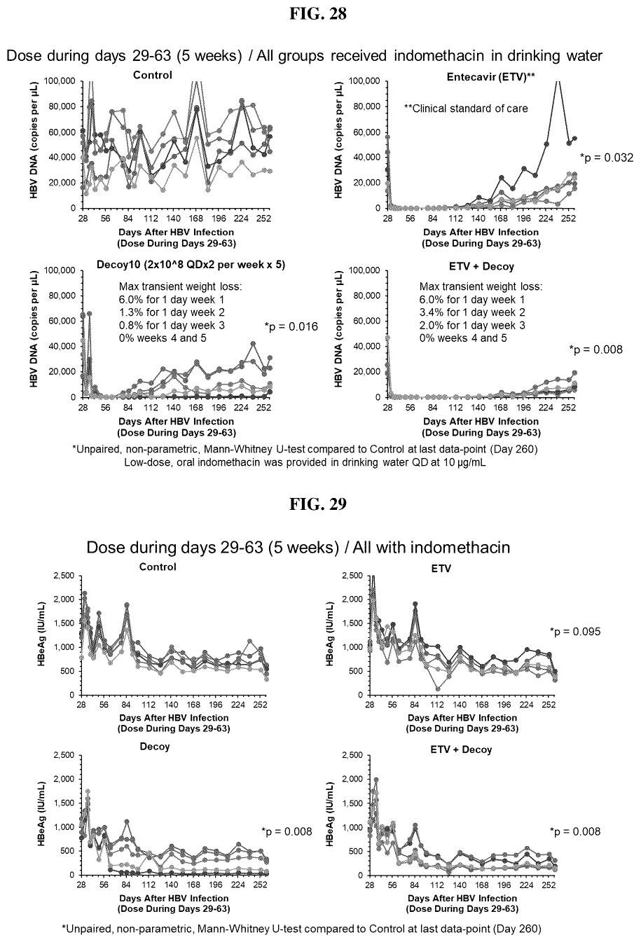

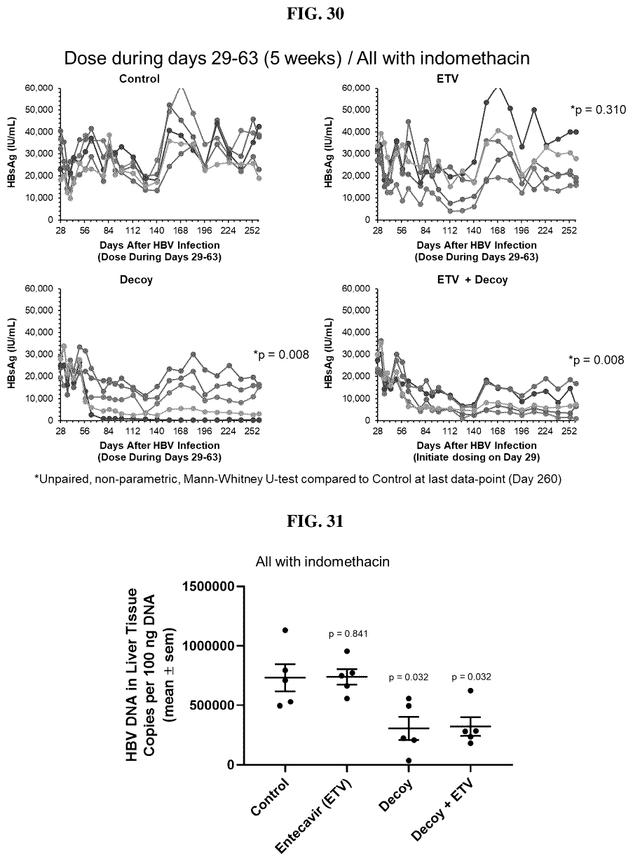

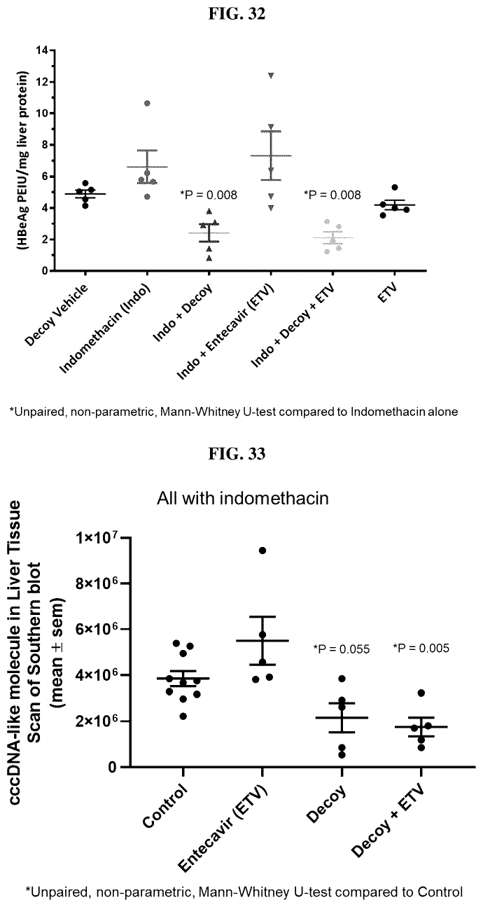

In the first study, single agent indomethacin did not inhibit HBV replication. Decoy bacteria (+indomethacin) and ETV (±indomethacin) significantly inhibited HBV replication (measured in blood) during dosing and for up to 28 weeks after the end of dosing. The combination of Decoy (with indomethacin)+ETV also inhibited HBV replication, perhaps to a greater extent than either Decoy (with indomethacin) or ETV (with indomethacin) alone (FIG. 28). ETV is known not to reduce or inhibit production of HBeAg or HBsAg and this was confirmed in the present studies. Decoy (+indomethacin) treatment led to a significant reduction of both HBeAg and HBsAg in blood during treatment and for up to 28 weeks after the end of dosing (FIG. 29 and FIG. 30). Decoy (+indomethacin) and Decoy+ETV (+indomethacin), but not ETV (±indomethacin), led to a significant reduction of HBV in the liver (FIG. 31). Similar results were observed for HBeAg expression in the liver, where inhibition was only observed with Decoy treatment (+indomethacin) (FIG. 32). Although the precise relationship to HBV covalently closed circular cccDNA in the human infection setting is not known, Decoy (+indomethacin), but not ETV, also reduced the level of a ccc-DNA-like molecule in the AAV-HBV model (FIG. 33). Some or slight inhibition of HBcAg by Decoy (+indomethacin), but not ETV, was also observed in the liver. Decoy-induced body weight loss was mild, transient (6% for 12 days in first week of treatment) and tapered off after the first week of treatment (tolerance phenomenon) (FIG. 28). Three mice in the Decoy (+indomethacin) group and two mice the Decoy+ETV (+indomethacin) group exhibited transient elevated plasma alanine aminotransferase (ALT) levels on 13 occasions during Days 28-56, which all resolved after Day 56 through termination on Day 260. At termination, H&E liver histopathology revealed no Decoy treatment-related changes.

### In Vivo Activity in a Mouse Model of AAV Chronic HBV Infection

In the second in vivo secondary pharmacodynamic AAV-HBV study, the anti-HBV efficacy of Decoy as a single agent (no indomethacin) and in combination with ETV was evaluated in male C57BL/6 mice (5 animals per group) using the standard AAV-HBV mouse model. Mice were administered Decoy (2×10⁸ bacteria/animal, i.v. QD×2 per week×5 weeks), ETV (0.005 mg/kg, p.o. QD×5 weeks) or mIFN-alpha (1000 U/g s.c., TIW×5 weeks). The combinations of ETV (starting 31 days after infection at Day 0 or Day 7)+Decoy and Decoy+mIFN-alpha were also tested. ETV and mIFN-alpha treatment was started 31 days after infection (Day 0) and Decoy was started 32 days after infection (Day 1). A group of untreated animals was included in the study. All treatment was well tolerated in AAV-HB V-infected mice. A mild, transient body weight loss (7% over 2 days in the first week of treatment and then tapering off with subsequent treatment) was noted in Decoy treated mice. One mortality in the single agent Decoy group resulted inexplicably after 4 weeks of treatment (after body weight loss) and was replaced by a back-up mouse (very rare event).

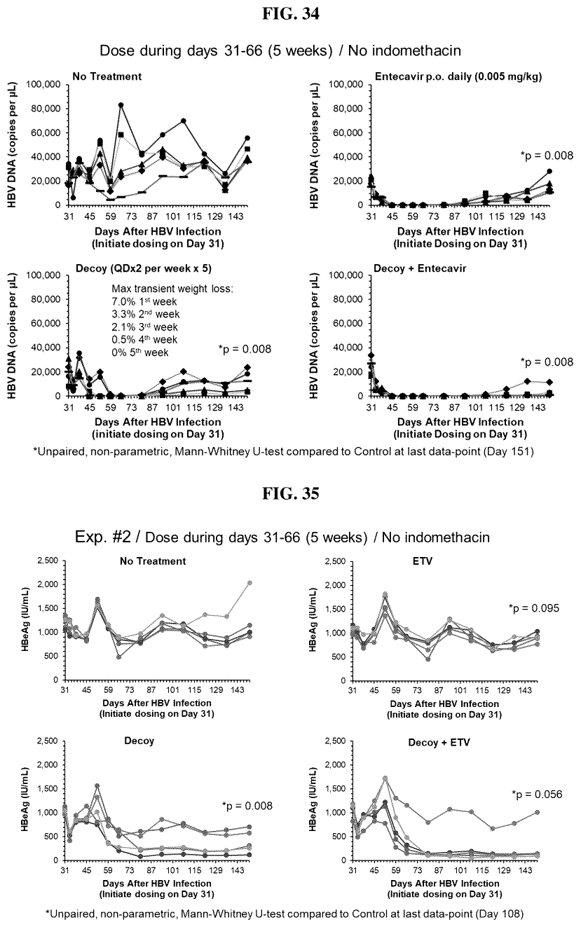

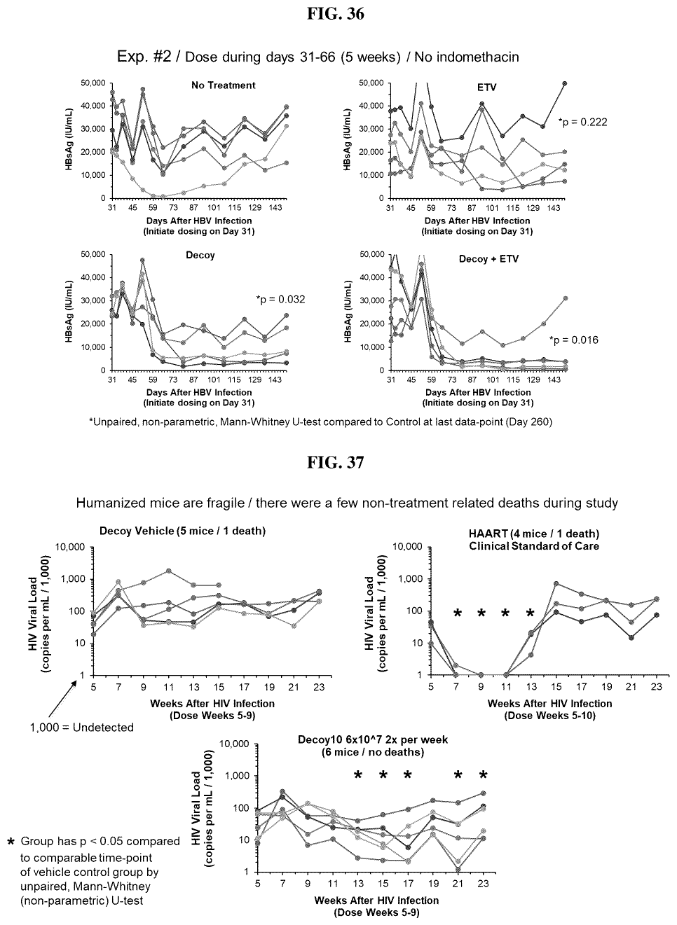

Results were similar to the first experiment, demonstrating that indomethacin is not required for Decoy activity in this model. ETV alone, Decoy alone and ETV in combination with Decoy reduced plasma HBV DNA levels during Day 31 to Day 151 without indomethacin (FIG. 34). Decoy alone and Decoy+ETV, but not ETV alone, reduced plasma HBsAg and HBeAg levels (FIG. 35 and FIG. 36). Decoy alone and Decoy in combination with ETV, but not ETV alone, reduced HBV DNA and HBeAg levels in mouse liver at termination. Decoy in combination with ETV, but not ETV alone, reduced HBsAg content in mouse liver at termination. A reduction of HBsAg content in the liver by Decoy alone was observed, but this finding was not significant. Decoy and ETV, but not ETV alone, significantly reduced liver cccDNA-like molecule expression. Decoy alone appears to have reduced the cccDNA-like molecule in 3/5 mice. Decoy induced long-lasting production of T-cell mediated anti-HB sAg activity, but did not produce anti-HBsAg B cell activity. Transient elevated plasma ALT levels were observed during treatment with Decoy, but resolved after cessation of treatment. At termination, H&E liver histopathology revealed no Decoy treatment-related changes, except minimal perivascular mononuclear cell infiltration in 2/10 mice treated with Decoy. Treatment with mIFN-alpha resulted in transient inhibition of HBV DNA in plasma, but the effect was lost shortly after cessation of treatment. No inhibition of other infection hallmarks was observed by mIFN-alpha. Combination of mIFN-alpha with Decoy did not improve upon the results obtained with Decoy alone.

### In Vivo Activity in a Mouse Model of Chronic Human HIV Infection

In this additional pharmacodynamic study, the effect of Decoy alone, and in combination with indomethacin or the human standard of care “highly active retroviral therapy” (HAART) cocktail of raltegravir, tenofovir, disproxil, and lamivudine on the HIV plasma viremia and on the counts of immune cell populations was assessed in female NOD/Shi-scid/IL-2Rγnull mice (4-6 animals per group) using a chronic human immunodeficiency virus (HIV) humanized hu-mouse model. Immune-deficient mice reconstituted with a human immune system and infected with HIV-1 were administered Decoy (6×10⁷ bacteria/animal, i.v., BIW×5 weeks), indomethacin (10 μg/mL in drinking water, 5 weeks) and/or HAART (p.o., ad libitum×5 weeks). A Decoy vehicle control was used in the study. HAART treatment significantly reduced HIV viral load in plasma within 2 weeks of starting treatment and lasting for 3 weeks after cessation of treatment. Decoy did not significantly decrease viral load during treatment, but a significant reduction was observed starting about 2-3 weeks after cessation of treatment, lasting for about 10 weeks (not all points significant) (FIG. 37). Indomethacin inhibited HIV viral load, but only during 1 week on treatment and 2 weeks off treatment, out of 18 weeks. Combinations did not appear to significantly improve single agent treatment. Human CD4+ T cell levels showed a strong decrease in all mice by Week 24. None of the agents normalized CD4+ T cell levels, although some increase in CD4+ T cells, relative to untreated mice was observed in mice treated with HAART alone or with the combination of Decoy, indomethacin and HAART.

### Summary

A comprehensive nonclinical pharmacology program has been developed to support the first in human (FIH) oncology study for Decoy. Primary pharmacodynamic (PD) studies with Decoy included in vitro assessment of induction of cytokine and chemokine secretion by murine and human peripheral blood mononuclear cells and in vivo assessment of IV anti-tumor activity against established, subcutaneous (s.c.) murine colorectal carcinoma, orthotopic murine colorectal carcinoma, metastatic murine pancreatic carcinoma, established s.c. murine hepatocellular carcinoma (HCC), established s.c. murine non-Hodgkin's lymphoma (NHL) and established s.c. human NHL models, all carried out in mice. Decoy was tested as a single agent and in combination with low-dose cyclophosphamide (LDC), an oral, low-dose non-steroidal anti-inflammatory drug (NSAID/indomethacin), murine anti-PD-1 checkpoint therapy and/or rituximab. Decoy was also tested against established, murine breast carcinoma tumors without and with expression of a foreign antigen.

Despite the ˜90% reduction in LPS-endotoxin activity, Decoy-mediated induction of cytokine and chemokine secretion by mouse and human peripheral blood mononuclear cells was largely uncompromised, with induction of multiple cytokines and chemokines responsible for activation of innate and adaptive immune cells and pathways, including those known to be required for anti-tumor and anti-viral responses. This very surprising observation may be related to a change in the mechanism and/or time-course by which immune cells process the glutaraldehyde chemically-stabilized bacteria.

Statistically significant anti-tumor activity was observed with Decoy as a single agent in a murine, s.c. colorectal carcinoma model and in a murine, metastatic pancreatic carcinoma model, as well as synergistic tumor eradication in the combination setting with LDC, indomethacin and/or anti-PD-1 therapy in a murine s.c. HCC model and a murine, s.c. NHL model. None of the agents tested consistently produced regressions or eradications as single agents. Combination based tumor eradications (tumor-free for at least 3-5 months post tumor implantation) were observed in up to 100% of animals per group and were associated with induction of 100% immunological memory, as evidenced by rejection of 100% of tumor re-challenges in the absence of additional therapy. Decoy produced single agent anti-tumor activity and combination-based 80-100% tumor eradication after only 2 to 6 weeks of once or twice per week IV treatment (depending on the model), including at well-tolerated doses with no clinical signs of toxicity, producing a therapeutic index of ≥33-fold in one combination model (HCC). Decoy also produced single agent regressions in a murine model of breast carcinoma transfected with a foreign antigen.

Anti-tumor efficacy studies with Decoy were extended to a human NHL tumor xenograft model in severe combined immunodeficient mice lacking an adaptive immune system. The Decoy+LDC combination produced high percentage regressions of established tumors, which were not durable. Addition of the standard of care targeted antibody, rituximab, produced durable, complete regressions, which is difficult to achieve in the innate only setting. Mice with regressed tumors were re-challenged with fresh tumor cells in the absence of further treatment. A subset of the new tumors were rejected, demonstrating partial innate immunological memory, which has been reported, but is also considered difficult to achieve. In conjunction with the high percentage immunological memory observed in the syngeneic setting, these results further support a dual innate+adaptive mechanism of action for killed Decoy bacteria in the combination setting.

Depletion of NK or CD4+ T cells or CD8+ T cells, prior to initiation of Decoy+LDC combination therapy in the murine NHL model resulted in an almost complete loss of tumor eradicating anti-tumor activity, further demonstrating a role for both innate and adaptive immune pathway activation. Decoy+LDC was also able to eradicate very large, up to 2,000 mm³, established s.c. tumors. In a different triple combination-based HCC tumor eradication model (Decoy+indomethacin+anti PD-1), 770-gene Nano-String gene expression analysis was carried out with s.c. tumors isolated from mice after one week of single, double, or triple combination treatment, involving a single IV dose of Decoy. Progression from single agent (no tumor regressions or eradications) to double-agent (1-2 out of 6 eradications) to triple agent (5-6 out of 6 eradications) treatment was associated with progression from low to high tumor expression of cytokine, chemokine, innate and adaptive immune pathway genes, including an increase in tumor inflammation signature score (cold to hot tumors). Tumor eradicating combination treatment was also associated with a synergistic induction of plasma cytokine and chemokine expression, in the absence of any increase in clinical signs of toxicity, relative to single agent Decoy treatment.

Primary pharmacology studies with Decoy demonstrated significant single agent and combination-based anti-tumor activity against multiple tumor types and support a mechanism of action based on priming or activation of both innate and adaptive immune cells and pathways. Significant (˜90%) reduction of the high, naturally occurring level of LPS-endotoxin activity, may enhance safety upon IV administration, while the remaining activity may be sufficient to facilitate the well-established innate and adaptive immune-stimulating properties of LPS and other TLR agonists found in Gram-negative bacteria, as well as complementing or synergizing with other immune-activating molecules in the bacteria, such as NOD and STING agonists. Secondary pharmacology studies were also carried out with Decoy, demonstrating significant single agent anti-viral activity in nonclinical models of chronic hepatitis B virus (HBV) infection and chronic human immunodeficiency virus (HIV) infection.

The safety profile of Decoy was determined after 1-hour IV infusion in single-dose, two-week repeat-dose range-finding, and four-week repeat-dose toxicology studies in New Zealand White (NZW) rabbits, which is the non-human, laboratory species considered most similar to humans with respect to sensitivity to the adverse effects of LPS. Additional safety information was obtained with Decoy in studies carried out with mice. Safety data from a twice per week four-week pivotal repeat-dose toxicology study was used to support the starting dose of Decoy in the proposed Phase 1 clinical study, involving once per week dosing.

The single-dose Decoy maximum tolerated dose (MTD) in the rabbit, with 4 dose levels tested, was determined to be 1.5×10⁹ killed bacteria [KB]/kg, due to one mortality at a dose level of 5×10⁹ KB/kg. Twice per week Decoy dosing for two weeks, with 4 dose levels tested, produced a no-observed-adverse-effect-level (NOAEL) of 6×10⁷ KB/kg/dose. In the pivotal rabbit 4-week repeat-dose toxicology study (4 dose levels), the NOAEL of Decoy was determined to be 4×10⁷ KB/kg/dose. Decoy was also found to be 97% less pyrogenic in the rabbit (rectal temperature test) than parental (untreated) bacteria and also 3-fold less toxic (acute LD₁₀₀) than parental (untreated) bacteria.

Decoy IV infusion induced minor clinical signs and symptoms which were mostly transient and reversible. These included increases in body temperature and splenic weight, changes in hematology parameters (e.g., decreased platelets, red blood cells, hematocrit, and hemoglobin) and increased white blood cells (primarily neutrophils). Changes were also noted in clinical chemistry parameters (e.g., increased alanine aminotransferase, aspartate aminotransferase, triglycerides and cholesterol, and decreased albumin and albumin/globulin ratio), as well as increased C-reactive protein and Fibrinogen. A dose-dependent increase in interleukin- (IL-) 6 plasma cytokine levels was observed shortly after administration (1.5 hours) with a return to baseline within 24 hours after the first dose and within 6 hours after the last dose. IL-6 was the only cytokine or chemokine found elevated in plasma, out of 13 tested. Only transient or no induction was observed for a variety of molecules associated with cytokine release syndromes, which represent a severe toxicity associated with many immunotherapies. Stabilization of the bacteria to prevent breakdown in the circulation, coupled with the rapid clearance of bacteria by immune cells in the liver and spleen may reduce the risk of cytokine release syndromes associated with immunotherapies that depend on continuous exposure-based dosing.

Decoy-mediated changes in platelets, white blood cells (WBCs), neutrophils, albumin, cholesterol, triglycerides, body temperature, and IL-6 were either not seen (platelets, WBC, neutrophils, and albumin) or were reduced (cholesterol, triglycerides, body temperature, and IL6) at various timepoints after subsequent doses, relative to the same timepoints after the first dose of Decoy. These results are consistent with the LPS tolerance phenomenon, which is well-documented in mice, rabbits and humans. This phenomenon should also enhance the safety of the product.

There were small incidences of macroscopic (e.g., increased splenic weight and splenomegaly [2/40 animals in the pivotal study]) and microscopic findings (e.g., minimal to mild lymphoid hyperplasia in the spleen and minimal to mild mononuclear infiltrates in the liver). Macroscopic and microscopic findings noted in the non-clinical rabbit models were considered non-adverse and are associated with the proposed mechanism of action of the product.

Nonclinical studies have demonstrated an acceptable safety profile for Decoy, both with respect to therapeutic index in pharmacology studies conducted with mice, as well as with respect to toxicology studies conducted with rabbits. Similar to well-documented findings of tolerance (decreased toxicity) with repeat IV LPS administration in mice, rabbits, and humans, Decoy, which contain LPS, exhibited the same phenomenon in mice and rabbits.

### Example 3. Clinical Testing of Decoy

This example describes a proposed clinical trial for Decoy.

The Decoy drug product consists of an attenuated and stabilized then 100% killed, non-pathogenic, Gram-negative bacterial cell suspension formulated with trehalose for cryoprotectant purposes. After diluting the cell suspension with trehalose, the final formulated drug product also contains 75% phosphate buffered saline (pH 7.5), 1.5 mM MgCl₂, and 12% trehalose. The Decoy formulated drug product is filled in 2 mL (2R) vials to a volume of 0.7 mL at ˜1.0×10⁹ killed bacteria (KB) per mL, yielding an extractable volume of 0.5 mL and stored as a frozen liquid at ≤−60° C. The target total cell count is 0.3-3.0×10⁹ cells per mL. The composition of the Decoy drug product is provided in Table 5.

TABLE 5
Composition of Decoy Drug Product

| Component | Quality | Function | Quantity (/mL) |
|---|---|---|---|
| Killed, Decoy Cells | In-house | Active | 0.3-3.0 × |
| | specification | | 10⁹ cells |
| Disodium phosphate | Ph. Eur./ | Buffer | 1.08 mg |
| dihydrate | USP | | |
| Monopotassium phosphate | Ph. Eur./ | Buffer | 0.18 mg |
| | NF | | |
| Sodium chloride | Ph. Eur./ | Buffer | 6 mg |
| | USP | | |
| Potassium chloride | Ph. Eur./ | Buffer | 0.15 mg |
| | USP | | |
| Magnesium chloride | Ph. Eur./ | Stabilizer | 0.30 mg |
| hexahydrate | USP | | |
| Trehalose dihydrate | Ph. Eur./ | Cryo- | 119.25 mg |
| | NF | protectant | |
| Water for injection | Ph. Eur./ | Solvent | qs to 1.0 mL |
| | USP | | |

Ph. Eur. = European Pharmacopoeia;
NF = National Formulary;
USP = United States Pharmacopeia.

Decoy will be diluted with sterile, 0.9% saline (normal saline) for injection and administered as a 250 mL IV infusion over approximately 1 hour. This Phase 1 study will consist of three parts. In Part 1 single ascending doses will be administered and in Part 2 (Part 2a and Part 2b) continuous weekly doses will be administered as described below.

### Part 1. Single Ascending Dose

Part 1 will be a Single Ascending Dose testing. Subjects will receive a single dose of Decoy at the assigned dose level on Week (W) 1 Day (D) 1. The dose to be administered for each single dose cohort is as described in Table 6 below.

TABLE 6
Dose Levels

| Dose Level | Dose |
|---|---|
| Cohort −1 | 3 × 10⁷ KB |
| Cohort 1 | 7 × 10⁷ KB |
| Cohort 2 | 20 × 10⁷ KB |
| Cohort 3 | 70 × 10⁷ KB |
| Cohort 4 | 200 × 10⁷ KB |
| Potential Cohort* | 400 × 10⁷ KB |

*in the event Court 4 does not produce dose-limiting toxicity (DLT).

The starting dose for Part 1 of this study (Cohort 1=7×10⁷ KB) will be ˜1/10 of the human equivalent dose (HED) determined from the no-observed-adverse-effect-level (NOAEL) observed in a twice per week 4-week rabbit toxicology study (4×10⁷ KB/kg dose). The rabbit is the closest surrogate for humans with respect to the immunological and toxicological response to IV administered purified LPS. Based on the 4-week Good Laboratory Practice (GLP) study data, including a 3.1-fold allometric scaling factor (dose decrease) to the HED plus a 10-fold dose decrease safety adjustment, the starting human dose would be 1.29×10⁶ KB per kg or approximately 16 Decoy-associated endotoxin units (EU)/kg, which is 7.74×10⁷ KB per 60 kg subject; equivalent to approximately 960 EU per 60 kg subject (not the conventional 70 kg, in order to account for a lower patient weight). The starting dose in the study will be slightly lower, at 7.0×10⁷ KB: equivalent to 868 EU per 60 kg subject or 1.8 ng/kg LPS. Since rapid clearance of Decoy by the liver and spleen is anticipated (within ˜15-30 minutes) based on testing results of systemic clearance of live and killed bacteria in mice, rabbits and humans, dose adjustment based on body weight is not considered to be necessary. The LPS in the starting dose in the study is lower than the highest dose (4 ng/kg) determined to be well-tolerated after IV administration of purified LPS to over 1,000 healthy human volunteers.

### Part 2: Continuous Weekly Administration

Part 2a will start when the single dose Recommended Phase 2 Dose (RP2D) from Part 1 is identified. The first 3 subjects enrolled into Part 2a will receive 4 doses of Decoy at the RP2D on W1D1, W2D1, W3D1, and W4D1. Safety data for each of these 3 subjects will be reviewed 4 weeks after their 4^{th} Decoy dose (W8D1). If there is no safety concern, the subject will receive continuous weekly dosing of Decoy beginning on W9D1. Once 3 subjects have completed this dosing schedule, with acceptable toxicity and approval by the Safety Review Committee (SRC), an additional 3 subjects will be enrolled to receive continuous weekly dosing of Decoy. When the 6^{th} subject completes at least 4 weekly doses, the accumulated safety data for all 6 subjects will be reviewed by the SRC to allow enrollment of subjects in Part 2b at the same or lower dose.

In Part 2b, a Dose Expansion study, subjects will receive continuous weekly Decoy at the dose and schedule determined by the SRC based on data obtained from Part 1 and Part 2a.

This study will enroll subjects with histologically confirmed diagnosis of an incurable, advanced metastatic solid tumor who have exhausted all available therapy options with proven clinical benefit for their malignancy.

The expected duration of treatment for each subject during Part 1 is 1 day (single IV infusion over approximately 1 hour), plus a 28-day dose-limiting toxicity (DLT) observation period. After the continuous weekly RP2D and dosing regimen are confirmed in Part 2a, subjects who participated in Part 1 may be allowed to receive continuous weekly dosing of Decoy at the RP2D for up to 2 years, at the discretion of the Investigator and Medical Monitor, until disease progression, intolerable toxicity, or subject withdrawal as long as they completed the Part 1 DLT observation period without a DLT, meet all eligibility criteria at the time of re-enrollment, and the Investigator feels it is in the best interest of the subject to receive continued dosing with Decoy. In addition, this protocol allows adjustment of the dose to a lower level or skipping of a dose if needed.

The first 3 subjects in Part 2a (Safety Run-In) will receive 4 weekly doses of Decoy followed by a 4week safety observation period without dosing. Based on an acceptable safety profile, these subjects will then continue with continuous weekly Decoy dosing for up to 2 years. An additional 3 subjects will receive continuous weekly Decoy dosing for up to 2 years.

Subjects in Part 2 (Part 2a and Part2b) will be administered continuous weekly Decoy for up to 2 years until disease progression, intolerable toxicity, or subject withdrawal, whichever occurs first.

Subjects who do not experience documented disease progression, unacceptable toxicity, or withdrawal of consent and who benefited from Decoy after 2 years may continue dosing of Decoy, with agreement of the Investigator and the Medical Monitor. Subjects who continue treatment may need to re-sign an Informed Consent Form (ICF) in a potential new treatment extension protocol.

### Inclusion and Exclusion Criteria

To be considered eligible to participate in this study, subjects in Part 1 (subjects in the single ascending dose portion or Part 1 selected subjects who are eligible to re-enroll into the study after the RP2D is established), Part 2a and Part 2b will follow the criteria as outlined below.

### Inclusion Criteria:

1. Must provide written informed consent by signature of an Institutional Review Board-approved informed consent form.
2. Males or females, age 18 years or older.
3. Histologically confirmed diagnosis of an advanced metastatic solid tumor.
4. Subject has received at least 1 and up to 3 lines of prior therapy in the metastatic setting, and then progressed (recurred, relapsed or are refractory) or has been intolerant. Prior therapies in the metastatic setting include chemotherapy, targeted (known molecular alterations) therapy, immunotherapy, and antibody therapy. Subjects must have exhausted all available therapy with proven clinical benefit for their malignancy. Subjects who have received>3 lines of prior therapy may be eligible, upon discussion with and approval by the Sponsor.
5. Measurable disease (at least 1 measurable lesion) per Response Evaluation Criteria in Solid Tumors (RECIST) v1.1 as defined by tumor type.
6. Eastern Cooperative Oncology Group (ECOG) performance status of 0 or 1.
7. Life expectancy of at least 3 months.
8. Female subjects must be of non-childbearing potential (surgically sterile or at least 2 years postmenopausal) or agree to use a highly effective contraception method while receiving treatment with Decoy and for 30 days after the last dose of Decoy.
   a. Females of childbearing potential must have a negative serum pregnancy test at Screening and a negative serum or urine test on W1D1 prior to Decoy dosing.
9. Male subjects must utilize reliable contraceptive precautions for the duration of Decoy treatment and 30 days after the last dose of Decoy.
10. Adequate organ function, confirmed by the following laboratory values at Screening:
   a. Neutrophils≥1200/μL
   b. Platelets≥100,000/μL (transfusion and/or growth factor support is allowable)
   c. Hemoglobin≥8.0 g/dL (transfusion and/or growth factor support is allowable)
   d. Estimated creatinine clearance≥50 mL/min as calculated using the Cockcroft-Gault formula and not dialysis-dependent.
   e. Aspartate aminotransferase (AST)≤2.5× upper limit of normal (ULN) (if liver metastasis or liver involvement—AST≤5×ULN)
   f. Alanine aminotransferase (ALT)≤2.5×ULN (if liver metastasis or liver involvement—ALT≤5×ULN)
   g. Bilirubin≤1.5×ULN (except subjects with Gilbert's syndrome)
   h. Ferritin≤4×ULN and C-Reactive Protein (CRP)≤6×ULN.
11. Left ventricular ejection fraction (LVEF)≥45% by echocardiogram (ECHO) or multi gated acquisition scan (MUGA) at Screening.
12. Must have recovered from toxicities due to prior therapies, except for peripheral neuropathy, to ≤Grade 1 according to the NCI CTCAE v5.0 criteria or to the subject's prior baseline.
13. Willing and able to comply with all scheduled visits, laboratory tests, and other study procedures.

### Exclusion Criteria:

1. Pregnant or lactating females.
2. Has an active systemic (viral, bacterial, or fungal) infection or requiring treatment. Infections should be treated, and the subject recovered prior to enrollment in the study.
3. Received radiotherapy within 28 days of the first dose of Decoy. Subjects must have recovered from all radiation-related toxicities, not require corticosteroids, and not have had radiation pneumonitis.
4. Received prior chemotherapy, immunotherapy, or major immunomodulatory therapy within 28 days or 5 half-lives from W1D1. If prior treatment with a programed cell death 1 (PD-1) or programed cell death ligand 1 (PD-L1) inhibitor (e.g., nivolumab, pembrolizumab, atezolizumab, and/or durvalumab etc.) within 2 months prior to eligibility confirmation by the Investigator.
5. Received systemic corticosteroid therapy>5 mg/day of prednisone or equivalent dose of another corticosteroid within 1 week or 5 half-lives (whichever is shorter) from W1D1 or is expected to require it during the course of the study (topical and inhaled steroids are permitted) or have Medical Monitor approval. Systemic corticosteroids are contraindicated after receiving Decoy outside of study specific needs to manage AEs.
6. Has radiographically detected primary central nervous system (CNS) or CNS metastases or symptomatic CNS involvement (including leptomeningeal carcinomatosis, cranial neuropathies or mass lesions that cause spinal cord compression).
7. Clinical evidence of significant coagulopathy during Screening (e.g., deep vein thrombosis or pulmonary embolism) or history of significant uncontrolled coagulopathy. Subjects with superficial vein thrombosis and visceral/splanchnic vein thrombosis primarily associated with the underlying disease, or with controlled coagulation profiles are eligible.
8. Has an active secondary malignancy in addition to the primary, excluding low-risk neoplasms as determined by the Investigator (e.g., non-metastatic basal cell or squamous cell skin carcinoma).
9. Has a history of or active infection with Human Immunodeficiency Virus (HIV) 1 or 2, positive read for Hepatitis B virus (HBV) antibodies or surface antigen (indicating acute HBV or chronic HBV), or positive read for Hepatitis C ([HCV] ribonucleic acid [RNA] detected by qualitative assay) at Screening. Hepatitis C RNA testing is not required in subjects with negative Hepatitis C antibody testing. HBV antibodies are not required in subjects with negative HBV surface antigen.
10. Known intolerance to non-steroidal anti-inflammatory drugs (NSAIDs).
11. Has a history of known genetic predisposition to HLH/MAS.
12. Has undergone splenectomy, has an active chronic liver disease, alcoholic liver disease, Wilson's disease, hemochromatosis, primary biliary cirrhosis, primary sclerosing cholangitis, genetic hemochromatosis, history of or planned liver transplant for end-stage liver disease of any etiology, documented history of advanced liver fibrosis or history of cirrhosis and/or hepatic decompensation including ascites, hepatic encephalopathy, or variceal bleeding.
13. Has received a live vaccine within 28 days of W1D1.
14. Has active autoimmune disease (including, but not limited to psoriasis, multiple sclerosis, lupus and rheumatoid arthritis).
15. Has a history of significant CNS disease, such as stroke (past history of transient ischemic attacks more than 6 months ago and controlled is allowed) or uncontrolled and unstable epilepsy.
16. Has severe pulmonary interstitial disease and/or oxygen saturation on room air<92%.
17. Baseline Q-T correlated (QTc) interval of >470 msec for females and >450 msec for males calculated using Fridericia's formula.
18. New York Heart Association Class III or IV cardiac disease, or myocardial ischemia or infarction within 180 days of Screening, severe unstable angina, coronary/peripheral artery bypass graft, worsening/decompensated heart failure within the past 6 months, or any other clinically significant cardiac abnormality that, in the judgement of the Investigator, would pose a health risk to the subject.
19. Major surgical procedure within 4 weeks prior to first dose of Decoy, or anticipation of need for a major surgical procedure, during the study. (Note: Placement of a central venous access catheter[s] [e.g., port, or similar] is not considered a major surgical procedure).
20. Any other acute or chronic medical or psychiatric condition that may increase the risk associated with study participation or Decoy administration or that, in the judgment of the Investigator, or Sponsor, would make the subject inappropriate for entry into the study including preexisting conditions which could increase vulnerability to expected toxicities or cytokine-induced inflammation, including abnormal blood chemistries.
21. Has received investigational therapy within 28 days or 5 half-lives (whichever is shorter) of W1D1.
22. Unwillingness or inability to comply with procedures required in this protocol.
23. Known allergy or hypersensitivity to Decoy or one of the ingredients of Decoy.

All subjects in Part 1 and Part 2 will have 1 year of long-term survival follow-up after their last dose of Decoy. These assessments may be conducted by telephone or in clinic every 3 months from the last dose of Decoy. Information on initiation of other anticancer therapy (including start date, therapy type/name, and response on treatment) may be collected.

### Example 4. Preliminary Results of Phase 1 Study of Decoy

This example describes the preliminary results of an in progress, first-in-human Phase 1 study of Decoy, an intravenous, killed, multiple immune receptor agonist bacterial product in patients with advanced solid tumors.

This is a first-in-human, open label, single dose escalation and multi-dose expansion, multicenter Phase 1 trial of Decoy in patients with advanced/metastatic solid tumors with an initial dose-limiting toxicity (DLT) period of 28 days.

Primary objectives: safety/tolerability. Secondary objectives: anti-drug immunogenicity, pharmacokinetics (PK) and preliminary anti-tumor activity. Exploratory objective: systemic immune activation via immune biomarkers. Eligible patients must have measurable tumors relapsed or refractory to standard therapies. Single-Ascending Dose (SAD) cohort evaluations precede Multiple Dose (MD) cohorts and use a standard statistical 3+3 design. The starting dose was a 1-hour i.v. infusion of 7×10⁷ killed Decoy bacteria and was based on the No Observed Adverse Effect Level in rabbits, the relevant non-clinical toxicology species for LPS.

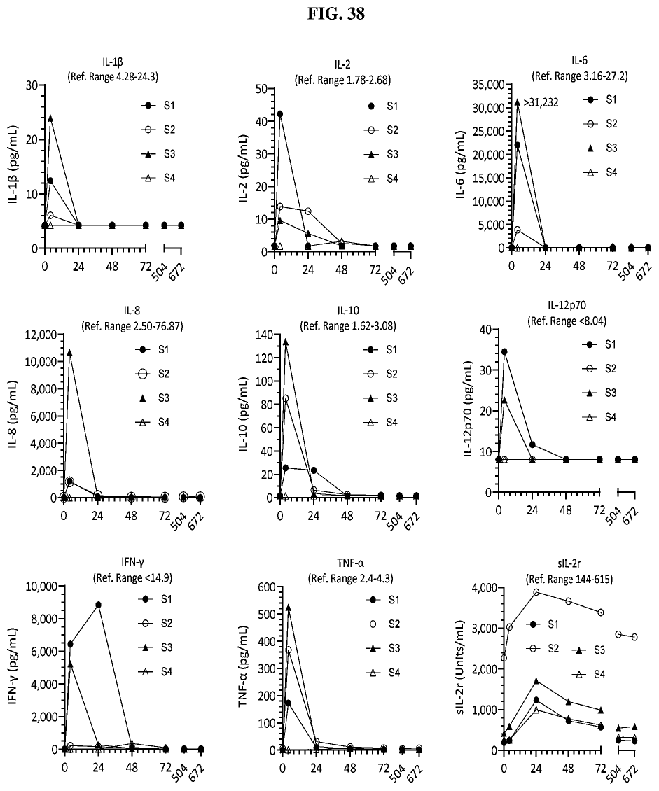

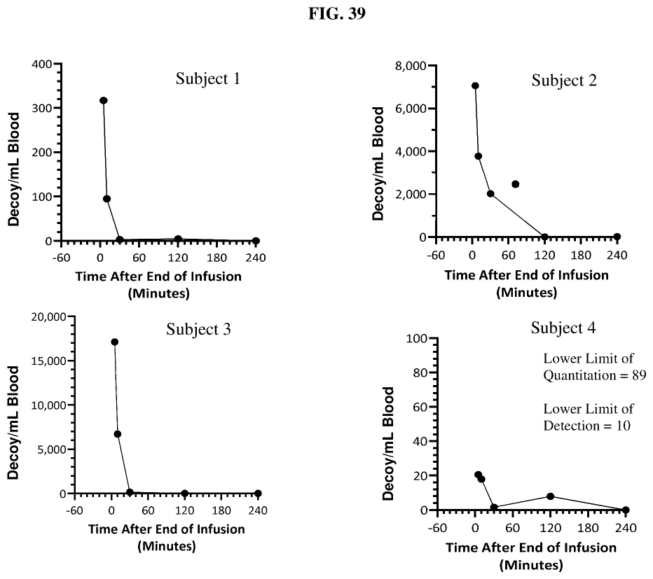

Plasma biomarkers were determined by Meso Scale Discovery Electroluminescence (FIG. 38) or Luminex Platform (FIG. 39). Decoy pharmacokinetics (PK) was determined by a digital drop (dd) PCR method.

Ethics Approval: This study was approved by the following institutions' Ethics Boards: WIRB/Copernicus covering Atlantic and Karmanos; and USC.

Four patients were enrolled for the trial, and their characteristics are shown in Table 7.

TABLE 7
Patients and Disease Characteristics

| Subject | Age, | Primary disease | | |
|---|---|---|---|---|
| # | Sex | site | Dominate sites of disease | Prior systemic therapy |
| 1 | 45, F | Tongue Adenoid | Floor of mouth and neck | Lenvatinib |
| | | Cystic CA | recurrences, measurable lung | |
| | | | metastases | |
| 2 | 71, M | Piriform sinus | Lung metastases, mediastinal | 5-FU/Carboplatin/Docetaxel; |
| | | squamous cell | lymphadenopathy | Cetuximab; Pembrolizumab; |
| | | CA | | Pembrolizumab/Trastuzumab; |
| | | | | DF1001/Nab-Paclitaxel |
| 3 | 48, M | Appendiceal | Peritoneal carcinomatosis with | FOLFOX; FOLFIRI + Bevacizumab; |
| | | adeno CA | intra-abdominal masses | Q702-ONC-P1-US001 Clinical trial; |
| | | | | VMO-01C Clinical trial |
| 4 | 47, M | Colon CA (MSS) | Lung metastases | FOLFOX; FOLFIRI + Bevacizumab |

### Results

Treatment-related adverse events are shown in Table 8. One dose limiting toxicity of Grade (G) 3 bradycardia occurred and resolved in <30 minutes following bolus normal saline, acetaminophen, meperidine, and oxygen; G3 malaise in the same patient resolved within 2 days. Two patients experienced G3 AST increase, improving to G1 within 1-2 days. Overall, G1 chills, fatigue, fever, G2 vomiting, hypotension, G1-2 ALT increase resolved within 1-2 days and G4 lymphopenia resolved within 2-3 days, all potentially expected following exposure to LPS (TLR4 agonist), an active ingredient of Decoy.

TABLE 8
Treatment-Related Adverse Events

| | INDP-D101 Cohort #/Subject # | | | |
|---|---|---|---|---|
| | 1/1 | 1/2 | 1/3 | 1/4 |
| Post-Dose Adverse Event | Grade | | | |
| Alkaline Phosphatase Increased | | G1 | | |
| ALT Increased | | G1 | G2 | |
| Anorexia | G1 | G1 | | |
| AST Increased | | G3 | G3 | |
| Bloating | | | G1 | |
| Bradycardia | | | | G3 |
| Chills | G1 | | G1 | |
| Constipation | G2 | | | |
| Creatinine Increased | | G1 | | |
| Edema | | | | |
| Fatigue | G1 | G1 | | |
| Fever | | | G1 | G1 |
| Headache | | | G1 | G2 |
| Hemoptysis | | | | G1 |
| Herpes simplex reactivation | | | G2 | |
| Hot Flashes | G1 | | | |
| Hypercalcemia | | G2 | | |
| Hypomagnesemia | G1 | | | |
| Hypophosphatemia | G2 | | | G2 |
| Hypotension | G2 | | | G2 |
| Infusion-Related Reaction (IRR) | | G2 | | G3* |
| Lymphopenia | G4 | G4 | G4 | G4 |
| Malaise | | | | G3 |
| Myalgia | | | | |
| Nausea | G1 | | | G1 |
| Paresthesia, Intermittent | G1 | | | |
| Rigors | | | | G2 |
| Tachycardia | | | G1 | |
| Vomiting | G2 | | G2 | |

*SAE due to requirement for hospitalization
Note:
Data are from an open database and are subject to change

It was observed that treatments with Decoy induced transient plasma cytokine, chemokine and biomarker expression (FIG. 38). The analysis was carried out at pre-dose, 4, 24, 48, 72 hr, 3 weeks (504 hr) and 4 weeks (672 hr) after end of infusion. Peak cytokine and chemokine induction occurred within ˜4 to 24 hours and returned to baseline by 24-48 hours. Soluble IL-2 receptor (sIL-2r), a marker of T cell activation, peaked at ˜24 hours and remained elevated up to at least 72 hours.

Table 9 provides the results of expanded single time-point plasma cytokine, chemokine and biomarker analysis. Analysis was carried out at pre-dose, 0.5, 1, 2, 4, 6, 24, 48, 72 hr, and 4 weeks after end of infusion. Most induced analytes peaked within 2-4 hr and resolved within 24-48 hours. Data in Table 9 represent the maximum fold induction or reduction.

TABLE 9
Expanded single time-point plasma cytokine, chemokine and
biomarker analysis

| | Subject | | | |
|---|---|---|---|---|
| | 1 | 2 | 3 | 4 |
| | Maximum Fold Induction ≥4-Fold | | | |
| Biomarker | or Fold Reduction ≥4-Fold | | | |
| April | 5 | | | 5 |
| BAFF | 10 | 4 | | |
| BLC (CXCL13) | 6 | 4 reduced | | 8 |
| CD30 | | | | 4 |
| CD40L (CD154) | 6 | | | |
| CRP | | | | |
| ENA-78 (CXCL5) | | | 5 | |
| Eotaxin (CCL11) | 9 | | 7 | 6 |
| Eotaxin-2 (CCL24) | | 5 reduced | | |
| Eotaxin-3 (CCL26) | | | | |
| Ferritin | 6 | | | |
| FGF-2 | | | | |
| Fractalkine (CX3CL1) | | 6 | | |
| G-CSF (CSF-3) | 18 | | 67 | 9 |
| GM-CSF | | | | 5 |
| Gro-alpha (CXCL1) | 4 | 15 | 43 | 6 |
| HGF | 8 | | 63 | 21 |
| IFN-alpha | | | | |
| IFN-beta | | | 4 | |
| IFN-gamma | 27 | (16) | (352) | 18 |
| IL-1-alpha | 7 | 17 | 5 | 5 |
| IL-1-beta | 23 | 5 | 6 | 10 |
| IL-1ra (IL-1F3) | 1,216 | 833 | N/A | N/A |
| IL-2 | (24) | (8) | (5) | |
| SIL-2r | 133 | | 5 | 120 |
| IL-3 | | | | |
| IL-4 | (12) | | 4 | 5 |
| IL-5 | | | | |
| IL-6 | 83 | 5 | 71 | 39 |
| IL-7 | | | | |
| IL-8 (CXCL8) | 31 | (33) | 31 | 24 |
| IL-9 | 6 | 13 | | |
| IL-10 | (15) | 6 | 13 | 6 |
| IL-12p40 | | | 7 | |
| IL-12p70 | 6 | | | |
| IL-13 | | | | |
| IL-15 | | | | |
| IL-16 | | 5 reduced | | 5 |
| IL-17a (CTLA-8) | | | | |
| IL-18 | 8 | 4 | | |
| IL-20 | | | | |
| IL-21 | | | 7 | |
| IL-22 | | | | |
| IL-23 | | | | |
| IL-27 | 32 | | 15 | |
| IL-31 | 14 | | | 9 |
| IL-33 | | | | |
| LIF | 6 | | 44 | |
| IP-10 (CXCL10) | 28 | | 28 | 28 |
| I-TAC (CXCL11) | 24 | 5 | 14 | 17 |
| MCP-1 (CCL2) | 40 | 19 | 22 | 51 |
| MCP-2 (CCL8) | 15 | | 10 | 25 |
| MCP-3 (CCL7) | 4 | | | |
| MCP-4 (CCL13) | 6 | 7 | 11 | |
| M-CSF | | 9 | | |
| MDC (CCL22) | | | | |
| MIF | | 8 reduced | | 18 |
| MIG (CXCL9) | 42 | | 6 | 24 |
| MIP-1a (CCL3) | 22 | | 34 | 27 |
| MIP-1b (CCL4) | 36 | 25 | 253 | 47 |
| MIP-3a (CCL20) | 25 | 25 | 92 | 78 |
| MMP-1 | | | | |
| NGF-beta | 6 | | | |
| SCF | | | 6 | 4 |
| SDF-1a (CXCL12) | 5 | | | 9 |
| TARC (CCL17) | 7 | | 25 | |
| TNF-alpha | 17 | 18 | 95 | (5) |
| TNF-beta | | | | 6 |
| TNF-RII | 5 | | | 11 |
| TRAIL (CD253) | | 6 | 8 | 7 |
| TWEAK | | | | |
| TSLP | 4 | | 25 | 4 |
| VEGF-A | 38 | | 6 | 23 |

No data entry indicates no induction ≥4-fold
N/A—not yet analyzed
Values in parenthesis from Meso Scale Discovery Electrochemiluminescence panel

Table 10 summarizes cytokines and chemokines associated with innate and adaptive anti-tumor immune responses (not exhaustive). A single dose of i.v. Decoy produced transient≥4-fold induction of the cytokines and chemokines underlined/bold in the Table 10.

TABLE 10
Cytokines and chemokines associated with innate and adaptive anti-tumor immune
responses

| Cytokines and Chemokines | Responsive Immune Cell Type: |
|---|---|
| Inducing Migration, Activation, Maturation | All Participate in |
| and/or Proliferation of Immune Cells | Anti-Tumor Immune Responses |
| GM-CSF, [symbol] , IL-4, IL-12, IL-15, IFN-α/β, [symbol] | Dendritic Cells |
| IL-2, IL-12, IL-18, [symbol] | Gamma-Delta (γσ) T-Cells |
| [symbol] , IL-8, IFN-α/β, [symbol] , [symbol] , [symbol] | M1 Macrophage |
| IL-2, IL-10, IL-12, IL-15, IL-18, IL-21, IFN-αβ, | NK Cells |
| [symbol] | |
| IL-12, IL-18, IL-21, IFN-α/β, [symbol] | NKT Cells |
| GM-CSF, IFN-α/β, IL-4, IL-8, [symbol] , [symbol] | Neutrophils |
| GM-CSF, [symbol] , IL-2, IL-5, IL-6, IL-7, IL-8, IL-9, | T-Cells (Th1, Th17 or Th2 CD4+ or CD8+) including |
| IL-10, IL-12, IL-15, IL-18, IL-21, IFN-α/β, [symbol] , | CIK, CTL, LAK |
| [symbol] , [symbol] , [symbol] | |

Pharmacokinetic analysis confirmed rapid clearance of systemically-administered Decoy. A ddPCR method with lower limit of detection/quantitation of 10/89 Decoy bacteria per mL blood was developed and used to determine Decoy levels in Subject blood pre-dose and 5, 10, 30, 120, 240 minutes, 24 hr, and 4 weeks after the end of the infusion. Decoy was cleared from blood within 30 to 120 minutes after the end of the infusion (FIG. 39). Due to peak concentration within 5 minutes of end of infusion and the steep elimination slope, differences in peak height between subjects may reflect slight differences in clearance time during infusion.

Post-dose tumor re-staging at 4 weeks demonstrated stable disease in all 4 subjects. Three of the subjects had progressive disease prior to Decoy administration.

A single i.v. dose of Decoy was cleared from blood within 30-120 minutes and produced transient induction in plasma of over 50 biomarkers, many of which have been associated with stimulation of innate and/or adaptive immune responses. Most cytokines and chemokines have been shown to play a positive role in immune responses, but can also produce toxicity if present at abnormally high levels for extended periods. Transient induction of the cytokines and chemokines is an important and novel feature of the response to Decoy bacteria, that helps to reduce the possibility of systemic toxicities that are known to result from continuous or long-term systemic exposure to these potent immune-activating molecules.

In addition, blood immune cell profiling demonstrated a rapid increase in neutrophils, and rapid decrease in essentially all other leukocytes, with recovery of all cell types within approximately 72 hours, demonstrating that Decoy induced a transient, but significant leukocyte trafficking or re-distribution event.

In summary, adverse effects were generally tolerable and resolved with or without treatment within 30 minutes to 3 days. In terms of efficacy, administration of a single dose of Decoy produced initial stable disease in all 4 subjects, including 3 progressing prior to Decoy administration.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for treating or preventing cancer in a patient in need thereof, comprising administering to the patient an effective amount of a composition comprising 1×10⁷ to 500×10⁷ intact and substantially non-viable E. coli cells which have been treated in such a way as to result in about 70% to 99% reduction of lipopolysaccharide (LPS)-associated endotoxin activity when measured by the Limulus Amebocyte Lysate (LAL) assay as compared to untreated, wild-type E. coli cells, and wherein the composition contains 124 to 62000 endotoxin units (EU) of LPS.

2. The method of claim 1, wherein the composition comprises 3×10⁷ to 100×10⁷ of the intact and substantially non-viable E. coli cells.

3. The method of claim 1, wherein the composition comprises 5×10⁷ to 50×10⁷ of the intact and substantially non-viable E. coli cells.

4. The method of claim 1, wherein the composition contains 372 EU to 8680 EU of LPS.

5. The method of claim 1, wherein the composition contains 868 EU to 2480 EU of LPS.

6. The method of claim 1, wherein the intact and substantially non-viable E. coli cells have been treated in such a way as to result in about 90% to 98% reduction of LPS-associated endotoxin.

7. The method of claim 1, wherein the administration is once every week, every two weeks, or every four weeks.

8. The method of claim 1, wherein the treatment of E. coli cells is with polymyxin.

9. The method of claim 8, wherein the treatment of E. coli cells is at a temperature from about 2° C. to about 10° C.

10. The method of claim 1, wherein the treatment of E. coli cells is with polymyxin and glutaraldehyde.

11. The method of claim 10, wherein the treatment is with polymyxin B at a dose range from about 3 mg/mL to about 1,000 mg/mL and with glutaraldehyde at a dose range from about 0.1% to about 1.0%.

12. The method of claim 1, wherein the composition further comprising a phosphate buffer, Mg²⁺, and trehalose.

13. The method of claim 12, wherein the composition comprises 0.3×10⁹/mL to 5×10⁹/mL of the intact and substantially non-viable E. coli cells, 0.5 mg/mL to 2 mg/mL of disodium phosphate dihydrate, 0.1 mg/mL to 0.4 mg/mL of monopotassium phosphate, 3 mg/mL to 12 mg/mL of sodium chloride, 0.05 mg/mL to 0.3 mg/mL of potassium chloride, 0.15 mg/mL to 0.6 mg/mL of magnesium chloride hexahydrate, and 50 mg/mL to 200 mg/mL of trehalose dihydrate, and at a pH of 7.0 to 7.7.

14. The method of claim 1, wherein the patient has a solid tumor.

15. The method of claim 14, wherein the solid tumor is a metastatic solid tumor.

16. The method of claim 1, further comprising administering to the patient a second agent selected from the group consisting of cyclophosphamide, IL-2, a non-steroidal anti-inflammatory drug (NSAID), an anti-PD-1 or anti-PD-L1 antibody, an anti-CTLA-4 antibody, and an anti-CD20 antibody.

17. A method for providing a therapeutically acceptable composition, comprising:
lyophilizing a solution comprising at least 1×10⁶ intact and substantially non-viable E. coli cells which have been treated in such a way as to result in about 70% to 99% reduction of lipopolysaccharide (LPS)-associated endotoxin activity when measured by the Limulus Amebocyte Lysate (LAL) assay as compared to untreated, wild-type E. coli cells, to prepare a lyophilized composition; and
storing the lyophilized composition (a) at a temperature of 1° C. to 10° C. for at least 2 months or (b) at a temperature of −15° C. or below for at least 2 years, thereby providing a therapeutically acceptable composition suitable for therapeutic use.

18. The method of claim 17, wherein the solution further comprises a phosphate buffer, Mg²⁺, and trehalose.

19. The method of claim 17, wherein the solution comprises 0.3×10⁹/mL to 5×10⁹/mL of the intact and substantially non-viable E. coli cells, 0.5 mg/mL to 2 mg/mL of disodium phosphate dihydrate, 0.1 mg/mL to 0.4 mg/mL of monopotassium phosphate, 3 mg/mL to 12 mg/mL of sodium chloride, 0.05 mg/mL to 0.3 mg/mL of potassium chloride, 0.15 mg/mL to 0.6 mg/mL of magnesium chloride hexahydrate, and 50 mg/mL to 200 mg/mL of trehalose dihydrate, and at a pH of 7.0 to 7.7.

20. A method for treating or preventing cancer in a patient in need thereof, comprising administering to the patient
(a) an effective amount of a composition comprising intact and substantially non-viable E. coli cells which have been treated in such a way as to result in about 70% to 99% reduction of lipopolysaccharide (LPS)-associated endotoxin activity when measured by the Limulus Amebocyte Lysate (LAL) assay as compared to untreated, wild-type E. coli cells, and
(b) an exogenous antigen associated with the cancer.
